(19) United States
(12) Patent Application Publication
(10) Pub. No.: US 2021/0137991 A1
(43) Pub. Date: May 13, 2021

(54) GENE EDITING FOR AUTOIMMUNE DISORDERS

(71) Applicant: THEMBA INC., SAN CARLOS, CA (US)

(72) Inventor: MATTHEW RABINOWITZ, HARUTSIM (IL)

(73) Assignee: THEMBA INC., SAN CARLOS, CA (US)

(21) Appl. No.: 17/054,619

(22) PCT Filed: May 14, 2019
(86) PCT No.: PCT/US2019/032214

§ 371 Date: Nov. 11, 2020

Related U.S. Application Data
(60) Provisional application No. 61/762,708, filed on Feb. 8, 2013.

(51) Int. Cl. A61K 35/28; C12N 9/20; C12N 15/113; A61P 37/00

(52) U.S. Cl. CPC A61K 35/28; A61P 37/00; C12N 15/113; C12N 9/20

(57) ABSTRACT

Provided are methods treating a subject having an autoimmune disorder comprising, for instance, decreasing, in one or more cells in the subject, the amount of one or more genetic variants associated with susceptibility to the autoimmune disorder; and/or increasing, in one or more cells in the subject, the amount of one or more genetic variants protective against the autoimmune disorder. Also provided are methods for decreasing, in the subject, the number of cells that have one or more genetic variants associated with susceptibility to the autoimmune disorder; and/or increasing, in the subject, the number of cells that have one or more genetic variants protective against the autoimmune disorder. Also provided are compositions and isolated cells for use in accordance with the methods.

### CROSS REFERENCE TO RELATED APPLICATIONS

This application claims the benefit of U.S. Provisional Application No. 62/762,708, filed on May 14, 2018, which is incorporated herein by reference in its entirety.

### SEQUENCE LISTING

The instant application contains a sequence listing which has been submitted in ASCII format via EFS-Web and is hereby incorporated by reference in its entirety. Said ASCII copy, created on May 14, 2019, is named M107385_1010WO_Sequence_Listing_ST25.txt and is 136,596 bytes in size.

### BACKGROUND

Autoimmune and immune-mediated disorders are characterized by an abnormal immune response of the body against substances and tissues normally present in the body, resulting in the killing of health body tissue. Thus, an autoimmune disorder occurs when the body's immune system attacks healthy body tissue by mistake. There are more than 80 types of autoimmune disorders, including Multiple Sclerosis (“MS”), Rheumatoid Arthritis (“RA”), Type 1 Diabetes mellitus (“T1D”), Ulcerative Colitis (“UC”), Crohn's Disease (“CD”), Eosilophinic Esophagitis, Celiac, Psoriasis and Lupus. The exact cause of autoimmune disorders is not fully known, but many are thought to have both genetic and environmental components. The existing paradigm of drug development for treatment of autoimmune disorders targets particular signaling pathways. However, the complexity of such disorders makes modeling these pathways difficult, and the resulting treatments are less effective than desired. Accordingly, there is a need in the art for a new paradigm associated with treatment of autoimmune disorders.

### SUMMARY

Provided are methods of treating a subject having an autoimmune disorder, the methods comprising decreasing, in one or more cells in the subject, the amount of one or more genetic variants associated with susceptibility to the autoimmune disorder; and/or increasing, in one or more cells in the subject, the amount of one or more genetic variants protective against the autoimmune disorder. In some aspects, the methods comprise decreasing the amount of the susceptibility genetic variant in one or more immune cells and/or one or more hematopoietic stem cells in the subject, and increasing the amount of the protective genetic variant in one or more immune cells and/or one or more hematopoietic stem cells in the subject.

Also provided are methods of treating a subject having an autoimmune disorder, the methods comprising decreasing, in the subject, the number of cells that have one or more genetic variants associated with susceptibility to the autoimmune disorder; and/or increasing, in the subject, the number of cells that have one or more genetic variants protective against the autoimmune disorder.

In some aspects the cells are immune cells and/or hematopoietic stem cells. In some aspects, the immune cells comprise one or more of leukocytes, phagocytes, macrophages, neutrophils, dendritic cells, innate lymphoid cells, eosinophils, basophils, natural killer cells, B cells, and T cells.

Some aspects comprise administering to the subject immune cells and/or hematopoietic stem cells containing the protective genetic variant; and/or immune cells and/or hematopoietic stem cells that contain the protective genetic variant and do not contain the susceptibility genetic variant. In some aspects, the proportion of protective protein variants:susceptibility protein variants in the subject (or in cells or a population of cells in the subject) is increased. That is, the amount of protective protein variants is increased relative to the amount of susceptibility protein variants.

Some aspects comprise obtaining immune cells and/or hematopoietic stem cells from a first subject, altering the obtained immune cells and/or hematopoietic stem cells to decrease the amount of the susceptibility genetic variant and/or increase the amount of the protective genetic variant, and administering the altered immune cells and/or hematopoietic stem cells to the subject in need of treatment. In some aspects, the immune cells and/or hematopoietic stem cells are obtained from the subject's blood or bone marrow. In some aspects, the first subject is the subject in need of treatment. In some aspects, the altered immune cells and/or hematopoietic stem cells are administered via venous administration or via a bone marrow transplant.

Some aspects comprise eliminating at least a portion of the hematopoietic stem cells in the subject prior to administration of the immune cells and/or hematopoietic stem cells. In some aspects, the eliminating comprises administering chemotherapy or radiation to the subject; administering anti-c-Kit monoclonal antibodies to the subject; and/or administering a CD47 blockade to the subject.

Some aspects comprise administering a genetic modifying agent to the subject, wherein the genetic modifying agent (a) decreases the amount of the susceptibility genetic variant in one or more cells in the subject, and/or (b) increases the amount of the protective genetic variant in one or more cells in the subject. In some aspects, the genetic modifying agent comprises a nuclease. In some aspects, the nuclease is (1) a class 2 clustered regularly-interspaced short palindromic repeat (CRISPR) associated nuclease, (2) a zinc finger nuclease (ZFN), (3) a Transcription Activator-Like Effector nuclease (TALEN), or (4) a meganuclease. For example, in some aspects the nuclease comprises Cas1, Cas1B, Cas2, Cas3, Cas4, Cas5, Cash, Cas7, Cas8, Cas9, Cas1O, Cpf1, Csy1, Csy2, Csy3, Csel, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, or Csf4. In some aspects, the nuclease comprises Cas9 or Cpf1.

Also provided are methods of treating a subject having an autoimmune disorder, the method comprising editing DNA in immune cells and/or hematopoietic stem cells in a subject to: (a) decrease the amount of one or more genetic variants associated with (i) resistance to a particular drug for treating the autoimmune disorder or (ii) a distribution of bacteria in the bowel of the subject associated with increased susceptibility to the autoimmune disorder; and/or (b) increase the amount of one or more genetic variants associated with (i) increased sensitivity to a particular drug for treating the autoimmune disorder or (ii) a distribution of bacterial in the bowel of a subject that is protective of the autoimmune disorder.

In some aspects, the genetic variant is a IL23R variant, a CARD9 variant, a NOD1/2 variant, a PTPN22 variant, a NADPH Oxidase Complex Gene variant, a TTC7A variant, a XIAP variant, a IL-10 variant, IL-10RA variant, a IL-10RB variant, a RPL7 variant, a CPAMD8 variant, a PRG2 variant, a PRG3 variant, a HEATR3 variant, a ATG16L1 variant, a TNFsf15 variant, a MHCII variant, a ELF1 variant, a HLA-DB1*01:03 variant, a HLA-BTNL2 variant, a ARPC2 variant, a IL12B variant, a STAT1 variant, a IRGM variant, a IRF8 variant, a TYK2 variant, a STAT3 variant, a IFNGR2 variant, a IFNGR1 variant, a RIPK2 variant, a LRRK2 variant, a C13orf31 variant, a ECM1 variant, a NKX2-3 variant, a TNF variant, a JAK1 variant, a JAK2 variant, a JAK3 variant, a TPMT variant, a NUDT15 variant, a LOC441108 variant, a PRDM1 variant, a IRGM variant, a MAGI1 variant, a CLCA2 variant, a 2q24.1 variant, or a LY75 variant. In some aspects, the autoimmune disorder comprises inflammatory bowel disease, wherein the protective genetic variant encodes a one or more of a R381Q mutation, a G149R mutation, and a V362I mutation in an IL23R protein. In some aspects, the protective genetic variant comprises a G to A mutation at rs11209026.

In some aspects, the susceptibility genetic variant and the protective genetic variant are determined based on one or more of: (a) the phenotype of one or more family members, sequencing a panel of genes in one or more family members, whole exome sequencing in one or more family members, and/or whole genome sequencing of one or more family members; (b) computer simulations of cellular signaling and/or an immune system response; (c) machine modeling of mutations that affect phenotypes, such as with linear and/or nonlinear regression models, neural networks; (d) data describing gene expression and/or gene signaling; and (e) animal models (e.g., pigs).

Also provided are isolated immune cells or hematopoietic stem cell, wherein the cellular DNA has been modified via gene editing to (a) reduce the amount of one or more genetic variants associated with susceptibility to an autoimmune disorder; and/or (b) increase the amount of one or more genetic variants protective against the autoimmune disorder.

Also provided are populations of immune cells or hematopoietic stem cells, wherein at least about 10% of the cells in the population have been modified via gene editing to (a) reduce the amount of one or more genetic variants associated with susceptibility to an autoimmune disorder; and (b) increase the amount of one or more genetic variants protective against the autoimmune disorder.

Also provided are compositions comprising (i) a nucleic acid encoding a CRISPR/Cas nuclease, (ii) a guide RNA, or a nucleic acid encoding the guide RNA, that hybridizes to a target sequence within the genomic DNA of the cell that encodes a genetic variant associated with susceptibility to an autoimmune disorder, and (iii) a DNA repair template encoding a genetic variant not associated with susceptibility to an autoimmune disorder.

In some aspects, the compositions comprise (i) a nucleic acid encoding a CRISPR/Cas nuclease, (ii) a guide RNA, or a nucleic acid encoding the guide RNA, that hybridizes to a target sequence within the genomic DNA of the cell that encodes a genetic variant not protective of an autoimmune disorder, and (iii) a DNA repair template encoding a genetic variant protective of an autoimmune disorder at the location of the target sequence.

In some aspects, the compositions comprise (a) (i) a nucleic acid encoding a CRISPR/Cas nuclease, (ii) a guide RNA, or a nucleic acid encoding the guide RNA, that hybridizes to a target sequence within the genomic DNA of the cell that encodes a genetic variant associated with susceptibility to an autoimmune disorder, and (iii) a DNA repair template encoding a genetic variant not associated with susceptibility to an autoimmune disorder; and (b) (i) a nucleic acid encoding a CRISPR/Cas nuclease, (ii) a guide RNA, or a nucleic acid encoding the guide RNA, that hybridizes to a target sequence within the genomic DNA of the cell that encodes a genetic variant not protective of an autoimmune disorder, and (iii) a DNA repair template encoding a genetic variant protective of an autoimmune disorder at the location of the target sequence.

In some aspects, the compositions comprise two or more guide RNAs, wherein the guide RNAs collectively hybridize to more than one target sequence.

In some aspects, the compositions comprise agents for targeting one or more of a IL23R variant, a CARD9 variant, a NOD1/2 variant, a PTPN22 variant, a NADPH Oxidase Complex Gene variant, a TTC7A variant, a XIAP variant, a IL-10 variant, a IL-10RA variant, a IL-10RB variant, a RPL7 variant, a CPAMD8 variant, a PRG2 variant, a PRG3 variant, a HEATR3 variant, a ATG16L1 variant, a TNFsf15 variant, a MHCII variant, a ELF1 variant, a HLA-DB1*01:03 variant, a HLA-BTNL2 variant, a ARPC2 variant, a IL12B variant, a STAT1 variant, a IRGM variant, a IRF8 variant, a TYK2 variant, a STAT3 variant, a IFNGR2 variant, a IFNGR1 variant, a RIPK2 variant, a LRRK2 variant, a C13orf31 variant, a ECM1 variant, a NKX2-3 variant, a TNF variant, a JAK1 variant, a JAK2 variant, a JAK3 variant, a TPMT variant, a NUDT15 variant, a LOC441108 variant, a PRDM1 variant, a IRGM variant, a MAGI1 variant, a CLCA2 variant, a 2q24.1 variant, or a LY75 variant, or a combination of the above.

### DETAILED DESCRIPTION

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

Unless defined otherwise, technical and scientific terms used herein have the same meaning as commonly understood by one of ordinary skill in the art to which this disclosure pertains. Definitions of common terms and techniques in molecular biology may be found in one or more of Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); Methods in Enzymology (1955) (Colowick ed.); PCR 2: A Practical Approach (1995) (M. J. MacPherson, B. D. Hames, and G. R. Taylor eds.): Antibodies, A Laboratory Manual (1988) (Harlow and Lane, eds.); Antibodies, A Laboratory Manual, 2^{nd} ed. (2013) (E. A. Greenfield ed.); Animal Cell Culture (1987) (R. I. Freshney, ed.); Benjamin Lewin, Genes IX (2008), The Encyclopedia of Molecular Biology (1994) (Kendew et al. eds.), Molecular Biology and Biotechnology: a Comprehensive Desk Reference (1995) (Meyers ed.); Singleton et al., Dictionary of Microbiology and Molecular Biology 2^{nd} ed. (1994) (Sainsbury ed.), Advanced Organic Chemistry Reactions, Mechanisms and Structure 4^{th} ed. (1992) (March ed.); and Transgenic Mouse Methods and Protocols, 2^{nd} edition (2011) (Hofker and Deursen eds.).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +1-5% or less, +/−1% or less, and +/−0.1% or less of and from the specified value, insofar as such variations are appropriate to perform in the disclosed invention. It is to be understood that the value to which the modifier “about” or “approximately” refers is itself also specifically, and preferably, disclosed.

The term “hematopoietic stem cells” or “HSCs” or “hematopoietic bone marrow stem cells” as used herein, refers to hematopoietic cells that are pluripotent stem cells or multipotent stem cells or lymphoid or myeloid (derived from bone marrow) cells that can differentiate into a hematopoietic progenitor cell (HPC) of a lymphoid, erythroid or myeloid cell lineage or proliferate as a stem cell population without initiation of further differentiation. HSCs can be obtained e.g., from bone marrow, peripheral blood, umbilical cord blood, amniotic fluid, or placental blood or embryonic stem cells. HSCs are capable of self-renewal and differentiating into or starting a pathway to becoming a mature blood cell e.g., erythrocytes (red blood cells), platelets, granulocytes (such as neutrophils, basophils and eosinophils), macrophages, B-lymphocytes, T-lymphocytes, and Natural killer cells through the process of hematopoiesis. The term “hematopoietic stem cells” encompasses “primitive hematopoietic stem cells” i.e., long-term hematopoietic stem cells (LT-HSCs), short-term hematopoietic stem cells (ST-HSCs) and multipotent progenitor cells (MPP).

The term “immune cell” as used herein generally encompasses any cell derived from a hematopoietic stem cell that plays a role in the immune response. Immune cells include, without limitation, lymphocytes, such as T cells and B cells, antigen-presenting cells (APC), dendritic cells, monocytes, macrophages, natural killer (NK) cells, mast cells, basophils, eosinophils, or neutrophils, as well as any progenitors of such cells. In certain preferred aspects, the immune cell may be a T cell. As used herein, the term “T cell” (i.e., T lymphocyte) is intended to include all cells within the T cell lineage, including thymocytes, immature T cells, mature T cells and the like. The term “T cell” may include CD4⁺ and/or CD8⁺ T cells, T helper (Tₕ) cells, e.g., Tₕ1, Tₕ2 and Tₕ17 cells, and T regulatory (T_{reg}) cells.

The term “modified” as used herein broadly denotes that an immune cell or HSC has been subjected to or manipulated by a man-made process, such as a man-made molecular or cell biology process, resulting in the modification of at least one characteristic of the cell. Such man-made process may for example be performed in vitro or in vivo.

The term “altered expression” denotes that the modification of the immune cell or HSC alters, i.e., changes or modulates, the expression of the recited gene(s) or polypeptides(s). The term “altered expression” encompasses any direction and any extent of said alteration. Hence, “altered expression” may reflect qualitative and/or quantitative change(s) of expression, and specifically encompasses both increase (e.g., activation or stimulation) or decrease (e.g., inhibition) of expression.

The terms “increased” or “increase” or “upregulated” or “upregulate” as used herein generally mean an increase by a statically significant amount. For avoidance of doubt, “increased” encompasses a statistically significant increase of at least 10% as compared to a reference level, including an increase of at least 20%, at least 30%, at least 40%, at least 50%, at least 60%, at least 70%, at least 80%, at least 90%, at least 100% or more, including, for example at least 2-fold, at least 3-fold, at least 4-fold, at least 5-fold, at least 10-fold increase or greater as compared to a reference level.

The term “reduced” or “reduce” or “decrease” or “decreased” or “downregulate” or “downregulated” as used herein generally means a decrease by a statistically significant amount relative to a reference. For avoidance of doubt, “reduced” encompasses statistically significant decrease of at least 10% as compared to a reference level, for example a decrease by at least 20%, at least 30%, at least 40%, at least 50%, at least 60%, at least 70%, at least 80%, at least 90%, or more, up to and including a 100% decrease (i.e., absent level as compared to a reference sample), or any decrease between 10-100% as compared to a reference level. The term “abolish” or “abolished” may in particular refer to a decrease by 100%, i.e., absent level as compared to a reference sample.

The terms “quantity”, “amount” and “level” are synonymous and generally well-understood in the art. The terms may particularly refer to an absolute quantification of a marker in a tested object (e.g., in or on a cell, cell population, tissue, organ, or organism, e.g., in a biological sample of a subject), or to a relative quantification of a marker in a tested object, i.e., relative to another value such as relative to a reference value, or to a range of values indicating a base-line of the marker. For example, the base-line or reference value can be obtained based on a determination of the quantity, level, or amount of a genetic variant in a subject (or in cells or populations of cells from the subject) having an autoimmune disorder or not having an autoimmune disorder, or in a subject (or in cells or populations of cells from the subject) at risk of developing an autoimmune disorder or not at risk of developing an autoimmune disorder. Such values or ranges may be obtained as conventionally known. In some cases, the quantity, amount, or level is a measured concentration. Amounts can be quantified using known techniques, such as PCR, UV absorption, calorimetry, fluorescence-based measurement, diphenylamine reaction methods, and others. See, e.g., Li, Analytical Biochemistry, 451: 18-24 (2014); Figueroa-Gonzalez, Oncol. Lett., 13(6): 3982-88 (2017); Psifidi, PLOSE ONE, 10(1): e0115960 (18 pages) (2015); Current Protocols in Protein Science (1996) (Coligan et al., eds.); and Current Protocols in Molecular Biology (2003) (Ausubel et al., eds.).

Any one or more of the several successive molecular mechanisms involved in the expression of a given gene or polypeptide may be targeted by the immune cell or HSC modification. Without limitation, these may include targeting the gene sequence (e.g., targeting the polypeptide-encoding, non-coding and/or regulatory portions of the gene sequence), the transcription of the gene into RNA, the polyadenylation and where applicable splicing and/or other post-transcriptional modifications of the RNA into mRNA, the localization of the mRNA into cell cytoplasm, where applicable other post-transcriptional modifications of the mRNA, the translation of the mRNA into a polypeptide chain, where applicable post-translational modifications of the polypeptide, and/or folding of the polypeptide chain into the mature conformation of the polypeptide. For compartmentalized polypeptides, such as secreted polypeptides and transmembrane polypeptides, this may further include targeting trafficking of the polypeptides, i.e., the cellular mechanism by which polypeptides are transported to the appropriate sub-cellular compartment or organelle, membrane, e.g. the plasma membrane, or outside the cell.

Hence, “altered expression” may particularly denote altered production of the recited gene products by the modified immune cell or HSC. As used herein, the term “gene product(s)” includes RNA transcribed from a gene (e.g., mRNA), or a polypeptide encoded by a gene or translated from RNA.

As used herein, the term “gene” refers to a nucleic acid comprising an open reading frame encoding a polypeptide, including both exon and (optionally) intron sequences. A “gene” refers to the coding sequence of a gene product, as well as non-coding regions of the gene product, including 5′UTR and 3′UTR regions, introns and the promoter of the gene product. The coding region of a gene can be a nucleotide sequence coding for an amino acid sequence or a functional RNA, such as tRNA, rRNA, catalytic RNA, siRNA, miRNA and antisense RNA. A gene can also be an mRNA or cDNA corresponding to the coding regions (e.g. exons and miRNA) optionally comprising 5′ or 3′ untranslated sequences linked thereto. A nucleic acid may encompass a single-stranded molecule or a double-stranded molecule that comprises one or more complementary strand(s) or “complement(s)” of a particular sequence comprising a molecule. As used herein, a single-stranded nucleic acid may be denoted by the prefix “ss”, and a double stranded nucleic acid by the prefix “ds”. The term “gene” may refer to the segment of DNA involved in producing a polypeptide chain, and it includes regions preceding and following the coding region as well as intervening sequences (introns and non-translated sequences, e.g., 5′- and 3′-untranslated sequences and regulatory sequences) between individual coding segments (exons). A gene can also be an amplified nucleic acid molecule produced in vitro comprising all or a part of the coding region and/or 5′- or 3′-untranslated sequences linked thereto. The term “genetic variant” is used to refer to a version of a gene, such as a wildtype version, a mutated version, or a single-nucleotide polymorphism version. Some gene variants may be associated with increased susceptibility to an autoimmune disorder. Some gene variants may be protective against an autoimmune disorder.

The term “nuclease” as used herein broadly refers to an agent, for example a protein or a small molecule, capable of cleaving a phosphodiester bond connecting nucleotide residues in a nucleic acid molecule. In some aspects, a nuclease may be a protein, e.g., an enzyme that can bind a nucleic acid molecule and cleave a phosphodiester bond connecting nucleotide residues within the nucleic acid molecule. A nuclease may be an endonuclease, cleaving a phosphodiester bonds within a polynucleotide chain, or an exonuclease, cleaving a phosphodiester bond at the end of the polynucleotide chain. Preferably, the nuclease is an endonuclease. Preferably, the nuclease is a site-specific nuclease, binding and/or cleaving a specific phosphodiester bond within a specific nucleotide sequence, which may be referred to as “recognition sequence”, “nuclease target site”, or “target site”. In some aspects, a nuclease may recognize a single stranded target site, in other aspects a nuclease may recognize a double-stranded target site, for example a double-stranded DNA target site. Some endonucleases cut a double-stranded nucleic acid target site symmetrically, i.e., cutting both strands at the same position so that the ends comprise base-paired nucleotides, also known as blunt ends. Other endonucleases cut a double-stranded nucleic acid target sites asymmetrically, i.e., cutting each strand at a different position so that the ends comprise unpaired nucleotides. Unpaired nucleotides at the end of a double-stranded DNA molecule are also referred to as “overhangs”, e.g., “5′-overhang” or “3′-overhang”, depending on whether the unpaired nucleotide(s) form(s) the 5′ or the 5′ end of the respective DNA strand.

### Target Population

Provided are methods and compositions for treating or preventing an autoimmune disorder in a subject. In some aspects, the subject has been diagnosed with an autoimmune disorder. In some aspects, the subject is at risk of developing an autoimmune disorder. Some aspects relate to methods and compositions for the treatment of any disease wherein the risk or symptoms are reduced by editing of relevant tissue involved in the disease-causing process, to add protective mutations and/or remove susceptibility mutations. Such diseases include, but are not limited to Eosilophinic Esophagitis (see Rothenberg, Gastroenterology, 148(6): 1143-57 (2015)); Celiac Disease (see Gutierrez-Achury, Nature Genetics, 47(6): 577-78 (2015)); Psoriasis (see Tsoi, Nature Communications, 8: Article 15382 (8 pages) (2017)); Type I Diabetes (see Bonifacio, Acta Diabetol. 51(3): 403-11 (2014)); Rheumatoid Arthritis (see Eyrel, Nat. Genet., 44(12): 1336-1340 (2012)); and Inflammatory Bowel Disease (see Huang, Nature, 547: 173-178 (2017)). Listed references describe a non-limiting set of genes associated with susceptibility or protection for above-mentioned phenotypes.

In some aspects, the autoimmune disorder is inflammatory bowel disease. Inflammatory bowel disease is broadly classified to include ulcerative colitis and Crohn's disease. Ulcerative colitis is a diffuse, non-specific inflammation of unknown etiology that affects the colon, and mainly invades the mucosal membrane and frequently causes erosion and ulcers. Normally, it presents with bloody diarrhea and various degrees of general symptoms. In general, it is categorized according to the spread of symptoms (pancolitis, left-sided colitis, proctitis or right-sided or segmental colitis), disease phase (such as an active phase or remission phase), severity (mild, moderate, severe) or clinical course (relapse-remission type, chronic sustained type, acute fulminant type or initial attack type). On the other hand, Crohn's disease is a disease in which granulomatous lesions accompanied by ulceration and fibrosis occur discontinuously throughout the digestive tract from the oral cavity to the anus. Although varying according to the site and range of the lesions, symptoms include fever, nutritional disorders and anemia, and systemic complications can also occur such as arthritis, iritis or liver disorders. In general, this disease is categorized according to, for example, the location of the lesions (small intestine type, small intestine-large intestine type, rectum type or gastroduodenal type) or the disease phase (such as an active phase or inactive phase). In addition, ulcerative colitis and Crohn's disease have unknown causes, there is no fundamental therapy, and it is difficult to achieve a complete cure. Consequently, there is repeated relapse and remission, thereby considerably impairing the subject's quality of life.

In some aspects, the subject is genetically susceptible to an autoimmune disorder, such as inflammatory bowel disease. For instance, in some aspects the subject has one or more alleles associated with increased risk of having or developing an autoimmune disorder (i.e., a susceptibility genetic variant). Such an increased susceptibility to an autoimmune disorder can result in the subject having an increased amount of one or more protein variants associated with the autoimmune disorder (i.e., a susceptibility protein variant) as compared to a subject that does not have or is not at increased risk of developing the autoimmune disorder.

Susceptibility genes are not limited to genes that directly impact on development of the autoimmune disorder. For instance, in some aspects the susceptibility gene is associated with the subject's response to a particular therapy. In some aspects the susceptibility gene is associated with the subject's microbiome (e.g., gut microbiome), which without being bound by theory is believed to impact the development and progression of autoimmune disorders.

Susceptibility genes can be identified by any means, including means known in the art. For instance, in some aspects the susceptibility gene is known in the art to be associated directly or indirectly with an autoimmune disorder. In some aspects, the susceptibility gene is identified based on a family tree that includes relatives displaying or being affected by a particular phenotype. In some aspects, the susceptibility gene is identified via whole exome or whole genome sequencing of one or more family members. In some aspects, the susceptibility gene is identified via computer simulations of cellular signaling and/or computer simulations of an immune system response. In some aspects, the susceptibility gene is identified by machine modeling, such as with neural networks or other linear and nonlinear regression models and/or using gene signaling networks where particular mutations are seen to disrupt gene signaling. In some aspects, the susceptibility gene is identified using animal models (e.g., pigs or mice). In some aspects, various methods of identifying susceptibility genes (e.g., one or more of identifying known susceptibility genes, identifying particular phenotypes with reference to a family tree, and whole exome or whole genome analysis) are combined to identify an individual in need to treatment. That is, in some aspects genes common to family members having an autoimmune disorder (e.g., a particular autoimmune disorder such as Ulcerative Colitis or Crohn's Disease) are determined to be susceptibility genes.

Non-limited susceptibility genes can include one or more variants of NADPH Oxidase Complex Genes (e.g. NCF2, Annexin A1), TTC7A, XIAP, NOD1/2, IL-10, IL-10RA, IL-10RB, Ashkenazi Jewish Genes (RPL7, CPAMD8, PRG2, PRG3, HEATR3), ATG16L1 (e.g. T300A is associated with changes in the microbiome), Asian Susceptibility Genes (TNFsf15, MHCII), ELF1, HLA-DB1*01:03, HLA-BTNL2, ARPC2, IL12B, STAT1, IRGM, IRF8, TYK2, STAT3, IFNGR2, IFNGR1, RIPK2, LRRK2, IL23R, C13orf31, ECM1, NKX2-3, TNF, JAK1, JAK2, JAK3, CARD9, NOD1/2, PTPN22, TPMT, NUDT15, LOC441108, PRDM1, IRGM, MAGI1, CLCA2, 2q24.1, and LY75.

In some aspects, gene-gene interactions impact a subject's susceptibility to an autoimmune disorder. Exemplary interactions include HLA-DQA1, RIT1/UBQLN4, IFNG/IL26/IL22. As a further example, pediatric CD patients have an additive gene-gene interaction involving TLR4, PSMG1, TNFRsf6B, and IRGM.

Some susceptibility genes may be identified in the context of environmental influences. For instance, a SNP of CYP2A6 may increase the incidence of CD among smokers. On the other hand, SNPs in the GSTP1 gene are associated with increased risk of UC in ex-smokers.

In some aspects, the susceptibility genes impact a subject's response to a medication. For instance, Leukopenia can be a life-threatening condition for IBD patients receiving thiopurine, which results at least in part from genetic variation in TPMT, which encodes a thiopurine S-methyltransferase. In a patient population with low frequency of TPMT mutations (Asians), a SNP in NUDT15 is associated with thiopurine-associated leukopenia. Still further, early-onset IBD patients with deficient IL-10R may benefit from allogeneic stem cell transplantation.

In some aspects, a susceptibility gene impacts management of IBD. For instance, susceptibility loci in the NOD2 gene are associated with ileal location, stenosing and penetrating behavior, and need for surgery. Likewise, in CD, fistulizing disease is associated with IL23R, LOC441108, PRDM1, NOD2, whereas the need for surgery is associated with IRGM, TNFSF 15, C13ORF31, and NOD2. Stenosing phenotype is associated with NOD2, JAK2, and ATG16L1. An MAGII variant (which encodes a protein involved in intestinal epithelial tight junction) is associated with a complicated structuring phenotype, whereas variants in CLCA2, 2q24.1, and LY75 loci are associated with ileal involvement, mild disease course, and the presence of erythema nodosum. In UC, a SNP-based risk-scoring system including 46 SNPs is able to differentiate patients with medically refractory UC from nonmedically refractory patients, thus predicting the need for surgery. Moreover, MHC is also believed to be a genetic determinant for severe UC.

### Methods of Treatment

Some aspects comprise methods and compositions that change the paradigm for treating autoimmune disorders, including inflammatory bowel diseases such as Ulcerative Colitis and/or Crohn's Disease. Some aspects involve the use of gene editing to edit the genes of the immune cells, or the tissue that generates the immune cells by hematopoiesis, which give rise to the inflammatory immune response. By changing the underlying genes to eliminate risk alleles, and/or create protective alleles, the complex gene signaling pathways do not need to be precisely understood in order to eliminate or reduce the severity of the autoimmune phenotype.

In some aspects, the subject is administered a therapy that decreases the amount of one or more protein variants associated with susceptibility to an autoimmune disorder. Such a decrease can occur in a subject or in one or more cells in the subject. In some aspects, the number of cells with one or more susceptibility genetic variants is decreased in the subject. For instance, in some aspects the subject is administered a therapy (e.g., a genetic modifying agent) that modifies a genetic variant in vivo and/or that decreases expression of a susceptibility protein variant. In some aspects, the therapy targets the susceptibility gene, e.g., with a gene editing or gene silencing technology. Some aspects involve targeting one or more of the susceptibility genes (including without limitation environmentally-mediated susceptibility genes, susceptibility genes that affect medication response, and susceptibility genes that affect management of the autoimmune disorder), and genes that interact with the susceptibility gene.

In some aspects, the methods comprise increasing the amount of one or more genetic variants protective against the autoimmune disorder. Such an increase can occur in a subject or in one or more cells in the subject. In some aspects, the number of cells with one or more protective genetic variants is increased in the subject. For instance, in some aspects the proportion of expression of protective protein variants is increased in the subject. In some aspects, the susceptibility gene is mutated via a genetic modifying agent to a wild type variant. In some aspects the susceptibility gene is mutated to a gene that is protective against the autoimmune disorder (i.e., a protective gene). In some aspects, a genetic variant that is not protective (e.g., a wildtype genetic variant that is not protective) is mutated to a gene that is protective against the autoimmune disorder.

In some aspects, the susceptibility gene and the protective gene encode variants of the same protein. In some aspects, the susceptibility gene and the protective gene encode variants of different proteins.

Some aspects comprise identifying protective variants in genes. Protective genetic variants can be identified using the same techniques discussed above with respect to identifying susceptibility genetic variants. In some aspects, the protective genetic variant comprises a G→A mutation at rs11209026 in the IL23R gene. Without being bound by theory, it is believed such a protective IL23R mutation has an Odds Ratio of roughly 1/3 for CD and UC, and will sufficiently dampen immune response to eliminate or ameliorate the symptoms of IBD. See for example Duerr, Science, 314(5804): 1461-63 (2006); see also Sivanesan, J. Biol. Chem., 291(16): 8673-8685(2016). In some aspects the protective genetic variant encodes an IL23R variant with one or more of the following mutations to wildtype-IL23R: R381Q (e.g., corresponding to rs11209026, c.1142G>A), G149R, and V3621. Alternatively or additionally, in some aspects, the protective mutation (e.g., for IBD) occurs in one or more of: CARD9, NOD2, PTPN22 and SLC11A1. Without being bound by theory, it is believed that NOD2 and PTPN22 are protective for UC but are susceptibility genes for Crohn's. Also without being bound by theory, it is believed that for PTPN22, a R620W gain of function variant is associated with CD but a R263Q loss of function variant is associated with UC. In SLC11A1, the −237C/T polymorphism at SNP rs7573065 has an Odds Ratio of roughly 2/3 for Inflammatory Bowel Disease. See for example Archer, Genes and Immunity, 16(4): 275-283 (2015). In some aspects, the protective variants encode a TYK2 (tyrosine kinase 2) variant which is protective for Rheumatoid Arthritis, including alleles P1104A (rs34536443, OR=0.66), A928V (rs35018800, OR=0.53), and I684S (rs12720356, OR=0.86, P=4.6×10⁻⁷). See for example Diogo , PLoS ONE 10(4): e0122271 (21 pages) (2015). As another example, in some aspects, the protective variants encode an IFIH1 variant which is protective for Type I Diabetes, with Odds Ratios ranging from 0.51 to 0.74, including alleles such as E627X. See for example Nejentsev, Science, 324(5925): 387-89 (2009). There are many more examples of protective variants across several diseases that could be ameliorated by genetic modifications to HSCs. See for example Harper, Nat. Rev. Genetics, 16(12): 689-701 (2015).

In some aspects, the subject is administered an agent that targets one or more susceptibility genetic variants and/or one or more protective genetic variants in immune cells and/or hematopoietic bone marrow stem cells. In some aspects, the subject is administered a genetic modifying agent to decrease the amount of susceptibility genetic variant in the subject and/or to increase the amount of protective protein variant in the subject. In some aspects, the subject is administered immune cells and/or HSCs that have been modified to decrease the amount of susceptibility genetic variant and/or increase the amount of protective genetic variant.

In some aspects, the proportion of protective genetic variant:susceptibility genetic variant is increased in the subject, or in cells or populations of cells in the subject. In some aspects, the ratio of protective protein variants:susceptibility protein variants is increased in the subject, or in cells or populations of cells in the subject.

In some aspects, HSC cells are harvested from the blood or bone marrow, and then a genetic modifying agent is used on the harvested cells ex vivo. In some aspects CD34+ cells from blood samples are isolated using immunomagnetic or immunofluorescent methods (The CD34 protein is a member of a family of single-pass transmembrane sialomucin proteins expressed on early hematopoietic and vascular-associated tissue. It is a cell surface glycoprotein and functions as a cell-cell adhesion factor and may mediate the attachment of hematopoietic stem cells to bone marrow extracellular matrix or directly to stromal cells). Although CD34+ is ubiquitously associated with HSCs, it is also found on other cell types. See for example Sidney, Stem Cells., 32(6): 1380-9 (2014). Clinically, it can be used for the selection and enrichment of hematopoietic stem cells for bone marrow transplants. CD34+ cells can be harvested from the blood using antibodies that bind to CD34 and are attached to magnetic beads or fluorescent markers to enable subsequent isolation and subsequent gene editing of these cells. These cells may also be cultured before or after gene editing. The cells may then be returned to the subject by blood transfusion or injection. In some aspects, the subject is subjected to chemotherapy or radiation to eliminate a significant portion of their bone marrow HSCs before the edited HSCs or CD34+ cells are returned to the blood, so that the bone marrow is recolonized with a significant portion of edited HSCs. In some aspects, this application uses a lower dosage of radiation or chemotherapy than the lethal dosages used for bone marrow cancer patients (e.g., to deplete the host HSCs, but not to eliminate the entire HSC population). In some aspects, prior to transfusion of the edited HSCs, antibodies that disrupt function of HSC and cause host HSC depletion are administered to the host. For example, HSCs express c-Kit (CD117), a dimeric transmembrane receptor tyrosine kinase, which is implicated in HSC function. Anti-c-Kit monoclonal antibodies can be used along with immune suppression to deplete HSCs from bone marrow niches, allowing edited HSC engraftment. In some aspects, depletion of host HSCs can be achieved without requiring immune suppression by radiation or chemotherapy, for example by administrating antibodies that disrupt HSC function and deplete HSCs along with other antibodies to make the host HSCs more vulnerable. For example, it has been shown that host HSC clearance is dependent on Fc-mediated antibody effector functions and that HSCs express CD47, a myeloid-specific immune checkpoint, which generates a “don't kill” signal and binds to SIRPα. See Chhabra, Science Translational Medicine, 8(351): 351ra105 (10 pages) (2016). Consequently, enhancing effector activity through blockade of CD47 extends anti-c-Kit conditioning to fully immunocompetent subjects. The treatment with c-Kit antibodies along with interruption of the CD47-SIRPα axis with CD47 antibodies leads to elimination of >99% of host HSCs and robust multilineage blood reconstitution after HSC transplantation. Consequently, in some aspects, anti-c-Kit monoclonal antibodies along with blockade of CD47 are used to clear subjects HSCs before transfusion of edited HSC.

In some aspects, cells that are directly involved in the body's immune response are harvested from the blood, including for example, lymphocytes (e.g., B cells, T cells and/or natural killers cells), monocytes and/or dendritic cells. Once harvested from the blood, these immune cells may be edited in vitro, optionally cultured, and then returned to the blood stream of the subject. Without being bound by theory, it is believed that many such cells have a half-life of approximately one year. Some methods for isolating these cells, according to the manufacturer instructions, include for example cell preparation tubes (CPT) containing sodium heparin manufactured by Becton Dickinson, FicollPaque Premium (density of 1.077 g/mL) manufactured by GE Healthcare, and Lymphoprep using SepMate tubes, manufactured by STEMCELL Technologies. For strengths and weaknesses of different approaches in terms of cell isolation efficiency and cell viability, see for example Grievink, Biopreservation and Biobanking, 14(5): 410-415 (2016).

Cells encoding protective genetic variants and/or not encoding susceptibility genetic variants can be administered to the subject via any known methods (e.g., via venous administration or transplant).

Some aspects involve editing of the bone marrow stem cells that manufacture the cells of the blood. In some aspects, genetic modifying agents are delivered into bone marrow. In some aspects, the delivery includes passive targeting via polymers of neutral charge and suitable size (e.g., about 150 nm); liposomes surface-modified with an anionic glutamic acid for increased distribution into bone marrow via selective uptake by macrophages and greatly decreased distribution in liver and spleen; and/or molecules such as bisphosphates which bind specifically to bone-formation surfaces. See for example Chao-Feng, Biomaterials, 155: 191-202 (2017). These delivery mechanisms, which may for example include polymer wrappers, can be used to deliver CRISPR/Cas9 or other gene editing complexes, to the bone marrow).

Some aspects involve performing stem cell transplants directly into the bone marrow with the edited stem cells, either with our without radiation to reduce the population of bone marrow stem cells with the original germline alleles. Some aspects include methods where bone marrow stem cells are placed in the patient's blood stream (e.g., allowing the cells to find their way back to the bone marrow.

In some aspects, the subject in need of treatment is administered cells that have been obtained from the subject, and then genetically modified to decrease the amount of the susceptibility genetic variant and/or increase the amount of the protective genetic variant. In some aspects, the subject in need of treatment is administered cells obtained from a separate subject (e.g., cells from the separate subject that have genetically modified or that natively encode the desired genetic variants).

### Genetic Modifying Agents

Provided are methods and compositions that can be used to make desired genetic modifications, e.g., with the use of a genetic modifying agent. The genetic modifying agent may comprise a nuclease, such as CRISPR system, a zinc finger nuclease system, a TALEN, a meganuclease, or a RNAi system.

### CRISPR Systems

In some aspects, the genetic modifying agent is a CRISPR-Cas or CRISPR system, which refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” (e.g., RNA(s) to guide Cas, such as Cas9, e.g. CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g., Shmakov, Molecular Cell, 60(3): 385-97 (2015); Zetsche, Cell, 163(3): P759-771 (2015); WO 2014/093622 (PCT/US2013/074667).

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise DNA polynucleotides or RNA polynucleotides. In some aspects, a target sequence is located in the nucleus of a cell. In some aspects, a target sequence is located in the cytoplasm of a cell.

In certain example aspects, the CRISPR effector protein may be delivered using a nucleic acid molecule encoding the CRISPR effector protein. The nucleic acid molecule encoding a CRISPR effector protein may advantageously be a codon optimized CRISPR effector protein (e.g., a sequence optimized for expression in eukaryote, e.g., humans (i.e. being optimized for expression in humans), or for another eukaryote, animal or mammal). See, e.g., WO 2014/093622 (PCT/US2013/074667).

Non-limiting examples of Cas proteins include Cas1, Cas1B, Cas2, Cas3, Cas4, Cas5, Cas6, Cas7, Cas8, Cas9 (also known as Csn1 and Csx12), Cas1O, Cpf1, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, Csf4, homologues thereof, or modified versions thereof. While Cas9 is the most widely used Cas protein, other complexes can be also be used to edit the DNA in possibly improved ways compared to Cas9. For example, Cpf1 implements a staggered cut in target DNA where there is an overhang on one strand of DNA enabling more specific DNA reassembly. Additionally, Cpf1 requires one CRISPR RNA (crRNA) for targeting while Cas9 requires both crRNA and transactivating crRNA (tracrRNA). The smaller crRNA enables multiplex genome editing since more of them can be packaged in a single vector. Additionally, whereas Cas9 cleaves the target DNA 3 nucleotide bases upstream, Cpf1 cleaves the target DNA 18-23 bases downstream from the target site, allowing the target region to remain intact and multiple rounds of cleavage at the target locus to increase the chance of a particular edit. This invention encompasses all the editing methods discussed here, as well as others, and is independent of the specific edit method used.

Some aspects involve vectors, e.g. for delivering or introducing in a cell Cas and/or RNA capable of guiding Cas to a target locus (i.e. guide RNA), but also for propagating these components. As used herein, a “vector” is a tool that allows or facilitates the transfer of an entity from one environment to another. It is a replicon, such as a plasmid, phage, or cosmid, into which another DNA segment may be inserted so as to bring about the replication of the inserted segment. Generally, a vector is capable of replication when associated with the proper control elements. In general, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g. circular); nucleic acid molecules that comprise DNA, RNA, or both; and other varieties of polynucleotides known in the art. One type of vector is a “plasmid,” which refers to a circular double stranded DNA loop into which additional DNA segments can be inserted, such as by standard molecular cloning techniques. Another type of vector is a viral vector, wherein virally-derived DNA or RNA sequences are present in the vector for packaging into a virus (e.g. retroviruses, replication defective retroviruses, adenoviruses, replication defective adenoviruses, and adeno-associated viruses (AAVs)). Viral vectors also include polynucleotides carried by a virus for transfection into a host cell. Certain vectors are capable of autonomous replication in a host cell into which they are introduced (e.g. bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors.” Common expression vectors of utility in recombinant DNA techniques are often in the form of plasmids.

Recombinant expression vectors can comprise a nucleic acid in a form suitable for expression of the nucleic acid in a host cell, which means that the recombinant expression vectors include one or more regulatory elements, which may be selected on the basis of the host cells to be used for expression, that is operatively-linked to the nucleic acid sequence to be expressed. Within a recombinant expression vector, “operably linked” is intended to mean that the nucleotide sequence of interest is linked to the regulatory element(s) in a manner that allows for expression of the nucleotide sequence (e.g. in an in vitro transcription/translation system or in a host cell when the vector is introduced into the host cell).

The vector(s) can include the regulatory element(s), e.g., promoter(s). The vector(s) can comprise Cas encoding sequences, and/or a single, but possibly also can comprise at least 3 or 8 or 16 or 32 or 48 or 50 guide RNA(s) (e.g., sgRNAs) encoding sequences, such as 1-2, 1-3, 1-4 1-5, 3-6, 3-7, 3-8, 3-9, 3-10, 3-8, 3-16, 3-30, 3-32, 3-48, 3-50 RNA(s) (e.g., sgRNAs). In a single vector there can be a promoter for each RNA (e.g., sgRNA), advantageously when there are up to about 16 RNA(s); and, when a single vector provides for more than 16 RNA(s), one or more promoter(s) can drive expression of more than one of the RNA(s), e.g., when there are 32 RNA(s), each promoter can drive expression of two RNA(s), and when there are 48 RNA(s), each promoter can drive expression of three RNA(s).

### Guide Molecules

The term “guide sequence” and “guide molecule” encompasses any polynucleotide sequence having sufficient complementarity with a target nucleic acid sequence to hybridize with the target nucleic acid sequence and direct sequence-specific binding of a nucleic acid-targeting complex to the target nucleic acid sequence. The guide sequences made using the methods disclosed herein may be a full-length guide sequence, a truncated guide sequence, a full-length sgRNA sequence, a truncated sgRNA sequence, or an E+F sgRNA sequence. In some aspects, the degree of complementarity of the guide sequence to a given target sequence, when optimally aligned using a suitable alignment algorithm, is about or more than about 50%, 60%, 75%, 80%, 85%, 90%, 95%, 97.5%, 99%, or more. In certain example aspects, the guide molecule comprises a guide sequence that may be designed to have at least one mismatch with the target sequence, such that a RNA duplex is formed between the guide sequence and the target sequence. In some aspects, the guide sequence is designed to have a stretch of two or more adjacent mismatching nucleotides, such that the degree of complementarity over the entire guide sequence is further reduced. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies), ELAND (Illumina, San Diego, Calif.), SOAP, and Maq. The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay as described herein. Similarly, cleavage of a target nucleic acid sequence (or a sequence in the vicinity thereof) may be evaluated in a test tube by providing the target nucleic acid sequence, components of a nucleic acid-targeting complex, including the guide sequence to be tested and a control guide sequence different from the test guide sequence, and comparing binding or rate of cleavage at or in the vicinity of the target sequence between the test and control guide sequence reactions. A guide sequence, and hence a nucleic acid-targeting guide RNA may be selected to target any target nucleic acid sequence.

In certain aspects, the guide sequence or spacer length of the guide molecules is from 15 to 50 nucleotides (nt). In certain aspects, the spacer length of the guide RNA is at least 15 nucleotides. In certain aspects, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In certain example aspect, the guide sequence is 15, 16, 17, 18, 19, 20, 21, 22, 23, 24, 25, 26, 27, 28, 29, 30, 31, 32, 33, 34, 35, 36, 37, 38, 39 40, 41, 42, 43, 44, 45, 46, 47 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nt.

In certain aspects, the guide molecule comprises (1) a guide sequence capable of hybridizing to a target locus and (2) a tracr mate or direct repeat sequence whereby the direct repeat sequence is located upstream (i.e., 5′) from the guide sequence. In some aspects, the seed sequence (i.e. the sequence involved with recognition and/or hybridization to the sequence at the target locus) of the guide sequence is approximately within the first 10 nucleotides of the guide sequence.

In a particular aspect the guide molecule comprises a guide sequence linked to a direct repeat sequence, wherein the direct repeat sequence comprises one or more stem loops or optimized secondary structures. In particular aspects, the direct repeat has a minimum length of 16 nts and a single stem loop. In further aspects the direct repeat has a length longer than 16 nts, preferably more than 17 nts, and has more than one stem loops or optimized secondary structures. In particular aspects the guide molecule comprises or consists of the guide sequence linked to all or part of the natural direct repeat sequence. A typical Type V or Type VI CRISPR-cas guide molecule comprises (in 3′ to 5′ direction or in 5′ to 3′ direction): a guide sequence, a first complimentary stretch (the “repeat”), a loop (which is typically 4 or 5 nucleotides long), a second complimentary stretch (the “anti-repeat” being complimentary to the repeat), and a poly A (often poly U in RNA) tail (terminator). In certain aspects, the direct repeat sequence retains its natural architecture and forms a single stem loop. In particular aspects, certain aspects of the guide architecture can be modified, for example by addition, subtraction, or substitution of features, whereas certain other aspects of guide architecture are maintained. Preferred locations for engineered guide molecule modifications, including but not limited to insertions, deletions, and substitutions include guide termini and regions of the guide molecule that are exposed when complexed with the CRISPR-Cas protein and/or target, for example the stemloop of the direct repeat sequence.

In some aspects, the CRISPR system effector protein is an RNA-targeting effector protein. In certain aspects, the CRISPR system effector protein is a Type VI CRISPR system targeting RNA (e.g., Cas13a, Cas13b, Cas13c or Cas13d). Example RNA-targeting effector proteins include Cas13b and C2c2 (also known as Cas13a). As used herein, the term “Cas13” refers to any Type VI CRISPR system targeting RNA (e.g., Cas13a, Cas13b, Cas13c or Cas13d). When the CRISPR protein is a C2c2 protein, a tracrRNA is not required. C2c2 or Cas13 have been described in Abudayyeh et al., Science, 353(6299): aaf5573-1-aaf5573-9 (2016); Shmakov, Molecular Cell, 60(3): 385-97 (2015); and Smargon, Molecular Cell, 65: 618-30 (2017).

In some aspects, one or more elements of a nucleic acid-targeting system is derived from a particular organism comprising an endogenous CRISPR RNA-targeting system. In certain example aspects, the effector protein CRISPR RNA-targeting system comprises at least one HEPN domain. In certain example aspects, the effector protein comprises a single HEPN domain. In certain other example aspects, the effector protein comprises two HEPN domains.

In certain other example aspects, the CRISPR system effector protein is a C2c2 nuclease. The activity of C2c2 may depend on the presence of two HEPN domains. These have been shown to be RNase domains, i.e. nuclease (in particular an endonuclease) cutting RNA. C2c2 HEPN may also target DNA, or potentially DNA and/or RNA. On the basis that the HEPN domains of C2c2 are at least capable of binding to and, in their wild-type form, cutting RNA, then it is preferred that the C2c2 effector protein has RNase function. See Abudayyeh, Science, 353(6299): aaf5573-1-aaf5573-9 (2016).

### Tale Systems

In some aspects, genetic modification is made by way of the transcription activator-like effector nucleases (TALENs) system. Transcription activator-like effectors (TALEs) can be engineered to bind practically any desired DNA sequence. Exemplary methods of genome editing using the TALEN system can be found for example in Cermak, Nucleic Acids Res., 39(21) :e82 (2011); Zhang, Nat. Biotechnol., 29: 149-153 (2011) and U.S. Pat. Nos. 8,450,471, 8,440,431 and 8,440,432.

Some aspects comprise using isolated, non-naturally occurring, recombinant or engineered DNA binding proteins that comprise TALE monomers as a part of their organizational structure that enable the targeting of nucleic acid sequences with improved efficiency and expanded specificity. The structure and function of TALEs is further described in, for example, Moscou, Science, 326: 1501 (2009); Boch, Science, 326: 1509-1512 (2009); and Zhang, Nat. Biotechnology, 29: 149-153 (2011).

### ZN-Finger Nucleases

In some aspects, the genetic modifying agent includes a zinc finger system. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme Fokl. (Kim, Proc. Natl. Acad. Sci. U.S.A., 91, 883-887 (1994); Kim, Proc. Natl. Acad. Sci. U.S.A., 93, 1156-1160 (1996)). Increased cleavage specificity can be attained with decreased off target activity by use of paired ZFN heterodimers, each targeting different nucleotide sequences separated by a short spacer. (Doyon, Nat. Methods, 8: 74-79 (2011)). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626.

### Meganucleases

As disclosed herein editing can be made by way of meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514; 8,133,697; 8,021,867; 8,119,361; 8,119,381; 8,124,369; and 8,129, 134.

### RNAi

In certain aspects, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred aspect, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one aspect, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21 , 22, 23, 24, 25, 26, 27, 28, 29, or 30 nucleotides in length).

As used herein “shRNA” or “small hairpin RNA” (also called stem loop) is a type of siRNA. In one aspect, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, Genes & Development, 17: 991-1008 (2003), Lim, Science, 299, 1540 (2003), Lee and Ambros, Science, 294, 862 (2001), Lau, Science, 294, 858-861 (2001), Lagos-Quintana, Current Biology, 12: 735-739 (2002), Lagos Quintana, Science, 294, 853-857 (2001), and Lagos-Quintana, RNA, 9: 175-179 (2003). Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel, Cell, 116(2): 281-297 (2004)), comprises a dsRNA molecule.

Some aspects involve methods of generating a eukaryotic cell comprising a modified or edited gene. In some aspects, the method comprises (a) introducing one or more vectors into a eukaryotic cell, wherein the one or more vectors drive expression of one or more of: Cas effector module, and a guide sequence linked to a direct repeat sequence, wherein the Cas effector module associate one or more effector domains that mediate base editing, and (b) allowing a CRISPR-Cas effector module complex to bind to a target polynucleotide to effect base editing of the target polynucleotide within said disease gene, wherein the CRISPR-Cas effector module complex comprises a Cas effector module complexed with the guide sequence that is hybridized to the target sequence within the target polynucleotide.

A further aspect relates to an isolated cell obtained or obtainable from the methods described herein comprising the composition described herein or progeny of said modified cell. In particular aspects, the cell is a eukaryotic cell, preferably a human or non-human animal cell. In some aspects the cell is an immune cell. In some aspects, the cell is a HSC.

Some aspects comprise isolated immune cells or hematopoietic stem cells, wherein the cellular DNA has been modified via gene editing to (a) reduce the amount of one or more genetic variants associated with susceptibility to an autoimmune disorder; and/or (b) increase the amount of one or more genetic variants protective against the autoimmune disorder. Some aspects comprise a population of immune cells or hematopoietic stem cells, wherein at least about 10% of the cells in the population have been modified via gene editing to (a) reduce the amount of one or more genetic variants associated with susceptibility to an autoimmune disorder; and/or (b) increase the amount of one or more genetic variants protective against the autoimmune disorder.

The invention further relates to a method for cell therapy, comprising administering to a patient in need thereof the modified cell described herein, wherein the presence of the modified cell remedies a disease in the patient.

The following examples are included as illustrative of the compositions and methods described herein. The examples are in no way intended to limit the scope of the invention. Other aspects will be apparent to those skilled in the art.

### EXAMPLES

### Example 1

### Addressing Immune Disorders with Gene Editing

This example uses CRISPR/Cas9 gene editing, which is understood in the art. CRISPR stands for Clustered Regularly Interspaced Short Palindromic Repeats. CRISPR sequences contain segments of DNA from viruses that have attacked the cell and are used by the cell to create RNA that can detect and, in combination with Cas protein, destroy DNA from similar viruses. CAS stands for CRISPR-Associated System, and genes that code for Cas proteins are found close to the CRISPR sequences in the cellular DNA. The Cas9 protein, synthetically modified and integrated with guide RNA (gRNA), is able to bind to a particular target DNA that match the gRNA and cut the DNA at both strands. This double stranded cleavage either causes the target DNA to be replaced by the replacement DNA, or initiates a process of homologous repair in the cell, where the corresponding DNA in the homologous chromosome or template DNA is copied to repair the cut DNA.

Furthermore, this example will describe experiments conducted on murine models to demonstrate that their symptoms of IBD can be ameliorated. Colonic inflammation in mice is induced chemically and/or genetically. In particular, 2,4,6-trinitro-benzene sulfonic acid (TNBS) together with ethanol is administered intrarectally; Oxazolone is administered intrarectally; or sulfated polysaccharide dextran sulfate sodium (DSS) is administered orally via drinking water. See for example Wirtz, Nature Protocols, 12(7): 1295-1309 (2017). Alternatively or additionally, mice are genetically modified induce inflammatory bowel disease. For instance, mice are genetically modified to knockout (KO) NOD1 and/or NOD2. See, e.g., Natividad, Inflammatory Bowel Diseases, 18(8): 1434-46 (2012); Zenewicz, Immunity, 29(6): 947-57 (2008). Alternatively or additionally, mice are genetically modified based on models related to IL-10 KO, IL-2 KO, TCRa KO, TGFb KO, TAK1 KO, WASP KO, or MDR1A KO, etc. See, e.g., Mizoguchi, Progress in Mol. Biol. Transl. Sci., 105: 263-320 (2012); Wirtz, Adv. Drug Deliv. Rev., 59(11): 1073-83 (2007).

More particularly, we consider two mutations. The first is a susceptibility mutation to IBD, for example a loss of function mutation on IL-10 such as for example, 113Gly→Arg, which, without being bound by theory, it is believed to cause the IL-10 to fail to induce STAT3 phosphorylation or to inhibit lipopolysaccharide (LPS)-mediated TNF-release in peripheral blood mononuclear cells. See for example Glocker, Ann. N.Y. Acad. Sci., 1246: 102-07 (2011). The second is a protective mutation for IBD on IL23R as discussed above: R381Q corresponding to rs11209026, c.1142G>A.

Four different groups of mice are used by gene editing embryos of a standard strain: Group A that have the heterozygous IL-10 susceptibility mutation and no IL23R protective mutation, Group B that do not have either the IL10 and or the IL23R mutation, Group C that have the IL10 mutation and have the IL23R protective mutation and Group D that do not have the IL10 mutation and have the IL23R protective mutation.

The mice are generated by CRISPR/Cas9 editing a common mouse strain such as C57BL/6. Alternately, mice could also be generated from strains that are less sensitive to pain, such as for example Nav1.7 KO mice. See for example Shields, J Neurosci., 38(47): 10180-10201 (2018). The experiment could also be conceptually similar using humanized mice, namely, immunodeficient mice engrafted with functional human immune systems. See for example Kenney, Am. J Transplant., 16(2): 389-97 (2016). Furthermore, the experiment would be conceptually similar using existing mouse models eliminating need for certain edits, such as for example using the C57BL/6NTac-Il10^{em8Tac} mouse model from Taconic Biosciences, which is edited from the standard C57BL/6NTac strain using CRISPR/Cas9-mediated gene editing to delete the Il10 locus (exons 1 to 5, including the proximal promoter and UTRs).

In mice for which HSCs are edited, HSCs are harvested from the bone or blood by capturing CD34+ cells (note that while CD34 is expressed on almost all human HSCs, it is expressed on roughly 20% of murine HSC. See Ogawa, Annals of the New York Academy of Sciences, 938: 139-45 (2001); however, murine expression can be stimulated and reversed in order to change the fraction of murine HSCs that express CD34 and can be harvested. See Tajima, Blood, 96(5): 1989-93 (2000)).

The harvested cells are then edited ex vivo, and returned to the blood stream of mice that have had their native HSC population substantially depleted. This can be achieved using a non-radiation, non-chemotherapy technique of administering an anti-c-Kit antibody such as ACK2 as well as an anti-CD47 antibody such as CV1mb—modified from CV1 which effectively targets human CD47 to target mouse CD47—which have been shown to substantially deplete host HSCs—achieving robust engraftment of HSCs delivered by injection from congenic donor mice, with greater than 60% donor-derived HSC chimerism in bone, and roughly 60%, 45%, 60% and 30% chimerism in blood for donor-derived myeloid cells, B cells, NK Cells and T cells respectively. See Chhabra, Science Translational Medicine, 8(351): 351ra105 (10 pages) (2016). Mice can be treated by injection with 500 mg of ACK2 on day 1, and 500 mg of CV1mb daily for 5 days starting on day 1. Transplants can commence 6 days after treatment by transferring roughly 1 million edited HSCs by injection, each day for 3 days. Roughly 24 weeks after transplant, one may count the number of B cells, T cells, NK Cells, granulocytes (neutrophils, eosinophils, basophils, and mast cells) that carry the relevant edits compared to the host's original cells, to determine whether the engraftment of the edited HSCs was successful.

Note that the experiment would be conceptually similar replacing or augmenting with other anti-CD47 antibodies such as CV1, MIAP410, or Hu5F9G4, which is currently undergoing clinical trials in humans for use in solid and hematologic cancer (ClinicalTrials NCT02216409 and NCT02367196). Furthermore, the experiment would be conceptually similar if many more engraftment procedures were undergone or the anti-c-Kit antibody and anti-CD47 antibody treatments were augmented with radiation or chemotherapy.

Gene editing can be achieved by homologous directed repair (HDR) via CRISPR/Cas9 where template DNA matching the homolog in the region of the unedited DNA but carrying the new sequence is copied to replace the DNA after the strand is cut. In particular, one quarter of the mice in Group A (Group A1) are subjected to gene editing to remove the IL10 susceptibility mutation; one quarter of the mice in Group A (Group A2) are subjected to gene editing to add the protective IL23R mutation; and one quarter of the mice in Group A (Group A3) are subjected to gene editing to add a protective IL23R mutation and to remove the IL10 mutation. One half of the mice in Group B (Group B2) are subjected to gene editing to add the IL23R mutation. One half of the mice in Group C (Group C2) are subjected to gene editing to remove the IL10 mutation. In order to have similar numbers remaining in groups A, B and C to those in groups A1, A2, A3, B2, C2, D we can begin with mouse numbers in the ratio 4:2:2:1 in groups A, B, C and C respectively.

After waiting for the edited HSCs to generate the immune cells (e.g., after about 6 months), and assuming the engraftment in the edited groups are successful, the mice are chemically induced for colitis using one of the methods described above. The severity of the phenotype in terms of weight loss, bleeding, and diarrhea is then compared across groups A, B, C, D, A1, A2, A3, B2 and C2. The table below (Table 1) shows the starting genetic status, theoretically edited genetic status, and one possible grouping of symptom levels. Without being bound by theory, if the HSC editing were perfectly efficient, we expect the symptoms to be similar across groups A3, B2, C2 and D, across groups A2 and C, and across groups A1 and B. If the protective effect of the IL23R variant more than offsets the susceptibility effect of the IL10 variant, we expect the symptoms to be ordered A to D, worst to least.

TABLE 1
Genetic status and symptom levels of various groups

| | Starting Genetic Status | | Edited Genetic Status | | Symptom |
|---|---|---|---|---|---|
| Group | IL10 | IL23R | IL10 | IL23R | Level Pos. |
| A | Yes | No | n/a | n/a | 4 |
| B | No | No | n/a | n/a | 3 |
| C | Yes | Yes | n/a | n/a | 2 |
| D | No | Yes | n/a | n/a | 1 |
| A1 | Yes | No | No | No | 3 |
| A2 | Yes | No | Yes | Yes | 2 |
| A3 | Yes | No | No | Yes | 1 |
| B2 | No | No | No | Yes | 1 |
| C2 | Yes | Yes | No | Yes | 1 |

CRISPR-Cas9 with guide RNA (gRNA) is used to confer a protective mutation to the gene IL23R. More particularly, the gRNA is designed to attach to DNA that encodes the IL23R amino acid sequence, which is set forth below:

(SEQ ID NO: 1)
MNQVTIQWDAVIALYILFSWCHGGITNINCSGHIWVEPATIFKMGMNISI
YCQAAIKNCQPRKLHFYKNGIKERFQITRINKTTARLWYKNFLEPHASMY
CTAECPKHFQETLICGKDISSGYPPDIPDEVTCVIYEYSGNMTCTWNAGK
LTYIDTKYVVHVKSLETEEEQQYLTSSYINISTDSLQGGKKYLVWVQAAN
ALGMEESKQLQIHLDDIVIPSAAVISRAETINATVPKTIIYWDSQTTIEK
VSCEMRYKATTNQTWNVKEFDTNFTYVQQSEFYLEPNIKYVFQVRCQETG
KRYWQPWSSLFFHKTPETVPQVTSKAFQHDTWNSGLTVASISTGHLTSDN
RGDIGLLLGMIVFAVMLSILSLIGIFNRSFRTGIKRRILLLIPKWLYEDI
PNMKNSNVVKMLQENSELMNNNSSEQVLYVDPMITEIKEIFIPEHKPTDY
KKENTGPLETRDYPQNSLFDNTTVVYIPDLNTGYKPQISNFLPEGSHLSN
NNEITSLTLKPPVDSLDSGNNPRLQKHPNFAFSVSSVNSLSNTIFLGELS
LILNQGECSSPDIQNSVEEETTMLLENDSPSETIPEQTLLPDEFVSCLGI
VNEELPSINTYFPQNILESHFNRISLLEK.

The corresponding DNA sequence is set forth in the sequence listing at SEQ ID NO 2.

The Crispr-CAS9 Complex is designed to cleave the DNA in the region of the R at position 381, and the template DNA is designed to achieve homologous repair so that the R at position 381 is converted to a Q, or alternatively so that that the G at nucleotide position 1142A is converted to an A. Template DNA that can achieve this purpose will match the DNA sequencer around position 1142 at the reference DNA (SEQ ID NO: 2)

Guide RNA templates are designed to target particular regions of the IL23R gene.

Exemplary murine IL23R target sequences used to construct guide RNA sequences include:

(SEQ ID NO: 3)
ATAGAACAACAGCTCGGATT
(SEQ ID NO: 4)
ACAACAACTACACGTCCATC
(SEQ ID NO: 5)
CCCTTAAGCACTGCCGACCA
(SEQ ID NO: 6)
TGTGTCATTTATGAATACTC
(SEQ ID NO: 7)
ACCATCTGAAGAGCACATAA
(SEQ ID NO: 8)
ATCTCCACTGACTCACTGCA

Exemplary guide RNA sequences that target murine IL23R comprise the following sequences:

(SEQ ID NO: 9)
AUAGAACAACAGCUCGGAUU
(SEQ ID NO: 10)
ACAACAACUACACGUCCAUC
(SEQ ID NO: 11)
CCCUUAAGCACUGCCGACCA
(SEQ ID NO: 12)
UGUGUCAUUUAUGAAUACUC
(SEQ ID NO: 13)
ACCAUCUGAAGAGCACAUAA
(SEQ ID NO: 14)
AUCUCCACUGACUCACUGCA

Exemplary human IL23R target sequences used to construct guide RNA sequences include:

(SEQ ID NO: 15)
GTGCAGTACATAGAAGCATG
(SEQ ID NO: 16)
ACAACAACTACACGTCCATC
(SEQ ID NO: 17)
CTACATAGACACAAAATACG
(SEQ ID NO: 18)
ACCAGCTGAAGAGTATGTAA
(SEQ ID NO: 19)
CCCTTTACATACTCTTCAGC
(SEQ ID NO: 20)
ACTTCATCAGGAATATCTGG

Exemplary guide RNA sequences that target human IL23R comprise the following sequences:

(SEQ ID NO: 21)
GUGCAGUACAUAGAAGCAUG
(SEQ ID NO: 22)
ACAACAACUACACGUCCAUC
(SEQ ID NO: 23)
CUACAUAGACACAAAAUACG
(SEQ ID NO: 24)
ACCAGCUGAAGAGUAUGUAA
(SEQ ID NO: 25)
CCCUUUACAUACUCUUCAGC
(SEQ ID NO: 26)
ACUUCAUCAGGAAUAUCUGG

Exemplary target sequences used to construct guide RNA sequences that target G1142A in human IL23R include:

(SEQ ID NO: 27)
TGTCAATTCTTTCTTTGATT
(SEQ ID NO: 28)
TTTAACAGATCATTCCGAAC
(SEQ ID NO: 29)
TTAACAGATCATTCCGAACT
(SEQ ID NO: 30)
CAGATCATTCCGAACTGGGT
(SEQ ID NO: 31)
CTGCAAAAACCTACCCAGTT

Exemplary guide RNA sequences that target G1142A in human IL23R comprise the following sequences:

(SEQ ID NO: 32)
UGUCAAUUCUUUCUUUGAUU
(SEQ ID NO: 33)
UUUAACAGAUCAUUCCGAAC
(SEQ ID NO: 34)
UUAACAGAUCAUUCCGAACU
(SEQ ID NO: 35)
CAGAUCAUUCCGAACUGGGU
(SEQ ID NO: 36)
CUGCAAAAACCUACCCAGUU

The wildtype human IL-10 sequence is set forth below:

(SEQ ID NO: 37)
MHSSALLCCLVLLTGVRASPGQGTQSENSCTHFPGNLPNMLRDLRDAFSR
VKTFFQMKDQLDNLLLKESLLEDFKGYLGCQALSEMIQFYLEEVMPQAEN
QDPDIKAHVNSLGENLKTLRLRLRRCHRFLPCENKSKAVEQVKNAFNKLQ
EKGIYKAMSEFDIFINYIEAYMTMKIRN

Exemplary target sequences used to construct guide RNA sequences that target in human IL10 include:

(SEQ ID NO: 38)
GAACCAAGACCCAGACATCA
(SEQ ID NO: 39)
CAAGGCGCATGTGAACTCCC
(SEQ ID NO: 40)
AAGGCGCATGTGAACTCCCT
(SEQ ID NO: 41)
AGGCGCATGTGAACTCCCTG
(SEQ ID NO: 42)
GGCGCATGTGAACTCCCTGG
(SEQ ID NO: 43)
GGGAGAACCTGAAGACCCTC
(SEQ ID NO: 44)
GCCTCAGCCTGAGGGTCTTC
(SEQ ID NO: 45)
GGGTCTTCAGGTTCTCCCCC
(SEQ ID NO: 46)
GGTCTTCAGGTTCTCCCCCA

Exemplary guide RNA sequences that target the G113R amino acid mutation in human IL-10 comprise the following sequences:

(SEQ ID NO: 47)
GAACCAAGACCCAGACAUCA
(SEQ ID NO: 48)
CAAGGCGCAUGUGAACUCCC
(SEQ ID NO: 49)
AAGGCGCAUGUGAACUCCCU
(SEQ ID NO: 50)
AGGCGCAUGUGAACUCCCUG
(SEQ ID NO: 51)
GGCGCAUGUGAACUCCCUGG
(SEQ ID NO: 52)
GGGAGAACCUGAAGACCCUC
(SEQ ID NO: 53)
GCCUCAGCCUGAGGGUCUUC
(SEQ ID NO: 54)
GGGUCUUCAGGUUCUCCCCC
(SEQ ID NO: 55)
GGUCUUCAGGUUCUCCCCCA

### Example 2

### Machine Learning Approaches to Select Gene Mutations to Edit

Machine learning techniques are used to describe the probability of developing phenotypes based on gene mutations in order to determine which genes or variants should be edited. Such techniques include for example linear regression models, logistic regression models, nonlinear regression models including gene or gene variant interactions, regression models using principal component analysis or restriction functions to increase constraints on the regression problem if it is under-determined or noisy due to too many possible genes, or too little patient data. Restriction functions on the regression parameters could include L₂ norm as used in Ridge Regression, or L₁ norm as used in the LASSO Regression. Nonlinear interactions among the genes can be captured while still maintaining a model linear in the regression parameters by logically or mathematically combining independent genetic variables to create new variables to be used in a linear model. Nonlinear interactions can also be captured using models that are nonlinear in the parameters such as Neural Networks, including Deep Learning Neural Networks, or Support Vector Machines. Several of these methods are described for example in Rabinowitz, Bioinformatics, 22: 541-549 (2006). By looking at the size of regression parameters, which is particularly simple for linear models, or by simulating different data and presenting it to nonlinear models, genes or genetic mutations having the greatest effect on the disease phenotype or risk of the disease phenotype are determined. Other techniques to identify which variants are associated with disease include tools that use gene function and gene signaling pathway data to identify genes nearby risk loci from Genome Wide Association Studies (GWAS) that are most likely causing a phenotype. See for example Pers, Nature Communications, 6, Article 5890 (9 pages) (2015).

In addition to the references mentioned already, the following references are also noted: Sands, Inflamm. Bowel Dis., 23(1): 97-106 (2017); Jinek, Science, 337: 816-821 (2012); Salerno, OncoImmunology, 5(12): e1240857-1-e1240857-14 (2016); Sivanesan, J. Biol. Chem., 291: 8673-85 (2016); Pidasheva, PLOS ONE, 6(10): e25038 (2011); SNPedia, rs11209026; US National Library of Medicine, dpSNP: rs11209026; Duerr, Science, 314(5804): 1461-63 (2006); Hazlett, Genes & Immunity, 13: 282-87 (2012); Ferguson, Gastroenterology Research and Practice, Article ID 539461 (12 pages) (2010); Mu, Biomaterials, 155: 191-202 (2018); Angermann, Nature Methods, 9: 283-89 (2012); Gong, J. R. Soc. Interface, 14: 20170320 (13 pages) (2017); Lu, Curr. Drug Targets, 15(6): 565-72 (2014); Bassaganya-Riera, Clin. Nutr., 25(3): 454-65 (2006).

1. A method of treating a subject having an autoimmune disorder, the method comprising
(a) decreasing, in one or more cells in the subject, the amount of one or more genetic variants associated with susceptibility to the autoimmune disorder (“susceptibility genetic variant(s)”); and/or
(b) increasing, in one or more cells in the subject, the amount of one or more genetic variants protective against the autoimmune disorder (“protective genetic variant(s)”).

2. The method of claim 1, comprising decreasing the amount of the susceptibility genetic variant in one or more immune cells and/or one or more hematopoietic stem cells in the subject, and
increasing the amount of the protective genetic variant in one or more immune cells and/or one or more hematopoietic stem cells in the subject.

3. (canceled)

4. The method of claim 2, wherein the cells are immune cells.

5. The method of claim 4, wherein the immune cells comprise one or more of leukocytes, phagocytes, macrophages, neutrophils, dendritic cells, innate lymphoid cells, eosinophils, basophils, natural killer cells, B cells, and T cells.

6. The method of claim 2, comprising administering to the subject
immune cells and/or hematopoietic stem cells containing the protective genetic variant; and/or
immune cells and/or hematopoietic stem cells that contain the protective genetic variant and do not contain the susceptibility genetic variant.

7. The method of claim 6, wherein a proportion of protective protein variants:susceptibility protein variants in the subject is increased.

8. The method of claim 7, further comprising obtaining immune cells and/or hematopoietic stem cells from a first subject,
altering the obtained immune cells and/or hematopoietic stem cells to decrease the amount of the susceptibility genetic variant and/or increase the amount of the protective genetic variant, and
administering the altered immune cells and/or hematopoietic stem cells to the subject in need of treatment.

9. The method of claim 8, wherein the immune cells and/or hematopoietic stem cells are obtained from the first subject's blood or bone marrow.

10. The method of claim 9, wherein the first subject is the subject in need of treatment.

11. (canceled)

12. The method of claim 8, further comprising eliminating at least a portion of the hematopoietic stem cells in the subject prior to administration of the immune cells and/or hematopoietic stem cells.

13. The method of claim 12, wherein the eliminating comprises administering chemotherapy or radiation to the subject; administering anti-c-Kit monoclonal antibodies to the subject; and/or administering a CD47 blockade to the subject.

14. The method of claim 2, comprising administering a genetic modifying agent to the subject, wherein the genetic modifying agent (a) decreases the amount of the susceptibility genetic variant in one or more cells in the subject, and/or (b) increases the amount of the protective genetic variant in one or more cells in the subject.

15. The method of claim 14, wherein the genetic modifying agent comprises a nuclease.

16. The method of claim 15, wherein the nuclease is (1) a class 2 clustered regularly-interspaced short palindromic repeat (CRISPR) associated nuclease, (2) a zinc finger nuclease (ZFN), (3) a Transcription Activator-Like Effector nuclease (TALEN), or (4) a meganuclease.

17. The method of claim 16, wherein the nuclease comprises Cas9, Cpf1, Cas1, Cas1B, Cas2, Cas3, Cas4, Cas5, Cas6, Cas7, Cas8, Cas1O, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx1O, Csx16, CsaX, Csx3, Csx1, Csx15, Csf1, Csf2, Csf3, or Csf4.

18. (canceled)

19. A method of treating a subject having an autoimmune disorder, the method comprising editing DNA in immune cells and/or hematopoietic stem cells in a subject to:
(a) decrease the amount of one or more genetic variants associated with (i) resistance to a particular drug for treating the autoimmune disorder or (ii) a distribution of bacteria in the bowel of the subject associated with increased susceptibility to the autoimmune disorder; and/or
(b) increase the amount of one or more genetic variants associated with (i) increased sensitivity to a particular drug for treating the autoimmune disorder or (ii) a distribution of bacterial in the bowel of a subject that is protective of the autoimmune disorder.

20.-24. (canceled)

25. A population of immune cells or hematopoietic stem cells, wherein at least about 10% of the cells in the population have been modified via gene editing to (a) reduce the amount of one or more genetic variants associated with susceptibility to an autoimmune disorder; and (b) increase the amount of one or more genetic variants protective against the autoimmune disorder.

26.-29. (canceled)

30. The population of claim 25, wherein the genetic variant is a IL23R variant, a CARD9 variant, a NOD1/2 variant, a PTPN22 variant, a NADPH Oxidase Complex Gene variant, a TTC7A variant, a XIAP variant, a IL-10 variant, a IL-10RA variant, a IL-10RB variant, a RPL7 variant, a CPAMD8 variant, a PRG2 variant, a PRG3 variant, a HEATR3 variant, a ATG16L1 variant, a TNFsf15 variant, a MHCII variant, a ELF1 variant, a HLA-DB1*01:03 variant, a HLA-BTNL2 variant, a ARPC2 variant, a IL12B variant, a STAT1 variant, a IRGM variant, a IRF8 variant, a TYK2 variant, a STAT3 variant, a IFNGR2 variant, a IFNGR1 variant, a RIPK2 variant, a LRRK2 variant, a C13orf31 variant, a ECM1 variant, a NKX2-3 variant, a TNF variant, a JAK1 variant, a JAK2 variant, a JAK3 variant, a TPMT variant, a NUDT15 variant, a LOC441108 variant, a PRDM1 variant, a IRGM variant, a MAGI1 variant, a CLCA2 variant, a 2q24.1 variant, or a LY75 variant, or a combination of the above.


SEQUENCE LISTING (55 sequences)

SEQ ID NO: 1 - protein, 629 aa, Homo sapiens
MNQVTIQWDAVIALYILFSWCHGGITNINCSGHIWVEPATIFKMGMNISIYCQAAIKNCQPRKLHFYKNGIKERFQITRINKTTARLWYKNFLEPHASMYCTAECPKHFQETLICGKDISSGYPPDIPDEVTCVIYEYSGNMTCTWNAGKLTYIDTKYVVHVKSLETEEEQQYLTSSYINISTDSLQGGKKYLVWVQAANALGMEESKQLQIHLDDIVIPSAAVISRAETINATVPKTIIYWDSQTTIEKVSCEMRYKATTNQTWNVKEFDTNFTYVQQSEFYLEPNIKYVFQVRCQETGKRYWQPWSSLFFHKTPETVPQVTSKAFQHDTWNSGLTVASISTGHLTSDNRGDIGLLLGMIVFAVMLSILSLIGIFNRSFRTGIKRRILLLIPKWLYEDIPNMKNSNVVKMLQENSELMNNNSSEQVLYVDPMITEIKEIFIPEHKPTDYKKENTGPLETRDYPQNSLFDNTTVVYIPDLNTGYKPQISNFLPEGSHLSNNNEITSLTLKPPVDSLDSGNNPRLQKHPNFAFSVSSVNSLSNTIFLGELSLILNQGECSSPDIQNSVEEETTMLLENDSPSETIPEQTLLPDEFVSCLGIVNEELPSINTYFPQNILESHFNRISLLEK

SEQ ID NO: 2 - DNA, 93494 bp, Homo sapiens
acaagggtggcagcctggctctgaagtggaattatgtgcttcaaacaggttgaaaggtaaataggcagcgatcatccaaatacttaaagacagctatatttccttttggaaatacatgggccaagttgttttctcttgcaagttctcatgatattccatgcagtggttactgatgaaaagggaatctataatattatatggcatcacattactttaaataataattattgcatatttccatcagttaacttctaactaggaacatttttagggcttctgttgcttcttatgaagagaagaattactattgctaacaaataatgaaagggatccctatatttctgagccaaggggaccccagtttttttaaatgttactagatattttatgcgatatgatgagccatgcattgcgttctcttgtagttttatagaaaaagctcaggttttattaaacagataaaataactgaataatgttgatcattgagtaaatgagttaattcatgtaactgattaatcaaatagtgtaactctagttgtaaactaacaacggtattattaggaatttctataacgtgttcctttcgcgttctctacagatgaaaggctttttcctcttttttagggggtgcgggggacaaggtctcactctgtcgcccaggctggagtgcaatggtgtgatcatggttcactgcaaccttcacctcccgggtagctgggatgaccgtcacatgccaccacactgggctaatttttgtatttttagtagggacagggtttcaccctgttgcccagcctgatctcgaactcctgggctcaagcaatccaccggcctctgcctcccaaagtgctgggattacaggcatgagccaccacgcctccccagatgaaacgctttttagagtctcactggtgagagcacatattgcttaatctcacttcacttttcttaattttgaatccattttctctgggagttctgaaatttttggctcttcccaagtttagctcatcaaagactagtatttctggagtgtgataagtatgtaactttggcagggagaagaattcatcaccagaagttagctcagcccaggcaccatggttcatgcctgtaatcctagcactttgggaggttgagttgggaggaccgcttgagcccaggagtttgaggttacagtgagctatgataatgccgctgcactccagcctgggctacagagcctggcccttgtctctaaaaaaatttaaaacataaaataaaaaaagaagttaactcagaggaggaggtttctgatgagaaaggctagattaggaggcactgtaatggatcaaatgccctactgagttaattcatagtgacttttgaagagtataaaattccatttatttgctttacttctccatctcttgttatatctcctaaatataccagtaacatttctttgctctgtttccttccttcctttcttccttccttctttccttcttcctccctaatcaaaggttcccatcaaatacaataattcttagggaaaaatgttatgctttttattattttactaaaatactacaatttaaacatttttcatattttttttccagagggaaacagtcttttcctgcttccagacatgaatcaggtcactattcaatgggatgcagtaatagccctttacatactcttcagctggtgtcatggaggtatggtgttttatgtatctattgctatctttcattcaacaaatggaatattgagtgattatcattttcatggttttatgttagaatctctgaagaatacaaatattattagactagaaaaaatgtgtgcataaaaattacatagaatgaaataaaaatggcccaatctccttccaaatgcaggatttctttctcaaatttcccttctagacagccctcttactcacatgtggatatttccacaaaggagcttattagtttgagttggcatgttctattgtcaaactattataactgttatataattacctgtcaaacatttggatgcaattctatttcacaaaccacttgttgatagttactttgtgccgggcacatagtaactatgtgggccattggcaagagtatgattttattcattatattcaaactttactgaaaaaagtaggctttatctaaaaaagaacaaaagattggttgagtagctagagaatggggttcagattctacagtgcctagtgttatttctaatcttacctttgaaaaatactgtagctttataatctctcggttctctactttaaagtaggagagctaattatatttgctctttagacacttagagaagcttccaggctgactctagtcttgtgtcatggtgctgaacatcaataaaagcataacagaggccagacgaggtggctaatgcctgtaatcccagcactttgggaggccgaggcgggtggatcacctgaggtcaggagttcaagatcagcctggccaacatggtgaaacgccatattagccgggcgtggtggtgctcacctgtaatcccagctactcaggaggctaaggcaggagaatcgcttgaacctggaaggcggatgttgcagtgagccgagatcgcaacactgcactgcagcctgggtgacagaatgagactgtctcaaaaaaaaaaaaaaaaaaaaaagcctaacagaaacacactgtaataggctagtttatgaaaagatacatctagtcctaaaactgttttcaatggaatcatttagttaatagaaatgataaatttaaaatgcaatagcatattcttctgaatctcttgatttaatgttttacgtgtaatttattatttttccatttatttctaggaattacaaatataaactgctctggccacatctgggtagaaccagccacaatttttaagatgggtatgaatatctctatatattgccaagcagcaattaagaactgccaaccaaggaaacttcatttttataaaaatggcatcaaagaaagatttcaaatcacaaggattaataaaacaacagctcggctttggtataaaaactttctggaaccacatgcttctatgtactgcactgctgaatgtcccaaacattttcaagagacactgatatgtggaaaagacatttcttctggatgtaagtgttggggcacatttgaaatgcaaacaaaagctagtttaacaattaactggtcattaccacactagtctaaaaatagggacaaaataatatagttcagtttttagattagtttcatacaggagctgcaaactcaaatgtctccagggccaggttggtgaactatatgaactgtagccactgcccagatccagtaatcacaggtgcccggtattcctacatcttttggcttttcaaaggaagacagaaacataaaattttaatgtgtactctctaaattttaaaatgttgccaatacattcaaaaaacaatttttcacctctaccctgaacgaacactgcatatgtgagggccagccccgttagatttaaaatgttgctcttttaatcttcttgtttatttttctaaactaatataatataattaaccttgaagcagatcccattgagaaattttgaggccaaggtttagggagagttgcaaaactagttttgtgctcccactgtactgctaggtttgtgaccttggacaagttgcttaagcctcaattcctcgcctgtaaaaggaggctaattatacaggctgtgttatgcacttaatttcaataatttatttggaagcatatgataaagtttaaagtacatcagtacccaaatccttaaaaatgcatccttttgataaaacatagggagagtctcctacagtcctctgttcctaatctcactattccaatttagaatcactgtgtttgttattgtaactattaatttttttctttttcttaaaacccaccttgaagggaataattattaatttatttaaaagcttagtcaagtcaatcttaaaaatatacgctaaattcaatgtgtgctctgagtgtcctggaggtgtttaaatcactatagtgatatgggctggctcgtataattatatacctagtttgggttctactattatgaagggtgtaaatcaataagttcagcactgttttaactcagatattcattctctatagcacttgaacccattttcagtaaaaaaataaaggaatttgtgtctttgccatgttcccgtcctccacatttttgagaatcaagcctcagttcttgcttcttcttcacctttacaacttagctggggggcagacccatatcaccactaccacctgaccccattgtggctgcttggaaagtaagaggcttccttgctctataatgaggccattgtcctacttaactctgagcagctgatcttgcaagcttttcaagagaaggaactatagaaagttgtagaggtgagttgaggaaggaaagtcataggttttggagtcaaagaataaatctaccttaaactgatgccttaaagctgctaaacttgccctaaattgtggaaattaaaccgcaggcagacatcttatctgatgtaagaaatcttcttagcttgtccacatcctcaggaaacataaccaaacctcaatgcccaaatactttactatttatcctttgggggtgggagttgggtagagagactatacaatttctttggagggcaaataagcaactttcacagttacagaaaaagcttagttgttaatttaagccagaggaaaaaaaaggaagcaaaataaacattgtctctgttaaaaaacaaaatttcagcaaatttagtttttagatctgataggtttttatgtgtgattcatgaatcggcagcatctctttctaaaaaactgatatgtgctctgagtgtcctggaggtgtttaagttactatagtgatatgggctggcttgtataattatatacttagttttggctctactattctaacagaacagttggtttttgtaaggtaggaacaagaaatagaaatttttataaaaactaccagattggttaacattgttacttcaggttattttccttgtaatggttaaagcagaggggatttccttatcatccctgctcaggttgactgggctctttctacgtggttgctgtgaattttctgttttcaggaaaaactagtctgtgggagttttacctgcttccttaaagtttcagcttggttatatgtcacttaacaggagtgacttcattttggtttggtatgtgggggcctagtgcaggagctcagtccaaaacaatggtctcctatacattttatttaacaccaccttccgtgtgttgcagtttgtataactgattactcccaaaagccctcccaagtgttggctggggggcctctgtccccaaacccaacaccaggcaatgtagaaggtaagagaatggactttgttcctcttccaggacagcctaatagaaatacattctctgccgatggctgcttagaaagaacatcactctttccacttatttaacttttgagtttgtgaattgtaactccttacataggcctctggttatgagtgtaaaaatgtaactgccaggtgagaatttactcaaacccctggattcaagctgaatgtcttcttgagctctacattcttagcaaacaaaacttcctatttaagtaggtcaaaaaagggtttttgagggttttgtggcattctagaaatcacattgctttcactggaccactttgatcacaggatatttgtcatgtgagatttattatgtcaacttatcatcactgctttgaacaaaatgattatatgtatttttttagactagagacaaattgtaaaagacaagataaatgagtcattgctttccttgaaaagctatccaaatatattttaaagtgctgtgctttaatgtataatttgtgaaatataaaccatattatcacaatttccatttttagccacaatcctagacacttcttagattcttatctaagactaaatgcacacatattatgggaaatttgttctaatcaggccacaccaatcatgggataaaatacaagatgtctttattcatttaaaaataccaaattaaaccatgcacagtcatgtttccacttaatcatttgtttaaaattttaaatatctttgactttttaaattttaacttttggactttatttacaatgatgaaagcacatttgttattcaataaaagcctctgaatatttgtttctaacattgctattaagtatgattacggaacaattaggctggcctgtgccctgcttgagtaagaaattatagagtgatctttgccagaagcattgcatctctctccattttaatccttcatgttattatcttaatcaagccaatcaattcatggaataaactttattgagtggttactatgcaccaaaaatgtgttaaatgctagaagtacaaagaagaggaaatcacggactgttttcaagaagttctcagtagaaagggatccttggctagactctgaattaacaagaaacctggcctgtgattcctgagtgtcccaataataggggaggggtgggattggcaagaaagagcaaaaacataagcccccttttgtatacctcagagtattctctagtatctagttgtatagccaggttttgttcttggatattgaataacctggcaatgaggcctaacaacaacttgatgttattatcataagcatactcacaggcccatctttacattgcttcatgttaacaaacacttatttgcatgtgtgtgtatagtcatctttttgatcttttgcaaagttaattgaccaccagacatttgtaaccccaagaattcacttcatgtagatgccgtggagtaacacagcccaaacaatctgatagccaaatcaatggcactctgtatctgaaaccagaggaaaaaataacttcagcacaaatcattgtaatcaatgagggaccacaactcaaactccaaaatgatttaaatttatgtttccacacctacttcatttaaggctcaattaaaacaaagatttattttaccctacttggagcccagtttctctccattgtaactcaatgctacaagtcgccaaaatgttgtaaagtactttttagtgtgttcttactgtttgaatataggtttttttaaaaggatattagaccttcgatatatgaaaatagttttttaattataaaatcaaattgcattgaatatagagagtttttaaaatgtagaccagcaaaaaagaaatacctatcattcccataaaaccataactttactagtaaccataatccctctttaataatttggaatatgtctttgaaatttcagaccctctctttctcagtctctcacttaaatgaaaccactaattgaaccattttgcaacctgcttttttcactttgccatatgatcatgacttcactacatatacttttaattccttccaaaggccaagtacttaaactgtcttcttcaacattgcttttggatgtgtttaggactgtattatagtttatttaaccttattttggaaaattaagtttgtttacaattttttcattgttatgaacaataagggaacatcctttaagccaaatattttatatatctatgaacatttcaataaggatgcattcttagatgtataattgctgagttgcaaaatattacacctgcttttacagattgtgatatgtaaagtgctctccataaaggctgtaccaatttgtacttccaccagaagtgtgttagaggatttccatgagtccactccaacactgtgtactatcatttttcaaaatccttgctaatttgataggcttgaatgacatctagtattgccttgatttgcagtgagaataaaatttttaatatatttgtttattgttctacaagcaatatttttggtaagcaatttgcaggtgaagtgaaagcatatatgtacttaagataaatctttggatgtaacttcagacgcataaatgtctgtattttcattctttatgagcccctatccttccatttgtcttgtcattctctgagcagtcaagtgtcccagcagtatttcacacgtagggatgaactaagagagccacacctgaaaagtgaatatttaacaaacactcaatatttctatacttaaaaaaatcttaatcttattcactattgaatgatataactgaaaattctaggtgtcattctatagttgttggggtttcataggctttagaggcccccaaaactcaatgtcatcagaactctctcatcttttccagtctgctaggtattgttttcccctgtgttggcttcattcttaaatagactaaagactgtgaaaaagcctgtttcacaatgacagcaagatggccaccagcaacttacagctcacatgctactcagagatagcagtttcaggaggaaagagtgtgcttcttttacagtggtttagaaaaagtgccttggagaattctgattggttcatcttgagtcatatgttcattcccaaaggcacactcctattagtcggcctacctcagaggcacacttctgtggagagcctcttgattgacaggtcttggggttggggtgggggtgagattggagtgaggggctggaggggatgtcagttctcaagagggaaagacgctagccaatgaaaaataaaaatgtttgccataacatggccacctggagaaagagctgggcaggaatgggggtgggatgagttgagtggagtgacaggaaaagagccttgaaatagagttgcatgaataccaaactttaaaaaaaccttgttgtgttggtggtaaaatacataacataaaatgtaccatttttaccatggttaagtgtgcagttcagtggcactaagtacacttacactgttgtacaaccatcaccaccatttatctccagaaccctcttattgcaaatggaaagtctgcagcctttaaacaaatatccacttccctctcctccaagccccagcaactgccattctactttctacaagtttgactcttccagataccttgtgtaaggggtcatgcaatgtttttcctttagggtctgtcttacttcgcttaatataatgtcttcaagtttcatccacattgtagcatgtgttaaaatttccttcctttttaaggctgagtaatatcttattgcatgtatgaacacccaacttttaaaatgagtagtggaaaatgtgcaagagaagaagatagtctgccagagtagtagaagaaaactacaaagaacccattcgcaagagccaaaggcaaagaggtttcttttcttgtctcgtttttgagacaaggtctcactgtgtcacccaggctggagtgcagtgtcttggtcatagctcactgcagcctcaaactcctaggctccaggaatccttccagcctcagcctcccaagtagctgagactacagatatgagccaccacacccagctttaaaacagagagctttcaaggagggagtgagcaaaatatcaaatgctgctcaatgacatcaaactcaaactcaatgacaaatgaccagagatggggtctgttgaagagaaagtcatcaatgaccttagcaacaatggttttggtggaattgtggaggctgaatcctgattgttttgggtttaaatgcatatgggaaatgaagaataataggctctttaaaagtgtttagctgttaagaaaaaaagaaagatacaggagtagctgggggagactgaatttaggataatatagtcaagtataagaagaagaggaatggatatttgatggtaaagtgggttgcaaggtaaggtagtagggatggaatgaggagcagggaggtgctgagtactgattggcctgacttctcaaataaaattgtcagtttctcaacggactatagagaacttcatatttcttttttttttattcttccattacaaatttattatcttttcagacaagttgagacatactaatttaaaataaattcccttttcagtgaaaagttgataagtttaatcaggtaaagctaatgattaggtaagcaatcaatgtttataattgcatcacaacttttctggccagtgactttttatttatctatttaatttttttaaattatacttgaagttttagggtacatgtgcacaacgtgcaggcttgtttcatacatatacatgtgccatgttggtgtgctgcacccattaactgatcatttaacattaggtatatctcctaatgctatccctcccccctcctcccaccacacaactggccccggtgtgtgatgttccccttcctgtgtccatgtgttctcattgttcagttcccacctatgagtgagaacatgtggtgtgatagtttgctgagaatgatggtttccagcttcatccatgtccctacaaaggacatgaactcatcattttttatggctgcatagtattccatgctatatatgtgccacatttttttaatccactctatcattgttggacatttgggttggttccaagtctttgctattgtgaatagtgccacaataaacatacgtgtgcatgtgtctttacagcagcatgatttataatcctttgggtatatactcagtagtgggatggctcagtcaaatggtatttctagttctagatccctgaggaatcgccacactgacttccacaatggttgaactagtttacagtcccaccaacagtgtaaaagtgttcctatttctccacatcctctccagcacctgttgtttcctgactttttaatgatcaccattctacctggtgtgagatggtatctcattgtggttttgatttgcatttctctgatggccagtgatgatgagcattttttcatgtgtctgttggctgcataaatgtcttcttttgagaagtgtctgttcatatctgttgcccactttttgatggggttgtttgtttttttcttgtaaatttgtttgagttctttgtagattctggatattagccctttgtcagatgagtagattgcaaaaattttctcccattctgtaggttgcctgttcactctgatggtagtttcttttgctgtgcagaagctctttagtttaattagatcccatttgtcaattttggcttttgttgccattgtttttagtgttttcatcatgaagtctttgcccatgcctatgtcctgaatggtattatctaggtattcttctagggtttttatggttttaggtcttatgtttaagtattgaatccatcttgagttaatttttgtataaggtgcaaggaagggatccagtttcagccttatgcatatggctagccagtttcccaacacaatttattaaatagggaatcctttccctattgctcgtttttgtcaggtttgtcaaagatcagatggttgtagatgtgtggtgttatttctgaggtctctgttctgttccattggtctatatatctgttttggtaccagtatcctgctgtttttgttactgtagccttgtagtatagtttgaagtcaggtagcatgatgcctccggctttgtccttttggcttaggattgacttggcgatgcaggcttgtgtttggttccatatgacctttaaactagttttttccaattctgtgaagaaagtcattggtagcttgatggggatggcattgaatctataaattaccttgggcagtatggccattttcacgatattgattcttcctccccatgagcatggaatgttcttccatttgtttgtgtcctcttttatttcattgtacagtggtttgtagttctctttgaagaggtccttcacatcccttgtaagttggattcctaggtattttattctctttgaagcaattgtgaatgggagttcacttttgatttggctctccatttgtctgttattggtgtataagaatgcttgtgatttttgcacattgattttgtatcctgagactttgctaaagttgcctatcagcttaaggagattttgggctgagacgatggggttttctagatatacaatcatgtcatctgcaaacagggacaatttgacttcctcttttcctaattgaataccctttatttccttcacctgcctgattgccctggccagaacttccaacactatgttgaataggagtggtgagagagggcatccccatcttgtgccagttttcaaagggaatgcttccagtttttgcccattcagatgatattggctgtgggtttgtcatagatagctcttattattttgagatacgtcccactaatacctaatttattgagagtttttagcatgaagcttgttgaattttgtcaaaggccttttctgcagctattgagacaatcatgtggtttttgtcattggttctgttgatatgctggattacgtttattgatttgcatatgttgaaccagccttgcctcccagggaggaagcccacttgatcatggtggataagctttttgatgtgctgttggattcggtttgccagtattttattgaggatttttgcatcgatgttcatcagagatattggtctaaaattctcttttttggttgtgtctctgccaggctttggtatcaggaagatgctggcctcataaaatgagttagggaggattccctctttttctattgattggaatagtttcagaaggaatggtaccagcttcttcttgtacctctggtagaatttgtctgtgaatccatctggtcctggactttttttggttggtaggctattaattattgcctcaatttcagagcctattattggcctattcagagattcaacttcttcctggtttagtcttgggagggtgtatgcgtccaggaatttatccatttcttctagactttctagtttatttgcatcaaggtgtttatagtattctctgatggtagtttgtatttctgtgggatcggtggtgatatcccctgtatcattttttattgcatctgtttgatgcttctctcttttcttctttattagtcttgctagtggtctatcaattttgttgattttttcaaaaaaaacagctcctggattcattgattttttgaagggttttttgtgtctctatttccttcagttctgctctgatcttagttatttcttgccttctgctagcttttgaatgtgtttgatcttgcttctccagttcttttaattgtgatgttaggttgtcaattttagatctttcctgctttctcttgtgggcatttagtgctataaatttccctctacacactgctttaaatgtgtcccagagattcgggtatgttgtgtctttgttctcgttggtttcaaagaacatctttatttctgccttcatttcgttatgtacctagtagtcattcaggagcaggttgttcagtttccatgtagttgagcagttttgagtgagtttcttaatcctgagtcctagtttgattgcactgtggtctgagagacagtttgttagaatttctgtttttttacatttgctgaggagtactttacttccgactatgtggtcaatttggaagaggtgtggtgtggtgctgagaagaatatatattctgtggatttagggtggagagttctgtagatgtctattaggtctgcttggtgcagagctgagttcaattcctggatatcattgttaactttctgtctcgttgatctgtctaatgttgacagtggggtgttaaagtctcccattattattgtgtggggagtctaagtctctttgtaggtctctaaggacttgctttatgaatctgggtgctcctgtattgggtgcatatatatttaggatagttagctcttcttgttgaattgatccctttaccattatgtaatggccttttttgtctcttttgatctttgttggtttaaagtctgttttatcagagactaggattgcaacccctgcctttttttgttttccatttgcttggtagatcttcctccatccctttattttgagcctatgtgtgtctctgcacgtgagatgggtttcctgaatacagcacgctgatgggtcttgactctttatccaatttgccagtctgtgtctttcaattggagcatttagcccatttacatttaaggttaatattgttatgtgtgaatttgatcctgtcattatgatgttagctggttattttgctcattagttgatacagtttcttcctagcctcgatggtctttacaatttggcacgtttttgcagtggctggtactggtttttcctttccatgtttattgcttccttcaggagctcttttagggcaggcctggtggtgacaaaatctctcagcatttgcttgtctgtaaagtattttatttctccttcacttatgaagcttagtttggctggatatgaaaatctgagttgaaaattcttttctttaagaatgctgaatattgacctccactctcttctggcttttagagtttctgctgagagatccgctgttagtctgatgggcttccctttgtgggtaacccgacctttctctctggctgtgcttaacattttttccttcatttcaactttggtgaatctgacaatcatgtgtcttggagttggtcttctcgaggagtatctttgtggcgttctctgtatttcctgaatctgaatgttggcctgccttgctagatttgggaagttctcctggatcctactctggggaagtatcctgcagagtgttttccagcttggttccattctccctgtcactttcaggtacaccaatcagacatagatttggtcttttcacatagtcccgtatttcttggaggctttgttcatttctttttattctttttcctctaaacttctcttctcacttcatttcattcatttgatattccatcactgataccctgtcttccagttgatcgaattggctgctgaggcttgtgcattcatcatgtagttctcgtgccatggttttcagctccatcaggtcctttaaggacttctctgcattggttattctagttagccatccgtctaatcttttttcaaggtttttaacttctttgccatgggttcaaacttcctcctttagctcggagaagtttgatcttctgaagccttcttctctcaacttgtcaaagtcattctccatccagctttgttccattgctggtgaggagctgcattcctttggaggaggagaggcgctctgatttttagaattttcagtttttctgctctgttctttccccatctttgtggttttatctatctttggtctttgatgatggtgatgtacagatggggttttggtgtggatgtcctttctgtttgttagttttccttctaacagtcaggaacctcagctgcaggtctgttggagtttgctggaggtccattccagccctgtttgcctgggtatcagcagtggaggctgcagaacagcgaatattggtgaacagcaaatgttgctgcctgattgttcctctggaagttttgtctcagaggagtacctggccatgtgacgtgtcagtctgcccctactggggggtgcctcccagttaggctactcagaggtcagggacccacttgaggaggcagtctgtccattctcagatctcaagctgtgtgctgggagaaccactactctcttcaaagctgtcagacggggacatttaagtctgcagaggtttctgctgccttttgtttagctatgccctgcccccggaggtggagtctacagaggcaggcagtcaggcctccttgagctgcagtgggctccacccagttcgagcttcccagccactttgtttacctactcaagcctcagcaatggcgggcacccctctcccagcctcgctgccaccttgcagtttgatctcagactgctgtgctagcaatgagtgaggcttgatgggcataggaccctctgagccaggcatgggatataagctcctggtgtgccatttgctgagactgtcggaaaagtgcagtattagggtgggagtgaccctattttccaggtgccatctgtcacccctttccttggctaggaaagggaattccctgaccccttgcacttcccgggtgaggcaatgcctcgccctgcttcggctcacactctgtgcactgcacccactgtcccgcacccactgtctgataaaccccagtgagatgaacccggtacctcagttggaaatgaagaaatcgttcgtcttctgcatcattcacgctgggagctgtagactggagctgttcctattcagccatcttggcttgggaccagagaacttcgtatttcttacagcacctcctaagtgttatgttttgttgcagatccgccagatattcctgatgaagtaacctgtgtcatttatgaatattcaggcaacatgacttgcacctggaatgctgggaagctcacctacatagacacaaaatacgtggtacatgtgaagaggtaggtcacttcctcacggcttcatataagcagttccaccccagttcagccagagctctgcctccagcagagatccaagaaatcagcctcaaacattaaatatataccctgatttatcccttatttcctacttatgatcagtgaaactaccaaagcccttttcaagccattaatattttcactctggcaggcaaagtgctctatgatctttctgtcctttttattcattagtaggtaactggtagcaggctcctaatggtggtacagcctagcacagttttaagccattgcttaggacgggcacagtggctcacacctgtaatcccacactttgggaggccaaggtgggtgaataacctgaggtcaggagtttgagaccagcctggccaacatggtgaaacctggtttctactgaaaatacaaaaattagccaggcattgtggcaggcatctgtaattccagctgctcaggaggctgaggcaggagaattgcttgaacccaggaggcagaggttgcagtgagccgagactgtgccactgcactctagcctgggcgacagaacaagactccatctcaaaaaataaaataaaataataaattattgcttaaatccctgctctattcttcttctagggtttcaacaacttaatttccccgaattgctattatttttaatctttaaatgggcctaaaaataaaacataacacacaggtttgttctaattaattatgtggaatttggcttttaaagtgtatataattatgccatattaccactaaattcatggaagctattgttaataatgaccctcagatcttaaatttagtgtctacatagctgatcaactttcctgtttgccttatactaagggggttttcaggacaaaggattttcagttttaaaacctggaaagttgtgggcaaactaggacaagttgcttaccttatccgtgtttcttgttttgtttagtttattaaaaaataaaattccaggccaggcatagtggctcatgcctgtaatcccagcactttgggagactgaggctggtggatcacccgaggtcaggagttcaagatcagcctggccaacatggtgaaaccccatctctactaaaaatacaaaaattagctgggcgtggtggtgggcgcctgtaatcccagctactcaggaggctgaagcagggagaattgcttgaacccaggaggcagaggttgcagtgagctgagatcacgccactgcactccagcctgggcgaaagagcgagactccatctcaaaaataataataaataaaataagatgccggctgggcgcggtggctcccgcctgtaatcccagcactttgggaggctgaggtgggtgcatcacctgagatcaggaatttgaaatgagcctggccaacatggtgaaaccccgtctctactaaaaatgcaaaaattagccaggtgtggtggcacacacttgtaatcccagctactggggaggccgagacaggagaattgcttgaactcaggagacaggagcttgcagtgagccaagatcgtgccattgcactccagcctgggcaacaagagggaaactctgtctcaaaaaataaataaaataaaataaataaaataaaataaaataaaataaaataaaataaaataaaataaaataagccttctgtggtgttgttgaagtatgcttttttttttctgtcactaattcttaaaaatttcacaaaccattttgtaaagcatttattggacccttactgtttgccaagcactgggccaagagctttataaatgctgcctcatttaataccctatacaaccatgtgaagccctgggatcatacaaatgcttatatcctcctaaaattcatatgttgaaacaaacccccaatgcaatagtaacaagagttggggcctttgaagtgagattaggtcatgagggtaaagccctcatggatggaattattgcccttccataagaggcctgaaagagagcttgtttgcccttctaccctgtgaggacacaaccagaaggtgctgtctgtaaagcagagagtgagccctcaccagacactgaatctgctgatgccttgatcttggacttccaagcctccagaactgtaaacaataaatttctgttgtttataaattgcctagtctaaggtatttgtatggcagctcaaagagactaagacagctgcatacaattatttttcccatttgtatagacaagggaactgagacttagtgaggtcaggtaatgaatgtatccaggaccacacagctgcagagaaggagaggtggaatttgaacataggttgcctgactgtcttctcaacttattttctgctgaaggtgtgtggaggaccagatcgcagggcatatttaagatattctactcagttctcatcattatggaagggaataaccttctttttaaaaaaaatctttttatttatttatagggtttttgttttgttttgttttcgtttttgtttttgtttttgagccacggtctctgtcgcccaggctggagtgcagtggcacaatctcagctcactgcaacctctgcctcctgggtttcaagcaattctcccagccaccaagcctgatctctatggaagagaataacctaatcttgctctctctctccactttttggatactcagaataattttgattaagtgaaacctaaaaagaaacacctcatcagtcaagaaagtccacgtagaaactagacagattttatgaacacattgccaggcctagagttaccagctacatgccattggcaagaggcgtacacctttcattctgaacaggagttaaacagaggcatattctggtaccaaagaggtgatgaaaactctgatatcagggatgggtggaattgattgaagagatgcaatatgctaagtactgtgctctgtacttcatgtgcatgacatcttcaattctcaacttaaccttgtgagggaactatcgttattgttcccattttacagatggcgtggggagctcacattacctctccaaagtcacttgactaagaagtaaaggagcagggattctaacagggatttctctggctacaccactggagactaaacctctaccccttgcctatagggtaaagtagaaaagccatgagagcaagacaactagagtctcattgctaagtcaactttaggaatgtgaaggtgagggcagtatggcagcaaactttgcacttcaagcacacagaaaaaccaggcagtgccagattgacagagatagcctagtgtcttgaggatcttcactatctcccagaacctggaaggatctcctgaactgcttatcctggttgtttggttaattcattctttcttttatttttaatataaccctttattaagtataattcatctaccatacaattcaccttatttaaagtatatcattccttgagttttagcacccatcactacaatcaatattgaacattttcatcatgccaaaaaaaaaatcacatccatgagcagtcacttcctatttcccctcaatcttcccagtctatggcagccactatttgctttccgtttctgttgatttgcctacactggacacttcatgtaaaaggaatcatataatatgtggtcttttgtgactggcttctttcatttagtataatgtcttcaaggttatctatggtgtaacatgtatcaggctggagtgcaatggtgtgatctcggctcactacaacctctgcctcctgggttcaagtgattctcctgtctcagcctcctgagtagctgggattacaggtgcacgccaccacgcccagctgattttttttgtattttagtagagactgggtttcaccatgttgcccaggctggtatggaactcctgagctcaggcaaccctcctgccttggcctcccaaagtgctaggattacaggcataagccaacgcgcctggccctttattcctttttttaataatgttccattatttggaacacattttatttatttattccttggttgatgaactttttggttgtttccacttttcagctattatgaattattcttctattaatatttgtgtacacgttttgtgtagacatatttttatttctcttgggcatatatttaggaatagaattcctgggtctcttggtaactctatgtttaatcttttgttcaactgccataatgttttccaaagcagccacaccattttacattcccaacaacagtgtataagggttccagtatctccacattcttgccaacctttgtttttatcttggttattgccatcctagtggatgcaggttaattctttcaagcctgacttacctgccttctgaggttcctgatagtaaaactgcattgcaaattccagacagatgaaccacaatttgaaaaaatatatttgttggatacaattttaaacttgctaacccccacctaatttcctatttagtatcataaaaatattttctgaatattttatatgactatgtattatatatttcttttaaaatcctttaatgtgttcaaaattatgtatgtttacatttatttacacatgacttttgaggaacacattcactttataaagtaaggaatatttgtagtagttatgagtactaagattgttctcagtactcaaaaagtcactctgaaagtattaagaatgagtttactaaagagaatgctttcagaaatattcattgggaagagcatgggctttagagttttagatttaatttccagttctgaaacttaccagtcaggtgtcttttatgatacaaatatcagaaaactcaagtgctggccaggcgcagtggcacacgcttgtaatcccagcactttgggaggctgaggcagatggatcacttgaggtccggagttcaagaccagcctggccaatatggtgaaaccccatctctactaagaatacaaaaattagcagggcatgtaatcccagctacttgggaggctgaggcaggagaatcgcttgaacctgggaggtgaaggttgcagtgagctgagttcatgccattgcactccagcctgggctacagagtgagactccatctcaaaaaacaaaaaaataaaagaaaaataaaaaaactcaagtgcagtcacttaaatagacatggacttatttatatcaggtcataattatatgtagtaacagatggcatgataattcagtaataccccagggatctaggctcttttcctctttctactctgccatcttcagtgtgttgaccttaatcttcagatgctgtatctccaggcttcttatccaagttccaggcaggaaaagtggtaaagagcaaaggagttcctcctgtggacttcgacctacactgagttggccacaacagtgtcacatgtctactctgtctggatgggaacctggcagaagggcattgtaaatggagttggataccgtcatccaacagtatctgccatagttgctagctgtgtaatcttagaagagttatttagccttgctgagcctcagcctcctgtgaaatgaggataattaatgccacaaagcttataaaaaagcaaattataggaagtgacttgcatataggaaatgcttagaattaagtacaacacaaaatgatgtcttagtcccttcaggtgactatgacaaagcacctagactgcgtaatttgtaaaaaacagaaattcgttgctcacagttctggaggctaggaagtcccagatcaagtgtcaacagattttgaaggtctgttcttcatagatggtgacttctatatgttctcacatagtagaaggggccaggcagctcccttcaacctcttttataggggcactaaattgattcatgagggaaaagccctcatgacttaaccactttctaaaggccccacctcttaatactattacattggggtttaagtttcaacatgtaaattttgagggtacactaatattcagactatagcaaaggatattataatattatggcctaagggaaacctgaccgatttgattgagggaaatattaatgtaaagccataaaaattattatttataggatattatagataattttattcatatatttttaagtaatacaataattttggaaaaatctagaaagagatgttgcatgtgctctaatagaaagacttattttttgtatgtcaatagtacatttattcattcaaactagaatgtaggcttaatgtgggcaagaatttttgtttgattttttcattgctatgttgattgaatctagaatggggcctgacacatagtaaatgctcaataaatatttgttgagtaattattctagttgataactagaaatagacatttcctgaatctccccttcactgtctagttaagtaataatataaggtcatatatattttataaattatttttcccatcttctttcttaaaatccaacaataaactataaccaaactctgataaacctaaccaagttctgaattgatggaggccagattaatgggattttattacattctaaacaggacataaaagatataaaaggttagaataaaagtgaatgttctcctctagaaaagtcaaaggactaacttataaatatagacaggtcagaaaaggaaacaaacacctatttatctaatgattagttacaaacaaactaatgagttgaaaaaaattagagaagagggctgggcgcagtggctcatgcctgtaatcccagcactttgggaggctgaggcaggtggattgcttgagctcaggagtttgagaccaacctgggcaacatggcgaaaaatacaaaaaatataaaaatttctacaaaaaatacaaaaactagccaggcgtgttggcgggtgcctggagtccaagctacttgggaagatggcttgaacccaggaggcagaggttgcagtgagccactgtactccagcctgggcgacttagccagaccctgtctcaaaaaaaaagaaagaaaagagcaagtcaatttccctaactaccagaaaaaatctttccctcaaggagaaagctgacttataatgaatgttgtggtcgaggtagtattatgtttaaaggagctcttctaaacgttcagttattagggtattttccacattggtaactaggcagagatcagcatgtgcaaggtttcacttctaactcatgtggttcagttttccagtaaaatcacatgtagagagaagctttttacctgagaccacctactcatttctccagttgcagaaaggcagtgtttcccctatggcaacataggtgccatcacaggcctcctcatgttggggtgaagttgtaaagtagaattttgtctgttttgcttcaagagctttctttcttcccagctcctttgcggtggcctcaccagacagagcctcctggctgttcacataccttcttttacctcctgtattttgctgtttcaaagggtaatgatagggaaaaaaaaaaaccaaaaaaaaaacaaaaccagtgctaataaccatctccctactgtagtcccactatcttcagcaatttcatgtaaatatattttttatttatcttggaaacactggtaatgtctggctcagtgtattctactccataaatatttgttgaataaaaatgcatgaatgatagccctgcattctgttccattcaagtcatcttagcaaatatttatttagcatttgatggtttcctggaattgtattaaactgtagatatttaaaaaaaagagatcatctctgtgcatgacaagctcataaacagtttagaataacatcaatgttgggattttcacgcaccgtagaagaccaataaggcaaatgttttcagatgcagtatcatgttagagcccttaaattcagcggcctactctgctaattcataaggcctgtttgtttttgccacagcttctctacatgcatatttttaataaccagaaagagttcttaggtgtgattccaaatactatttttaaggctacgttaggtttcagtaattcctttaggactcaaaatgtagtttatattttagaatccatttcctcactgattatgagcccagagagctaattgtggctggagacaaacagaaccttgctgactgatactgctttatattcatgaccactaacctcaactgggcccggagagccacacagtaatcctactacatttcactagccaagtcactgtcttgctaagccaggtcattgcttcattactttttctgtcatctctcctctctagtatcttcccttcctcactctctattgaagacctgcttcctactttcctgagaaaacagaagcaatgagaaataccacagcctccctcattgactaatgcattgacatctgtacccatatagttgatctttcctgctattgctatggatgaattaccattgccctaaggtcaactctccacttgggtactagatcttatcctgcctgcctactggaggacactgctccaaaactttcctctcttccttctgcattgtcattgtctctgtctttactggatcattcccattggcttttaagcatgctgtagcctttgcccttttgaaaaatataaccctccctttgttcctgacctctaccacccaatttctctgtttctttttatgaaaaaaaatcaaaagagttgtctgcactgtctgtctctattttctctcttctcttttatctagtttagtcatgttttatgtcctcaccactccactgaaacagctcctgtcaaagtcactgatgatttccccattgccagattgaatggcttattctcagtctttatcttacttatgaaacatttactatccgagcatttaatgtagctcttcacttcctccatttggcttccaggacaggactcccttttgctgctcctcccaccttcctggaaactttatctcagtctcttttgctgattcatcttcatttcagataaattcatagtgacccagagctctatattttgtcagcctaaaaaaagacactagggaagattatcttcaaatttgttgggtttacttgagaatataaataaggagtataatacagaatgcatgacatggcaagccagcagtgcatttggtgagggaaaggataaagggaagcttttattaccaaaaaggaatatatgtgagctatttaggaacagagttcattagttccagaggctcaaagccagagtgattgtcaattaataagtggagatgtattactgggtaagcggtcttccaaaaacatcttatctggattactgcagtcctaaaggatgtctagtgataaaccttatcaaagcaggggatgactgaaaggtttttagaaagtccttgggaacagttcttatctcagacatgtaagcatgggcctcttctccttcaggcctttcttgccctattttgtctgggtctgacaaaagtgatttcatcctggtatgtgcaactttcacacctaggaccttttattatctctatttacagtcattacctaaaggatagccttcaaatttgtggtttaaaattcctcaatgtatatctgtgtctaaatatctccaccaaatacagctgccatgtttctaacctgtcattcaaggtatcttgaagttaacattttcaaaaccaaacacccagtttcctctcccaagacttgcattttccatttcagtaaatggcaactgtatcctttgcattattcaggtcaaaatcaggtagattctcctggattcctctctattatggccttcatctaattgatcagcaaaaccttccggctctatcttttaaaatgacctcaccacctacactgcaatccaagccagatcatctctcacttgggagtataagaacttctaactggtccactcgcttcctccagagtcccttattcatgagttttcacagtaatccttttcacatgcacctcaggccatgtcacccttctgctcatgaacctcccatttcattcagagatcaaagccagagtccctcaaggactctatgtgaccttgcaagtcttctaaactttcccctcttccaaaactctctgattttgtctttaatccctttcctagtaaaacctcactgacctccttgctgctctttgaacaggccaagcaaggtcctgcctcagggcctttgcatgttacctagaatgctcttcctccagagagccacagggtttccttattgccctaatgtcaccctctcagataagactttcctcatctctctaaaaaacaaaaaccttaaaacccacccctactctcagaactgcctaacctttttactccgctttagttttcttctgaacatttctcaccatacatagatcagattctatatttatttgtttccaccaaaatgtgtgcttcatgacagttgggactttatctgttttgttcattgtagccccaatatctaaaaatatacttggaatatagtcagtgttaaataaatatttattgaatgagtgattaaatgaatgaaagacactatttaccattatcaacataaatctgcaatgttttttgaaaatctataaattacaactatatctaggtgctcagggaacatcagaaaaatatgaattatactaactcccttcacaaatcatctgtcatgtattagcccatgaaaaaaagaaatagaaataagatattcaaaaactttgtaatatacagttatgtaaaagatagaggaaaaaccaaagtgttgttctcctttctactctcaacagcactcctgacacgaaatgtgggaagattttccccacacaccaatcaattctcctgcagacatcagctagatgtcctctaatttaattcaattctgactctgtctacctggaggtagcatcagatcccacagctaaaggctcagtcccacaagactcaaccctgttttaaatgtcaatcacaagccccaggtcatgacctgtgcttttgaccaaccggctaaaaattgtggtttccacgatacactcctcaggtttgattaacttgctagagtgtctcatagaactcaggaagacactttacttatatttatccatttattataaagaatataacaaaggctacagatgaacagccagatggaagagatgcatggggcaaagcatgcgggaacaccaccctgcaggcatcctcatatgttcagatatctggaagctccctgaacccagtccttttgggttcttatggaggcttcattacataagcacggttcatcatatcattggccattggggatcattcagcccttctctcttcccaggagatgggggtgggtaaagggcaagggggttggggggactgaaagtttcaatcttctcatcacattgttggttcccctggcaaccagcccctaccctaagcctatccaggagcccccagccatcagccatctcattagcactttggagattccaagggttttaggagctgtgtgctgggaaccagacagaaaccaaatatatatttcttattatgtcacaatatcacaagttctatataaaaataaccctaaggtgtaattagtcattattatcagattcttcaattaaaacagtgctttgtggtctaggtgctaatatggggtagactttaaaagcagaattaaattacttagtaactatggttactgtcagtttgaaaatacatttccattttgacttgtggtctcagtatgtgtggtaaaagaattagccacaaaccaatgaagaacatataatgtggttttctctaactggaaaatattcaaagatgtcctacttaccatttacacatagttgccattttcttgttttcacatttgaattcttacttagcatgaggttaacatactttaacaagttctctgttaaggtatttacgtttcatcttatttcagaaagaatataaggtaactatgtaaacatctcaatttaacggcataatttaacttaagatacagtgatataagagaaattttactggtaagaaatatagcaattttaaaagtttcatttgtattctttagaatgtgtcaaagtcacatatcataaatatcttctttttttatttaaatggacacatagtctcactctgtcatccaggctggagtgcagtggtgcaatgttggctcactgcagcctccatctcctgggctcaggcaatcctcagcttcctgagtagctgggaggacaggtgcctgccaccacacctggctaatttttgtatttttagtagagacagggtttcaccacgttggccaggctagtctcaaactcctgacctcaagtgatctgcctgcctcagcctcccaaagtgctgggattacaggtgtgagccaccatgcctggcctcagccagtatctcttaattcagaaaaattgttgtttaatgagtccttcattaggtgagaagccatctactctgtttcttatttaatttactcatgataacaacaacgttatcattcctatactgaggatgagtaaactgagctgtatagagcttaagtaacttgctccaagtcttacaacttatcaaggggcagagctgggatttgaaggtaggtctgtctctaaaatccaagccctgtgattttagtcaccatgacataccctcctcgtcctctcatggtgttcatagtgatctcacgctggtatgaggaaagaggtaacacagaaaaaatgcactggaaccgaaaaacctgagctgaataggaggagttgctcctcacccctccaatgactatcagccttagagagattccttgaccacttgctgaggttgtcttgttctgatagtagcaataagtttatataaattatataaaatatagaatttctatagtacttagatgcacacatctgcttattgcttggtattaatagcataaggctggttccttttctctgcgtctcagcaagcacgtttcttggattccaaaagatgtaagtggctagcaagtttaatcctggtgggatattttggggctagaaaaatttaggagttgctgaaaagatatatagtagtaacaggacttggtggagccttggtggagggacagtgatttttaagctttatcttctccacaaatccaatcaaggcagtcccctgagagagagggcagttgatggtattagatttaaaaaatcaggttagagtctgcctcaacagcacaaacagtgtaattggaacaagacaaagaagatggcatgacccctgatcaaggacggcatgcaaatttgtgaagtatttccatttgatacaaataagaatgtatatggctgagcacggtggctcatgcttgtaatcctcacgtgggaggatcccttgaggccaagaatttgagaacagcctgggcaacatagcgaggctgcatctcttaaaaaaaaattattttaaataaaaaaattagttaggcctggtggcacatgcctgcgatcccagctactcaggaggctgaggtgggaggatcacttgagcccagaaagtcaaggctgcagtgagttatgatttgtgccactgcactcaaacctgggtgacagagtgagaccctgtctctaaaaaatagaagaaaataagaatgtaggctacttggctttccagcattgttcagatttcaaggttactttccttctaaagtagtatatctatatcaagttaagtttcatgatgtaatgcaattaacttaggagtctgaaagtcataaaccaaacaggtaacaaagacctagaccattgccatagctatcaaaacagggaaaaaagataacataggaaaataatattttaatgcttaaaaaaatgcttaaccttctttagtaattggttggctcttaaaggtgacgaagagaaaattgttaaaaatggcaaactgagaaaggtggaccgcatgcctttgacaacaatgaagacactggcaagaagtgctgatttgcatggaaagataatttagttgtcagttttctatagttctggttccagtcagacatccaaatgaagctcccagtgagggcacaggtatgcgtgagacacacatgtgaaatagcaggtgaagctgagaagggagtgagcccagaaaaaggggtcagggaaaaggcaaacaccagggctgaccttcaatgtgagtgaactcagagagtagggaaagaaacccaggtggaaggctggagggggacatgccactaagtaggtgtgagagaggaaggccctctaacccagaagacaggctttgggtagcaaggaagtgagaattataaccaatcaagagggataaatgcttaaatgaggattcagaggacttcaccttgtaaaggacaccattttaaaagagcagtttcaaggaaaagtaaaggcaaagatcacaataaaaggactaaagaccaaatgggaataagttggatgtagttactaaggaggtagaaaatctgagaagcttaagaacatgaaataatggagtaggtagttcaaggggctcaagaaatgggaactgaggaaattctagtgaaaatacttccatgttacaaatgaatctgattacagattatgttgtgttttggagtctgaggggggccctattcatctctgcagaagaatgagtgaacaaggggtgggacaagaggctaggagatttgaggacactcctgtgttagtccattcttgccttgctataaagaaatatctagaccaggtgtggtggctcacacctgtaatccctgaactttgggaggccaaggcaagaggatcacttgagctcagagctcgaggctggcctgggcaatgtggcaaaaccctgtctctacaaaaaattagctgagagtggtggtgcatgcctgtagtcccagctactcaggaggctgagatgggaggacctcctgagcctgggaggtcaagactgcagtgagctgtggccataccactgcactccagcctgggtgacagagtgagaccctgtcattaaaaaagaaaagaaaagaaaagaaaagaaaagaaatacctgaggctcggtaatttatgaagaaaagagttttatttcagctcacagttctgcaggctgtccaggaagattgtgaacaagatagagggaggggggatatgccacactctttaaaatgaccagatctgcctgaactcagagggagaagtcactcattactgccaggagggcagcaagccattcatgaagggtctgccccacaagtcctcacctccaacactggtgatgactttgcaacatgagatttggagaggatgagggtggaaacaatatcaaatctcttcacagaaatcagaggagcaaagaagcattctccagcactcctttcgccaccgcctccaaccctcgtccactaaaaacatcatgtgattatttaggggtagagatttaaaaacagaggctcaaacattctgtccaaagccagtgaaaacaatttgaaactctggcattaacagatatcattcggaattctaccaagtttattgggctacttggatattctcattcaattataaatacttagataaatatgctttgaaatgatacttaatagccctctctattcccctgttctaaactagatctttcctctttatcttttccatcctcttccacagtgcatgactccctttcccctcatcaaacctgtcattaggtccagtcagttctacactcaaaatccttcatgggttgggcgaggtggctcatgcctataatcccagcactttgggaggccaagacgggcagagtgcttgagttcaagagttggagactagcctgggtatcatgggaaaaccccatgtctacacaaaatacaaaaattaattgggcatggtagtacgtgcctgtggtcccggctactcaggaggctggggtgggaggatcactcgagcccaggaggtcaaggttgcagtgagccaagatggccccactgcacgccagcctgggtgacagagccagaccctgtctcaaaatccttcatatccacccccattggcttccacctatttcagactaataatccaattctggattattgtatagccttttgatttttccactaccaccactttcatttcccacaaatcctccgtaaaggtgctagacagtcccagagaccctcaaaatagtgtgaatctcactatatgcactcactttctatcacttacatgatacagtcctgatccatcagtagctcatggaaggtcttctgacaggcctgctccactacaagtttcttcttgcattttcaggatccaggctattgcacttgttgaaccttctcagaaatgtcatgttatttcacgtatttgtttcagaacatgctggttgtctctgtccaggaggttggccttccccctaccccattgcgatctctcaccccatcctgactcctccccaccactccatcctcaccctatgtcctgatagaaatgtctgacttcacagtccttgcactgaaccaaaatgcagtgctcttttccaaaggcagctattctggctattccaagctatgtccagtgccgacttccctaacaggcacagtaggcacagtggctagggcccacaataattttaggagtccatgaaaatgtttaattttacttaaaatcagaagagaaaaataactgttatgttcatgtatatgcatgtatatggcatgcatatgtatacatctatgtatatgtgtgtatatttttgtgtattttatatatttatataatatatacgctcattgtttttttaatggaggaaagggtccatgaaggcagaagtgttcagggcccattgaaatcatactgtggcccttgtcagatttgaatttattttttaaaattgtgaaaataaaatcaattttgaaatatttttcagtaaaaggttgcatatttgaaagcaggcttaaaaagttagcgtttttgtccaggcacagtggctcatgcctgtaatcctagcactttagaaggctgaaatgcacggattgcttgagtccaggagtttgagaccagcctgagcaacacagcaagaccctgtctctactgtttagaaaattaaattttaattttttgtttttgtttttgtttttgagacggagtctcgctctgtcgcccctgctggagtgcagtggtgtgatctcggctcactgcaagctcctcctcctgggttcacgccattctcctgcctcagcctccccagtagctgggactacaggcacctgccaccacgcccggctaatttctttttgtatttttggtagagacagggtttcaccatgttagccaggatagtctcgatctcctgaccttgtgatccgcccgcctcggcctcccaaagtgctgggattacaagcgtgagccataaattctaattttttttaaaaaggtaacattttaaaaatctggaataattaattcaaactttttttcttttttttttttttgagacagagtctcactctgttgcccaggctggagtgaatggcgtgaccttggctcactgcaggctctgcctcccgggttccagcaattctcctttcgcagcctcccaagtaactgggattacaggcacatgccaccaattattgtatttttttaatagagacagggtttcaccatgttggccaggctggtctcgaactcctgacctcaggtgatccaacccgcttggtctcccaaaatgctaggattacaggtgtgagccaccatgcctggccaattaattcaaactaaatacaatttatgatcatcttttttttttgttttaagtttagagacagaagaagagcaacagtatctcacctcaagctatattaacatctccactgattcattacaaggtggcaagaagtacttggtttgggtccaagcagcaaacgcactaggcatggaagagtcaaaacaactgcaaattcacctggatgatataggtaaagaataagaaattctgtaagtctttaataataaccagtttgtgctgaccttgtcaaatgaggtctagatctgaaagaagttcccctaaagttcctgcagagcatgataaaagaaacagaaattacccataattctacctgcccaagacaacttagagatgagtagtgctgctgccattttgttgtatatccttccagtcttatttctaaattgttgtatgtggctgggccggtggctcacgcctgtaatcccagcattttgggaggtcgaggtgggcgatcacttgaggtcaggatttcgagaccagcctggttaacatggtgaaaccctgtctctactaaaagtacaaaaaaaaaattagccaggcatggtggcgggcacctgtagtcccagctactcaggaggctgaggcaggagaattgcttgaatctgggaggcagaggttgcagtgagccaagattgtgccactgcactccagcctgggtgacacagcaaaactcttgtctaaaaaaaaaaaaaaaaatccatatatgtgtttttacaaatatttattggaaacttcttattttcaagcattgttctaaaggctagagtgaataagatagacaaggtgcctgccctgaaataacttacaatctactagggaaggcaaaaaaaaaaaaaacaaaaaaacactatgatcatgataaagtttgttgagtcttacgttgtgaaaagtacagcatgctaaattcttatactctatacactacttggggacttatgtaaggtttttttgtttgtttgtttttattttgaaaagttttacaaaatacatagaaaattgcagagaattatgtagacctctctatgttcatcagccagatttaacaaacattaatagtttttgtttcagatattttataccctgcctttttatacttatatattagtaataaacatttctattaatttaatctatagttttatttttaatgttgtctgaatatatagcagtttaatccctattgttggagattaacactgttttcagtttttaaccatataaactatattgccatgaacaactttgtggctgaattttagcatagtttttgattgtttccttaagataaattccaaaaagtaattacccttgagccttttacaactacatccaagtcctaaaaccacaattcagccaaatgacaccaagtctgaatcatcctgtctttgcagatcccagtaccaatgattcctgttgttataattaaagatatttcaataagctaacatttattgagcactttcttatgtgctaactactttttctttttttgaggcagagtctcactctgtggcccaggctggagtgcagttgcacaatctcggctcactgcatcctctgcctcctggcttcaagcaattctcatgcctcagcctccctagtaactgggactacaggtgtgtgccaccatgcccagctgatctatattgccacaaagttgttcatggctatatagatttttgtatttttagtagagatgggattttgctatgttggccaggctggtctcaaactcctgagttcaggcaatcctcccacctcagcctcccaaagtgttaggattacaggcgtgagccactgccagctaacagcttttcacatacgttatttaaccctcatatcaaccctattttgtagatgagattataatttatatattagaaaactgaggtttgatggcattaaaaaatttgagatcacagaggtagggaacatttaattcaggggatcctcctgccttagcctctcgagtagctgggacgacaggccacaggccacagtgcctggctaatttttttaaatctcttttagagacagagtctcattttgttgcccaaacttgtctcgaactctgtggctcaagcgatcctcctgccttggcctcacaaaatgctggtatttcaggcgtgagccaccatgaccggcctgaatattagatttgaaaacacactctgttgaatatgatgatggacaattattccattttattgtatatccttggagttacagttctcttgactatgctctatacatttttgtgaatttacattttgaggtagaatgcagtaatatacccttttcttacgatagataaattaatgatgtttaagagctatcatgaccagtatcattttagcagtgcaatttttaggaactaaataataataataatacttgtattattattaccattctgatcttaatatcattgcatcaatttatttagcaccttctgtgttccagacacctctcatgcacatcacgtttcattctggaaacaaccatacatgatactactgtccccattttacagatgaagaaagtgataaaatgatttgcgcaaagtcacttagctagtaatgaggaaagccagatttcatcccagacatgctgacttgctatagtttaccaccatcccacctcccacaaaggaaaaaatcccatctggcctctacttccagactgacttctcacactgggaactaggagtttggagttgtggttccttttccttcttctccttctttctctttccctcctcctcttcctccacctcctcctcctccacctcttcctccccttttgtctctattcctttccttgccttttcattatcccagaatgtactcagaaactcaggttgaaaaatatggattaattgtataggaggccccagccaacaaaaccaccagattattaacagatgagccgtcttcatgggtaacactttaagaatgagacaacgcaaattagataaatgaactgttctatgccactgagaacagaagactgtcctgggaacaatttccacatagcagctacgggctgggctttaatgacattagggaggttatgctgaatggcagcgcagagttagtagcacattaacacttgccaaaaaagcatatgagaggaatgcttttaacttctcttgtagggaaataaattcgggtacagaaattgctgtgctttctgcctgccattttgaatattacttctccccattatataaatatggataaatatgtgttggaaagatgctaaaaggctataatctcgcttttatttcaatacgagtagaatttaaaaatatgacatctataataaaattgatactttttttttctttttttgagacggagtctcgctctgtcgcccaggctggagtgcagtggcgcgatctcggctcactgcaagctccgccttccgggttcacgccattctcctgcctcagcctcccgagtagctgggactacaggcgccagccacctcgccccgctaattttttgtatttttagtagagatggggtttcaccatgttagccaggatggtctcgatctcctgacctcatgatcggcccgcctcgacctcccgatacttttttaaaatgtaagatctggtggaaatatgtgaaacctaatcagtaagaataaaaattcaacatctgagtcttgtgtaacaaactaaatattcaagtatgtattacataataaatgccagtttgcaaaaatatgaactcattcaaacttaaagacttaaatctggattgcactgacctgctttatgctgtgattcttactgtgctatctgcatttctcataagactaaacagatttttactttctcctcagaataattcttttgccaaaagggtcctagaatgcttgtgctgctataacagtggccaagccgaactcagaggtggaggtgggtgacaggaggaggtgagaaatttatggccttgagggtgcttaaaatggtcctaagcaatttcagagccccagggatccatgcccttgactctagaatctcaatactgattttttttttttttccagggttggggttgggcagagtcttgctctgttacccaggctggagtgcagtggcatgatctcagctcgctgcaacctctacttcttggattcaagtgattcttgtgcttcagcctcccaagtagctgggattacaggcgtctgccacaacacctggctaagttttgtatttttagtagagacaggtgtttcgctatgttggccaggctggtcttgaactcctgacctcaaatgatctgcccacctaggcctccccaaagtgttgggattacaggtgtgagccactgcgtccagcctgaatactgattcttattacaagaagcttgcactagcaaaaggaaatcagctatggaattcagatgataaaggttgagtacagtaaacttgttaactaaattaaagctactgctacttctacgccaggtgtagcccccaaaattttattagaggactgtgttactagacagcaaatccttaatttttgtttttctctgtaaatcatctacctttgcacataattttagataataatagtataatagtcattgtagtaaaacaataattttcttaaagaaagttttataaacctagaataaatgaaaataaactaattcatagagaatattaacctgtaatagtggtctctaaaaccacgcctacaggcccttgagttctttgttaaatagtacagctatcaaagtcataggttttcattgttccttaggggagaaatggaaagtgttaatgattctattctgttcagacaaaacaaacttacaagtatttacacagtttagaaacatagcgattccaactatatacctcctactataacaaaacaactaataaaataaccaatcagaatttcagttccttctgtgaaggttattattatagaacatgccaacaatttacaaagccttagagtcttatgagatctgctttcttcaaataaaatttcaaggaaggacagatttattttaaaggagaacaaaacaagttagcaatagatataaatattcatatgtttccctaatacacaagagaaggagaagattattgtgataaaagatctgaattgaacatccatctttctttctttctttctttctttctttctttctttctttctttctttctttttctttctttctttctctttttttttctttctttctttcttgctctttctgtctcttcctttctttctcttttccttcctcctttcccttcctttctttcttttttgacagagtctcgctccgccacccaggctggagtgcagtggcatgatctcagctcactgtaacctctgcctcccgggttcaagcgatcctcctgcctcagcctcctgagtagctgggattggcacgcaccaccaccacgccgggctaattttttctgtttttagtagagacagggttttgccacgacgaccaggctggtctcaaactcctgaccttaagtgatctgcccacctcggcctcccaaatgctgggaatacagacatgagccaccatgcccagcctttctttcttttttttgagacaagctctcattctgtcgtccagggtggagtacagttgcaccatcatggctcactgcaaccttgaactcctgggctcaagtgaccctcccatctcagcctcctgagtagttggaatttcaggcacacatcaccacacctggctcaatttaacatcttttcaaatctgatcatctgaactaaatttgggtgcatatcatctctcctcgaacagatggagggaagaggggatagctcctttatctcatttttatactcttttgttaacaaggctaggctctggaattttattagttaggattgtaagggagtgaaaggtgggggaaagacaggactattagagttcagtttttttttttttaatggggaatgggctgaatagaattttccttaacagcagttttcttctatgtaacaaattagcaaataagaaaatgtccataattgttcattagaccatgaattttatttttttttactttgagctttctcatatctagatattgacgaaaagatgccagtttctccctaggcaagttttaaacagccaggtctttttttttttttttttttttctagtgataccttctgcagccgtcatttccagggctgagactataaatgctacagtgcccaagaccataatttattgggatagtcaaacaacaattgaaaaggtttcctgtgaaatgagatacaaggctacaacaaaccaaacttggaatgtaagctcaactttcattatgctttagcatgtgaatgaatgatttaaaagccaaccatcagtggctacagtggacttattatgtctattttacatgtttttaatctgattgtttgcatgatattcaagccactttagtttttgttttataatttcaacttttattttagattcgggggcacatgtgcaggtttgttacctggatatattgcatggtattgaggtttggggtatgattgatcctgtcacccaggtgctaagcatagttaccaataatttgcttttcaacccttgccttcctaccttcctccacactctagtgtggtccccagtgtctattattgccatctttatgtccatgagtacctgatatgatttggctgcatccccaccaaatcgcaacttgaattgtgtctcccagaattcccaggtgttgtgggagggacccagggggaggtaattgaatcatggaggccagtctttcccacactagtctcgtgatagtgaataagtctcacgagatctgatgcgtttatcagaggtttccgcttttgcttcttcctcattttctcttgccaccactaagtaagaagagccttttgcctcccaccatgattctgaggccttaccagccatgtggaactgtaagtccaattaaacctctttttcttcctagtcttgggtatgtctttatcattagcatgaaaatggattaatacagtaaattggtaccagtagagtggggcattgcttaaaagatacccgaaaatgtggaagcaactttggaactgggtaataggcagaaattggaacagtttgaagggctcagaagaagacaggaaaatgtgggaaatttggaacttcctggagacttgttgaatggctttgcccaaaatgctgatagcaatatggacaataaaatctaggctggggtggtctcagatggagatgaggaacttgttaggaactggaggaaaggtgactcttgttatgttttagcaaagagactggcagcattttgcccctgccctagagatctgtggaactttgaactcgaaagatgatttagggtgtctggtagaagaaatttctacgcagcaaagcattcaagtggtgatttgggtactattaaaggcattcagttttaaaagggaaacagaacataaaagttcagaaaatttgtggcctgactatgcaatagaaaagaaaaacccattctgggggggagaaattcaagccagctgcagaagtttgcacaagtagcaaggagcctaatgttaattccctagaccatggggaaaatgtctccaggccacatcagagacctttacagcagcccctcccatcacgggcttggaggcccaggagaaaaaagtggtttcatgggctgggaccagggtcactgtgctgtgtgcagcttagggacttggtgccctgtgtcccagctgctccaaccatggctcaaagggccaacatccagcttgggctgtggcttcagaaggtggaagccccaagctttgacagcttccacatggtgttgagcctgcgggtacacagaagtcagaattgaggtttgggaacctccacctagatttcagaagatgtatggaaatgcctggatggccaggtgaaagtttgctgcagggacggggccctcatggagaacctctggtagggcagtgtggaagcaaaatgtggggtctgagcctccacacagagtccctagtggggcattgcctagtggagctgtgagaagagggccaccatcctccagattccagaatggtagatccaccaacagcttgaaccgtgcacctggaaaagttgcagacactcaataccagcccatgaaagcagctgggagggagactgtaccctgcaaagtcacagggttggagctgcccaagaccatgggaacccacctcttttatcagcataacctggatgagagacatggaataaaagaagatcatttgggagctttaaagttgccctgctggattttggacttgcatgggcgctgtaacccctttgttttggccaatttctcccatttggaatggctgtattacctaatacctgtaccctcattgtatctaggaagtaactaacttgcttttgattttataggctcataggtggaagggacttgccttgtctcagatgagactttggactgtggacttttgggttaatgctgaaatgagttatgactttgggggacaattgggaaggcatagttggttttgaaatgtgaggacacgaatttggaggggccagggatggaatgacatggttttgctgtgcccccatcaaatctcaacttgaattgtatctcccagaattcccacatgttgtgggagggagccaggggaggtaattgaatcctgggagcccgtctttcccatactattctcatgatagtgaataagtctcatgagatctgatgcatttatcaggggtttccgcttttgctttttcctcattttctcttgctgctgccatgttaagtagtgcctttcaccacccatcatcattctgaggcctctccagccatatggagctataagtccaattaaacctctttttcttcccagtctccagtgtgtctttatcaacagcatgaaaacagactaatgcagtacccagtgtttagctcccactcataagtgagaacatgtggtatttgattttctgttcctgtgttaattcacttaggaaaaccactctagctttaataatgattattgtatgcttgcaaacagagaactgtttcctcaaacgatccacttgccttttattagttgctaaatagacaaagctccgactaagaggaatctaattagctatttgtaattcagtgtctcctagggtgaaatttatatcagtgatcatggataaaaaatttaagtattctgtgctgaaatttgacaggcatccagggaaacaaaaattgttcggaaaggagaactaagatgtatgaatgtttctttttaagtgaaaagtgtatagttcagagtgtaatatttattaccagtataggcctgagtcttaggtgagcttaaagatgatgataatccgagtaaaaatgagtttaaaaaagtgaacgtctagagaaacaaactaaccatccttaaatgccaaggttaaattatctagtgtcttaagaaaggcatagctgcaaggttcattaataaagtcacatcagataacagcacctgggaacaacgtaataaaactcagtaatttcagctgtggaggaacactagcctgatttaggaagaagttccaattttgatatattttaaaagaaatgctatttgattatttttaagcgtaacaaactgccatttagtccaaataattaagaaatagtttctggcctttttttttcccttccgatacttttttaaggttttttttttagagatagactctcacttcatcacccaggctgaagttcagtggtgcaatcatagctcactgcagccacaacctcttgggctcaagtgatcctcctgccttagcctcccaagtagctgggactacagtcactcatcactatgcctggctaatttatttttatttttatttttttgtagattcttgccatgttgcccaggccggtcttgaactagcctcaaataatcctccccctggctttctcgtcagcctcccaaagtgctaggattacaagcatgagccacagcacctggcctggcttttcttaattaacagttatgtatcagctgtgaaattaccagttttcagcgcgttaaataaagtgatatgtatttaacactaaaatacattaaacataacttaatttttctttggtgctaagcatgattccaaatccttttgctgtttaagactgattctgagtatcggttttgctatagagtaatatatcttaaaagtatcaagaagatgggggcaaaactatactaattttattatataccctaaaaattactcattaaaagtaaattccttacgtcaacttgtttacctttgttcactcaaatcataaatgtgaactttatggttgtttgcatacacttaaatgggatccacgttctgcatcatttgattgataatcaagtgaagatcctgctgaattccttttgcatatgcagaatttagattaaatttcaaaacaacacaaatacaattctcaagtcctagattctgaattaatggggttttatcctaataagacacctggggtccttgtatagtatcacagtcatagaatgatattaaagaatactgagtttcttaggctgggtgcagtggctcatgcctgtaatcccagcactttgggaggccaaggcaggcggatcacctgagctcagggattgaagaccagactggccatcatggcaaaaccccgtctctactgaaaatacaaaaaatttagccaagcctggtggtgtgtgcctgtaatcccagctactcagaaggctgaggcaagagaatcgcttgaatctgggaggtggaggttgcaatgagccaagatggagccactgcactccagcctgggtgacagagtgactctgtctccagaggaaaaaaaaaaaaaggataccaaatcctcttacttcatgcaaataggagtatgtaatagactagaaaaagtgtttagaaaatagaaaggaattatattatcagtgtctctgaataagttttcagaagccaactgttttctggttgaaactcttattctctgctccccctggtggtgctacataggccatcttggtaacaggtacatttgagctcacttttcaaaaccttctctttatcagaagtggcaaatagagtagaaagaatatttgttatctcatatgctcttcataacaatcctttgtgataggtagtattagctccattatataaatagggaaatagagtttgaaagaagtcaagccagatttttttgaacttataccgtcagtaactaagtgcctctcacagactctacatcactttaaagaccaaataaatatttagaaaatgaaaagacaggtttcaatccaaagccacttctgtctcctccaccattatttttttcagaaagtttttttaaactcatgactccctcagtcaatctaccacgtttcctttaaacacaaccactaacacagaaaaagtgaattaccatttctatccagatcactcaagccaagtcactgtagccagaatgaagctttgtttacatttgctactgtcaatttcatctgggtcatgcatgaagtgttgtctctgcatgtctgtaggaagacaggaaagtcagagtcaagaggagtgggaggataccaaagattacaggtctcttctaccactttagctccgtggtggcattgcctccattagatattgctggaggtggggggtttacttcaaagcgacaggaaagccttgtggccaagagcacaggctctagagttcaaagcctggctctgctactttctagtcatgcaatcttttttttttttttccttttgagacagggtcttgctctactgcccaggctggagcgcagtggcgcaatctcagctcactgcagccttgacctcctggactcaagcaatcctcctgcctcagtctcccaagtagctgaccacaggagagtgccaccatgcccagctaaattttaaaaatttttctgtagagacagggttgtgtcatgttgcccaggctagtgtcaaggtctctaactgctgagctcaagcaaccatcccgcctcagcctcccaaagtgctggtattacaggcacgagccactgctcctggctaagtcatgcaatctttctttttctttttttttctttttagacagagtctcactgtgttgcccaggctggagtgcagtggcgtgatcttggctcactgcaacctctgcttcccgggttcaagcgattctcctgactcagcctccctagtagctgggattacaggcatcgggcaccacgtctggcttctttttgtatttttagtagagatggggtttcaccacgttggccaggctgatcccaagtgatctgcccatctcggcctcccaaaatgctgagattacaggtgcgagccactgcgcccggcctagtcatgcaatcttgagcatgtttcttatcttctctgtgcctcgttattcccatacataacatggggataagataagcccttatatcatgtgtttgttgtgggaaaggggatccaatgatgtaagaagttagctacaagcctggaatagtgtaattgttcactaaatgctagctagtaatgttactctctagcctctaggggagccatgcagcagggatagtagagcaaaggaatccaactgaaagtcacagtattgagggtgcttttcagcccctttagctaaggtacatacagactgtgaagtatctaagggaattaggctgacgaggcaaggagaatatgtgccacgcagcagtccatgtttcaggcaatgatggatggcatatatgatggtggtcccataaacttatcatgcagctgaaaaatttctgtcacctagtgatttatattacaattgcctgcaggattcagtacagtaacatgctttataggtttgtagcctagaagcaacaggctataccatatagcctaggtgtgtagtaggctataccacccaggtttgtgtaattacaatctatgatgttcgtacaatgatgaaatcgcttaatgatgcatttctcagaatgtatccctgtcgttaagtgacacatgactctagttatctcttgaaagatggttccaggtcaaagcttcagattttgtctctggagacacagctgcctctgggagaacatttacaaacatcttgctcccacctcatttacctctctcccttctgaattccctagaagtctagcttagaccaggttggaatatatacagattttagtaaaggctcagagatagcaagaagaaaataaatctcagctgggaggggtgatacaagcctatattcagctacctgagagactgaggcaggaagatcacttgagccagggttttgaggctgtaatgcactatgatcccaccagggatagccactgcactgcagccggggcaatacagtgagacccccatctctataaaaagaaagaaaataaacatctcagagaaaaaccacctactatgtaatcttgattacaaaatataactagcttttcaaagttggtttgagacctgtcttttcattttctaaatatttatgtggtctttaaagtgatgtagagtctgtgagaggcactcagttactgatggtataagttcaaaaaaatctggcttgacctctttcaaactctgtttccctatttatataatggagctaatactacctgtcacaaaggattgttatgaagaccatatgagataacaaatattctttcagttccatttgccacttctgacaaagagaaggttttgaaaagtgagctcaaatgtacctgttaccaagatggcctatgtagcaccaccagggggagcagagaatgagagtttcaaccagaaaacagttggtttctgaaaacttattcagagacactggtaatataattcctttctattttctaaacatttttctagtctattacatactcctatttacatgaaataagagggattggtatatcggctatctactgctacgattgagggaagagagagaccctctcatattgttttatatttttttatactcagtacctgttttaagaaaaaacaacaaggaagtaaaagcaaagacaggcaacccagcaccaggcccgaaaccaggactgggcctgcctggccaaaacccagtagttaaaaatcaactcataacttagaaagcgatgttattcatagattccagacattgtatagaagaacattgtgaaactccctgccctgttttgtttctctctgaccactggtgcatgcagcctctgtcacgtaccgcctgcttgctcaaatcaatcacgaccctttcatgtgaaatctgtagtgttgtgagccctttaaaaggacagaaattgtgcattcggggagctcggattttaaggcggtagattgccgatgctcccagctgaataaagcccttccttctataactcggtgtctgagaggttttgtctgcggctcgtcctgctacacaataatgtgtaacaaactgccacaaaagctgaagggacatataacaataaacatttatttctcaatgtcagtggggtccagctggtctaaggtgagctttgctggaattgggcatgtgtttgcagctcagctggctcactcctatgtctttgcatgagctctcctctacatgtctcctggtcctctttctgccatcaacaggctagcctgggcatgtccttatggtgatggcagagggcaagagtacctaaggcccaatctttccagtgtctttcaatcctctgcttatatcaggcttaccaatgtctgttggctcaaagcaagctacatggacaaaccagagccagaatgggagaacgttcaaagctacatggtaaagggccaggatatggggctgggaaattacctgggcattattgcaatccacctcaaaatgtcatcaaggctctccacctgctgctggaaccagttcgtgtgaggagaggaagatggtagcatctttccaaacaataaagaccaagaaccttggcagtcttcatcctactctagcatttgacactgttgaccaccttgtcctgaaaatgtctctcctcttggttttcccatttcaagaatgtgttagtcttctatcaatatttctggatgccctttcctttgtaaattattttttctgccaattccctaaacattgacattcccttcatacctaccatctcactgtctcctctctttcttactcctttcacctattcccaaggcctcagctacacctgtatgtaggctagaaactggagccctcccaactctcatgaaagtcagacctatatttctaactatttgtttgtatctttatgggctgtctcctaggcccctcaaattcatgtcaaaaattgagctcccagttgcgaacggcaatccacacctacttccgtatactctgtttcagggcatttgccacattgtatatacttgcctgtttacacttctatttctctactctttaactgtgaactcatggaaggcagagatgtgccatgatcattttttgcatctccattgatttttataatgtcaagttcatgtggataatattttgaaacattagctattactattattattattatttgtctaatggtgtccaactttagtgacagaggcagggaaatctcagctatgtgttcaagtaagtggattattggtgactcatttctctaattaagctacaccagggtctcagcaaactttttattcaagggccaaatggtaaacattttaggctttgcagaacataagatttctgtctcaactactcaactctgtgtttgtagcgtgaaagcagtcacagacaattatgtaaatgaatgggcatagtctgttccaattaaaatttcatttttggacactgaaatttgaatttcacataatttccatacatcatgaaatagtctttttttaaaaaaaatttctttcaaccatttaaaagtgagaaaaccattcttaacttaggctgcacaaaaccaagtatttattcttttcttgaaagggctgatagaacagcatacagatagcaaagacaaatccttggcttcggcatttccaagtctgaaggttggtataagacagaaaatgtggaaaaatgtatgattagtcacaaataagtttccccatcaccaccccatttccctcctctaccaaacactagataaaaattacttgggcagaaaggggaggaggttgtagggtaaagggagaaaccaagggtttagacactggtgggttaccaaggaccctctcatccagtgccatgccccatactgatagttggggaaggtaggaattatagaactctaactaggttggagattttttcttccctccacccaaggatgttcccaggaacttatatttaacataagaaaagagagatctcaaactgaagaatattacgtttcaatgacccctaaccttcagagttgaggctctaagtagaaatggacttacttcaaaatttcttgcactcttgaaattatactatttacagaggcactcaattaagtccagctctttcaatgttcattttagcctccaaagatatcatcctgaggtatcatgttgtacttgcagggccctaagcagccacctggtttgcctcatggtttaaacaactgataacagttccataacgttcttcttactattttggaattctggctgtactttacggtatttctttcctttaagaggacttatctataaatgtaatggcagcaaacatattggagactaacaacttccttacaagagacactaatatgaagcagagtgcatttgagagtcaaatctttggcacagtaagtaaaatttctctagatcccaggagacctcagacactctccagttatattaacattgcagaaaatttccagggccatgaatagagacatggcttatggaaactctgaaagatatctttcatgcagagtttgaaaaatatcattcagagtttccataagccatgtctcaatagacaaaactattctagggcagatgacatcccccaaacaaatattttcatgaattttacattttcagtgcattgcaagttttaaggattaatgttaaggttgggttttaaatggaagcatccctgaatgaggttgatacctcattcttttttttttttaatctcaatcgttgtaactttctctttcatgagtatctgataactgctttcacctagttgttgcataaatgcatttctagacatattgacatattttatgtctgcctttcaatcatgcttcactttttccagtaaatcagttactagaacatactgaagatgctgtacaaatgccttggtttctttctgccaactgaaacagcttgtttggggctcatttttcattacgttttatagcagggtgtagtgctccagggaaaaaaggatttttgtgtttatccctcagctattttctttatcctccattggatttctcagttcaactttcttaatattttaatttttctctgacacatacacatatatatatatatacatacatatacatatatacatatatacacatatatgcatatgtgtgtgtgtgtgtgtgtgtgtgtgtgtgtgtatatatatatatatgtatgtattattgggccttgtagaatctgcaataacctgcaatgaggaccaacttacagcttagcttataggtatttttttctaaaagtcaccattagcgagtaaaattatttcatcattcaggaagaattattgatctaaatatccagctgcaggggagaggggaattcagacaattgctatcagctaccatttctccaccccattaaaagagtatattccaaaattaagaatatattccaaaattaagaatatattccaaaattaaggctgggtatggtggctcactcctgtaatctcaacactttgggaggccaaggcagagagatgacttgtgcccaggagaccagcctgggcaatataatgagaacttatctctacagaaaaatttaaaaattatccaatcatggtagtgcatgcctgtagtcccagctacttgggaggctgaggcaggaggatcacttcagcccaggaggaggtggaggttgcagtgagctgtgatcgagccactgcactccacagtccagcctgggcaacagagtgggaccctatctagaaaaaaaataaaataaaaaatatatatatacacacacacacatataaataaataaatatatatacacacataaataaatatatatacacatatatataatatcacatttggactttctggagatttgagacagttgtcaaacataaagcagtatgggctgggcacggtggctcacacctgtaatcccagcactttgggaggccaaggtgggcggatcacttgaggtcaaaaattcaagaccagcctggccaacatgatgataccccatctttactaaaaatacaaaaaagtagccaggtgttgtagtgcatgactgtaatcccagttacttgggaggctgaggcagaagaatcgcttgaacccgggaggcggaggttgcagtgaactgagatcgagccaccgcactccagcctgggcaatagagcgagactccatctcaaaaaaagcagtgtgtgtttcagttttaatgtatttcagagacagtatttgattatgtacggccacgttttatataaagaacactttgttttcctagagtctagaagacagcttggaacataataggtgttccatacatttctgctaaataaaatagttgttttaaaagcacaccacattttattattgttacccatccattttaggttaaagaatttgacaccaattttacatatgtgcaacagtcagaattctacttggagccaaacattaagtacgtatttcaagtgagatgtcaagaaacaggcaaaaggtactggcagccttggagttcactgttttttcataaaacacctgaaacaggtgagtgtacttatatattttattctgttgggcttttctttatatatcttttctgctgagcacagtggctcacacctataattccagcactttgagaggccaaggcaggaagattgcttgagcctaggagtttgagactggcctgggcaacatagtgagaccctagtctgtacagaaaaataataattattattagcctgggtggtagaatgcatttgtagtcgcagctacttgggaagctgaggtagtaggattgcgtgagcccgggagtttgatgctgcagtgagctatgatcatcccactgctctctagcctggaggaaagaccaagaccctgtttcctaaaaagtttaaaacagccaggtgcagtggcttatgtctgtaatcccagcactttgggaggccaaggtgggtggattaccttaggtcaggacttcaagacctcctcggccgacatggtgaaaccctgtctctactaaaaatacgaaaattagctgggcatggtggcaggtgcctgtaatctcagctactcggaaggctgaggcaggaaaattgcttgaacccaagaagtggaggttgcagtgaactgagattgtaccaccgcactccagcctggccaagagagagagacttggtctcaaaaaaaaataaaaataaaaataataataataaataagttaaaaacaaaataaagctacaagatattttttttctctttacctttgaccaaaattgacaaaactattctagggcagatgataacatttaaattgtcattttgctttgattttcttaaggcttactacaatttatataatggttctcaaacattcctgggtataaaaaccatctgggaggctgggtacgctggtttacacctgtaatcccagcactttgggagaccaaggcagaaagattgcttgagcccaggagttcaagaccagactggacaagatagggagaccccatctctaccaaaaatacaaataaaattacccaggcatggtggcccacgcccatagtcccagctacttggggactacaggaagaattattgatctaaacaggcttcaatcatagctcactgaagcctctgcctcctgggctcaagcaatcctcctgcctcagcctcccaagtggctcagactacagatgcggtccaccatgctagctgaggcaggaggatcacttgaacctgggaggtcgagaatgcagtgagccacgttcatgccactgaactctagcctgggtgacatcctgggcaattgtgagccatacaataagctaagaacttgttttgaaaaatattatctgggtgtggtggctcaagcctgtaatcccagcactttcagaggcggaggcagttggatcacttgagcccaggagcttgagaccagcctggccaacgtggcaaaatcccatctctactgaaaatacaaaaattagctgggcatggcggtacatgcctgtaatcccagctattcaggagtctgaggcaggaaaatggcttgaacccaggagacagaggttggagtgagccgagatcgcgccactgcactccagcctgggtgacagagtgagactccatctcagaaaattttattaagaacatattctcctaaaagagacacaaaacaatcatatcaataaacgttatctactatatgtaaagcaatcagaaagttaaaactcctaaacacagaattctcttctcctcaacaggatttagctgatttattgtttatctttagatattttcctgcttctaatattattagtttaattttatattcatttattcatgccattcaataacatcttagccaaatagacttcagttgaagcacataataaaatgaatctattgtaattgtaataatactcctaatggccccattaacctaaagatgtctccctactgtaggtaccttatacactagatttaacattttcttttcagaactgaaggactttattataatcccagtatgaatagcatcattattataacattctggaattgtgttttatgttttcaaggcactttgacatacattataaatattgatggctgccaaattagtagagggagcaaagagcatgacttgctgcaaagctcttctggtatctcctactgcccaaatcttagaaatactttggccaggtgcagtggctcatgcctgtaatcccagcacattgggaaatggaggcaggtggattgcttgagcctaggagttcaaacccagcctgggcaatatagtgagactctgcttctatttaaaaaaaataaaaataaatagaaattaaatttaaaaatgttaaaatgaagaaacaacttattagggaatccttttctctttctttctttctttcttttttttttttttttttttttttttttgacagtcttgctctcttgctcttttgcccagcctgaggtacagtggtgtgatctcggctcactgcaacctccacctcctgggttcaagagattcttgtgcctcagcctaccgagtagctgggattacaggcacccaccactatgcctggctaatttttgtatttttaatagaggcggggtttcaccatggtgggcaggctggtctcgaactcacgacctcaggtgatccgcccgctttggcctcccaaagtgctgggattacaggcgtgagccatggcacccagccagggaattattttcagtatcagttctcttttttaaaagttagtttttaattgaaggtattctgtcaagttgatactgttaacaattcgtggactttagatgccattttataatagtcagaatttagttgataacccttttttttaatgaactctataactgcctagcaagattatgcaaattgataactaccatttatcatttacgaagtactcctgtgtataagcttgtttgattatgatgtcagccatatttggtagtgtaattagcgctactttacaaaagcggaaactgggcatgacttactaaatagtacattgctggtgggtaatgacacctaaactataacaaaacttttcttattcaaaatattgaactgcttggctaggtcagttggtagagtatgagacttttaatcccagtgtgaaaatactgtgcattttccccaccatccctcagcaatttcattctttaatttcagggaagcagaggagcaacttacttaagtattctaagtataggactacaaatgttcttctttaaacataaaagtcttggcgaggtgtggtggctcatgcctgtaaccccagcactttaagaggccaaggcgagtggatcacctaaggtcaggagtttaagaccaccctggccaacatggtgaaaccccgtctctactaaaaatacaaaaattaactgggtgtggtggcaggtgcctgtaatcccagctactagggaggctgaggcaggagaatctcttgaacttgagaggcggaggttgcagtgagccaagatcctgccactgcactccagcctgggtgacagagcgagactctgtctctaaataaataaataaataaagtaaaataaagataaaagtcttaagcttcaggtagaaggaaataggaacaccacagtttaaatttaaggtctgtttcctgaggagaaaaatcacttaagagacaaaaataccaattaaaattaagtatccctgaaaacttggatttattaaagtttaacatgttagctaagagaaaccatagactgttctcttggtacaaattcccttctaagacacattacatgagaaacagtaaaagtgtgttagggaaagtgctcatgttaaatctctttgaaaatgtacctttttttctgtgtgtaaaagcaatgtaagtttactgtagtatgcaacctaaaaacatccactatttacatttatttaatttaatactagtttttcctatgcatttttttaatgtttggggctatgatgtatatactattttatatcctgattttcttacttaatctaccctgttaagtttttaaaaactgatttcatggctgcacggcattctcgtttatgtatgtactataatttatttaggcatttccctacctaaataacatctaggtcatttccattttatccattatcaataaacttctttgcatagctttgtatataaatggtctttattcctttagttctaaagaagaattattgcatcaagagttaagcaccttttaagatgctgatgtatgttgtcgaactgctttttaccgaatctttaatattgattgctttttaaaaagggacctatgaaaagacagtcattcacaaaatactcatttagcctctaccatgtgccaggcatttttagatccttgcagaacctcagtgagcaaaggagacaaaattccctgccttggacaagtttccttctggagaagacacacagtaaaaatcaagcataataattacataaattacattgcatcatacataaagatgtaaataaaagcagtaagaatcagacttcacagctgaagtatgaattgcctcacaaatgaaaaaataaaatgatatcatttctgtaattatttatttaaccaacttgtttgataatgaggttttttggccagccaaacttttccagtgcttaaaggtttatttatgaagaaacacagcccaggcagtaatgcctcttccaagcaggacttttctgaattagtctctaagattctgacatgtgtattactgaattgattcatctcaatacaatgtgtttgcaaaattctgcccagctaagaccagaggacaaatcctgaagtgggtatggcttgtcctgcagcctcggcattcttattcttttctgttgctttccctctgaatagtgcctggcttggccattaaaaacctggtctcaaaaatgaaccaaaccaactcacttataatcaactttaatgacttattctctttttcttacttagagtatttagactttaataggacaaacaaagccttatactgaaaaagaaaatcaatcctccagaatacccatttattctatctgatataatagcaagaggccaattaacagactgaatcccagggtgacttcaggataataaataataatagctaacatttattggatgcttactaagtgttgggcattttgataagcattttacatatgaattgttctttatttatcactgcagctctataaaaggagtgtaattattatcccagttgcacagatgaaaccatatggcccaagcagttttgtcaaacattacaaggctggtaaacagtagagccagggtctaactcaggctatgggattccagcacccatgttatttatagctctgctagtctccttcctaagagacctaggagggtcctgcatccgcctgtgaaacaaaatcattctctacacattagctagtaataaaactatgccaacagagcttcatttctaatgcagtgtttgttatggaaatgcaaacgcatgagaaaagaaccagaagatactactgactactgctactttggcttgaatacaagatgtggaagcagatttgcagtagatgatgagaatgaaaccatagtactataggaggtttctgataggataggtaaggaaggggcagtctgagatgaggataaaggataggtaaaaaaggcagccttagccaagacaggaccatcatgggcaattcatcatgtgctttaccagctaattatgggctatctagaaagcactgtttctcatagtagggtctgtggacaatctacattaagaatgcttgggcactttttttttttttgatacagtctcgctctgtcacccaggctagagtgtagtgcaccatctcagatcactgcaacctccgcctcctggttcaaacaattctcctgcctcagcctctcaagtagctgggactacagacatccaccacacctggctatttttttttgcatttttagtagagacagggtttcgtcatgttggccaggctggtctcgaactcctgacctcaaatgatccccctgccttggcctcccagagtgctgggattacaagcacgagccactgtgcctggcctgaatgcttgggcacttcttaaagtacagattcctgagccctttgccaagcgatacttctgcagtgaagcataaaatccattgctgtagaggtcagacacactctttaagagaaggaagtgtcatcataaaagacaacatagggaatggacagaaaatgtggacagaaaggcagagtggatatgattgcccaagccattgaaacgggagagttccctgactcctgtcgcatatcatgtggctcatctattctgccaaggcacatgctcaaacccgttacaggagggggagcacacagatggacaggtgcgcaggagctggggtgagcaccttggggctccagccccacagtagcatctaggggtggatgcctgtgactcccaaagcccaagtgggaatgtgatacagttcactattttagctttgctgtctgcagacagcttaactgttaaccagctcagtgccctcttggtacccaggtccttgtccagtgtccaggaagaatcaggtcacacacagatttgaaggatgaatgtgggggttttattgagtggtggaggtggctcttagcgggatagatagggagctggaagggggatagagtgggaggatgatcttcccctggagttggctgtccagcggccgatctcctctctgatcgtccccaggggaacttctctcagcattcagatgctccttttcttctgtccttctctgccgagccattctgccattcttctgctcttctatttatctctctgtctgcttctggaacctggggtctggagtttatgagggtacaggatagcggggcatagcaggccaaaaggcaacttttgagcacgaaaacaagaatgcctgcttctatttagggctatgggtttccaagcttgagggtgggacctctgccagggaaccaccctcatttattcagtattttcctgtttgctgtttgtatcaccatcgccttagtagtagtggtttgggtgacagaaaagggtgggcatgtgagaatcttaggccattatctctagtactatctggtcacctctctgtttcctatttctcctgagtcactgaccaccccagcccacctgggcattacaggaaccccccaaagcacaggatataggactgttgtcgtaattaaccatcacatcacacatgaccctcttctgcctagaactcaccctgtctctggatgtgaggtttcagctgagattatgagaaccgctgagagctgctgaagttagagcttctgatccaccaaccttgataggcactgttatcctggggtctttcagcagtgcggaaagagccccaaaggggaaaagacaatttgtcattgtagaaaaccaacactgaaaatgtataagtcagtagtctaaatttgctactactgacttaaaaaagctgaaacaacaatggctagccaggaatctttttcaattctgcattgtaatatagatccctttttaacgaagaaaagacacaggttctataattcctttaataacaggttaaaggcttaagggaagggcagtcacatttcatgatgactaattcacttgcttgaattgctgcttgccatagaaaagtttcccagagaactcatattactctttttttttttttgacagttttccagaggaaggcaagtatgagcaagatattaaacaacattttgctttcctctcacaaaacagtattggttgttgcactaccagtgactccttgaagatgctcattaatttttcaggtttttaagtggctaaaagggaaaaagcaaatattatgttaacggaactaaatatcagtgctattaaatataatgcaaattagatgatggcacacagaatgtatttttcaaacattttattatgtgtaattagagatttgtgaattgtccacatagagatgggttctgaacttgtagactctattggttactaacccatgttaaacagacacggtgtttcttgaaattctaatcactttagacttttcatttgggtttaactttcttcaggtcctgcctctgataaaccaccaagtttctgcagaataggaaacaattcagaagagttccataatctccctgaagtttgcaacctattactacagaacaaagatctttctttctttctttctttctttctttctttctttctttctttctttctttctttctttctttctttctttctttcttcctttctttcttttctttctttctctttctttctttctttctctgtctctctctttctttcttttctttctttctttctttctttctctctctttctttctttctttccttctttctttctgtctttctttttccttccttccttccttccttccttccttccttccttccttccttcctcccttcttttttagacagggtctctctctgtcacccaggctggagtgcagtggtgtgatctcagctcataagatctgggctcaaacaatcctctcaccttggcctttcaagtacctggaactacaggcatgcaccaccatacccagctaattttttttcttcctttttttttttttttgtagagacagggtttcaccatgtttgccaagttggtcttgaactcctggactcaagaactctgcccaccttggcctcccaaagtgctgggcttacaggcaggagccaccatgcctggcctatgattatgctttttcttgaagtcatcatcttctatattagtttcctattactactgtcacaaatcatcacaaacttgaaagcttaaaacaacatgaatttattatcttatagttctggaggtgagaaatccaaaatcacaaccactgggctgaaatcagggtgtcaatagggttatgttccttctggaggctacaggacagaatcagtttcattgctttttctatattctagaggctgcctacattcctcagctcatggctccatccttcattttcaaagccagaagcatagcatcttccaatctctttctctgactctcaaccttctcccttctgcttataagaactcttgtgattttaccagatccacccatgtaatctaggatagccacttcctctcaattcccttttgccttataaggtaactgagtagaaagaacatactcaatttctcttgctatatagtactttcacaacacaggtcacttctgtgacctctgattaccaacatgtgtatggggatttctccccctcagcaacaaattatctggcagattctttgagggacaccagctgtgtgtcctccaagtcaattcattctggcactatctgcctagacatagtgtcagatcctacaactcccacctcagatgccagtctcaagtaataggtggttacctatacttccatctgattggctataaattagagtttgtttcctgatctttttttcagatttgatcaatttgctaggatgccccacagaacttgggaaaacacttatgattactggtttatgataaacagcattacaaaggatacagtcgaacagccagatggaagagatgcagtgggcaaggcatgtggggaggggcatggagcttccataagctttccaggtgcatcacctgccaggaacctccatgtgttcagctatcctgaagctcctagaacttaggcctttggggttttttgtaggcttcattacataagcatgattgattaaaccattggccattggtgatcaacttaatcttcatcccctctcccctctgggaggttggggatgggttgaaaaatcccaaccttctaatcgtgccttgatctttcctctgaacaacccccatcctgaaactacataggggcggccagccactagtcagctcgttagcatacaaaatgaccgttaccactttgggcattctaaggattttaggacttgtatgccagaaaaccaggatgaagacacaatatgtacttcgtaatattatagtaacctgttcacagattccagggattaggacacggacatctttatgatgccattatttgtcctactacatctccctacctccccaaccactaccaccaattgcagtttttactagtcaggatgattaggtgtaagcaaaataaatcaaggctggttatcttaagcagaaagagaattattagaaaggtagctgaaaatcagtagaaagcctggcaaaatgggcatgaacaggatccgagtgagagcaggacccagaaccacagccaaaatcacagcctaggaaaagtctggcgaggaccctgcagctgacgctgatcactgccactgcctgccctggtgccactgtcactgtaccctggatacagctatggtgcccacattggattctaagtggtttctggaagccctatgtcaacccttcaacattcacagcctgggtgttaggggagggggcatctggttggtgaggtttaggccatttgcttgtagccctactgcctctcagcttttttaggatgcccacaaagaagtatatcctagtgctgggcaaataaaacatggcaaatgtctgctagactatacagattggaagatgctatgcctggagagcaagggtgtacttctgaggaatctgttggccactgtgtagacttggaagctgtccattctttcatatagagttttgctacactttgacatcagaccttctctgtatggtccttgtcatttaggaaaatttcctgggtggctactgctttttgccttttcctcctcttattctttctttttaacaatgtaactgccatatgtagatttcttaccttctccctacataccaagtacctaaagcccaggaatataagaaggaagataaacccttgaatatgtctgtgtgtaccctgctacccttatatacaccgctgatactataatccactaaatgattcattgcttgacttgtttttcaaggtgtggtaatgagtttgttctgacacaccgttttccctgccctgggcttctggtgcctatttgatatgttggcattagttttgccttgctaattgacatactcaactctttacccttggcccgataatgttgaattctgaccacagagctaaactggcctggcttggagaaatggcacttaagtgctgtgaaacagctgaattattaaaaggagatgcgaaagagagaaaccaaaaggaggtatttgtgttttgagaaaaataggagggagggtgggggcgaatctccaatgatttaaaaaaaaacacatcctttgtctcctttaatatgactcctctttcttgaatatctagaacccctgaaaggtccagaaaagtccatgtgtctgctctaatcaaagcctttatgatgcaggccttggttactgtggctttgacatcaagattggcaagcgcaaaagtaaataaagaaaccttagagatgtctataccctttggctcagtaattcctcttgtagagatctggccttaagaaataatcagtggaaggaaatttatgtacaatgatatacatccaaaacctaattataatagtaagaatttggaaccagtctaaaagttcaacaatagcaataattagtgcttattaaggttttatttgtgtaaaacatggtactaagtgcttgatatgaactgtctcatcaaacctcatgacctcactgcttttatctaattttacaaatgaagaaactgagtcactgaggggttaagcaatttgcccagggccatggaactccatctagtagagccaggacttggactgagtttcgtttgagtctgcagttggtgtttgaaaccacctagctatacagtttctcaataggggtaagattgaataaatacagtaaaacatgatgaaacattaactagccatagcatgatgttttgaacaatttttaataaaatgaggaaatttttattaagtaatattgaataaggctgggtgtggtggctcacacctgtaattctagcactttgggaggctgaggcaggcagatcacctgaggtcaggagtttcagaccagcctagccaacacagcaagacctcatctctactaaaagtacaaaaattagctgggcgtggtagtgtgcacctgtaattccagctactcaggaggctgaggctggaggatagcttgagcccagggggcagaggttacagtgagccaagatcacaccactgcactctagcttgggtgatggtgtgagactctgtctcaaaagaaaaaaattgaataaaaaaggcaagacaaaacatttataaagagtatgatctccactaggctaccaacatacacaaaagactgttaacattttttttaaataatagtttctagtttgcaaacaaatataaaattgtattatcttctccatacccttctatacttttcaagcatcctgtaattagcatgtattacttgtgtaataaaggaaagtagtcggttttaagaagaaaataactctacgctagaagatttaaagcatcagtgctaaatgggttagcacagtagggcttgtggggtccagagccccacaaggccaggcagccccaacacaccatggcgagtcctcagtggagcaatgctaagtctcatcctgggagaggctgcttgcagggctgactctgcctcccaccctgagccatcctgctgtggaagtgaggttattaatttaatgagatcagtgaccagcacaaactacatgtcagaactggatgggaatttccactcttcaccctctacctaggtgactttgagcaagttatttatcctttctactcttttgtttccttatccgtataatgaggataataatattgtctgtgtgattgggttgttgtgaggattaaatgagagaaaacatgtaaagtttagcatgttacctgtgacataattaatactcattaaatggtcactttaaaaggatgacaaaacacactttgccccatggttgatgatatggtttggctgtgtctccacccaaatctcatcttgaattgtagttcccataatccccatgagtcgcgggaggtaattgaatggtgggggcagttgcagtcatgctgttcttgtgatagtaagttctcatgagatatgatggttttataaggggcttttccccctttgcttggcacttatctggcctgttgtcatgtaagacatgcctgtttccccgatcaccatgattataagtttcctgaggcctccccagacatgtggaactgtgagtcaattaaaattctcttctttataaatttcccagttttgtccaggtgcggtgcctcacacctgtaatcccagcattttgggaggctgaagcaggtggatcacctgaggtcaggagttcgagaccagtctgaccaatatggtgaaatcctgtctctactaaaaattccaaaaaaaaaaaaaaaaaaaaaaaagccacgcgtggtggcatgctcctgtaatcccagctacttgggaggttgagacaggagaattgctagaacccaggaggcagaagttgcagtgagccaggatcatgccactgcactccagcctgggcaacagagggagattctgtcttaaaaaaaaaaatccggttttgattatgtcttcatagcagtgtgaaaacagactagtacggttgatgtagaaagaagagctgaggtgatgatttggcatcatccttaaaatacagatggaatacgttattgctaaaaccaggtccttttgagtggatttgattaaactagcctggtgttttggtaggccaaaaaatatagttgttatgctttaaattttgtccaacaataagaaaccatatttctcgtttgagatcactctaaattcccacaggcacattgtcttcttgtaagactaaagtttggtgccagtgtgtacaagttatataaaaattcttcccaaattaaagataatttggattttttttagtatattcaagtatgtcctgtgagattaataggcataagttaatattctgttgcaaggatggaactgtctcattatggacgtaacaccacaaacaccaaatacagtaagatatcatgaggttttttttttttttagcaaattaggcaatgcactgatgacagaactgatataaaaaacagtttccagtcataaaggctgtgtgtgtgtgtgtgtgtgtgtgtgtgtgtgtgtgtgtgtgtgagattctgtgtttgctaatgcttttgtatgttaaaatacttgcaaataccataaagcactaatagcaggtggattaaaaggatgggattagggatggttctttttttctccttttaatttctcctgttgaaaatttttctgtactttaattatcgcttttacagtcaaagatgttaagtataatttaaaaagaaatgacgtagccacgtggtggcagtattccatgaagaatgtaatctacatagctttgtttcagtcctatgtctaacattggcttgatttcttctctgacaattttgtttataaattgtcttattttagataactgattccttgtgaaatttccctatgaggattaagcacctcttataagtgactttaatttaatgcacctgtttgatttggaactatgataacaaatagactctaaaggaagattgtatttattccctgttagggctaataaaaccctagtcagatatgaaaattgtttttatatgatgcaaattaataactgaatgaagtttcattgccaataactacctgtagtttatacaacagaattacattaacctgtattaaaagccaacaacaccatatattacttttaaattaaaaatgctaattttaaaataataaaagtgaaaacaaaaatactacagcagcatatgctataagcccatgattttgctaaattgaaaattgtgttaccttttaatgtagtatttttactcttttttttttttttttttgagatggagtcttgttctgttgcccaggctggagtgcagtggtgtgatctcagctcactgcagcctctgcctcctgggttcaagtgattctcctgcctcagcctcccgagtagctgggactataggcacctgccaccatgcctggctaatttttgtatttttagtagagatggggtttcaccatattggccaggctgatcttgaactcctgacaccctcctcagcatcccaaagtgctgggattacaggcatgagccactgcaactggccagtatttttattcttaaaatttcaaaataatggagattcagactcctgcctgagctccctgcaaatgaacattggcctgtaaagatagcactttagatgaataaacctgactgttggccatcagggcataaccttgttccctcagagtttggtggaaccatatacccatggagcaaaagagacttagcgaggcattgagaagccaaaaacaatttctgatttgtcacaattccgggtgaaacattttcctcttgctgtagagaagccaggacaaaaaacacgggaaataacttttggcttttctgatgttttcatcttctaaaatccctcacccactgttctcagaaaactaccctcctcacgcttatgcctgcacccagagcagccttttttcttttttctttttttttttttgtgaaatggagtctccctctgtcacccaggccggagtgctatggcacgatctagactcactgcagcctccacctcccgggttcaagtgattctcctgtctcagcctcccaagtagctggtattacaggcacgcaccaccatacctggctaatttttgtatttttagtagaaacagggtttcaccatgttgcccaggctggtctcaactcctggcctcaagtgatccacctgccttggcttcccaaagtgctgagattacaggcatgagccactgtgtctggccagaccagcctttttcaaaaatttctgaagccccgtagaaaccttttcattggaaatgtaggacacttttctcaaagacagtcacatgggccacactactccttatacaagcctgtgatatctcagcctcaagttgtgatttctccctgcccccaaaagtaatttttttaaaagaaaaatttgagcactgaaatcagagtgacggaatacacagggcttgaagcaatctcagggatccaaggcttagagccatcttcagaaatgtcttctgccagttgggagaattgtgtccttgaagtcacttctgtcagcttttaattatcaggaaggaggagactggcaaggctgcaccaggacccctttgagttcagactgaaagttaggtaccagggttgctcaccccaccctggtcagaatcattcattagcagtttcctgacagcctttataactagaccaggctgccaggaaaagaaaagagcagagagaagtcattggtgacatcccacccaggtgttacaacaccaaagtgcctgctaaccaaaattaggtttcttatgaggagccaggagaaggtgaacagcatcccaaagattgtgcaaaggcaaaagtgacacatcttggggaccctcaaggaaatctgaaccttattgcaccatcaattgcaaggaatcaaatagagattagttatcatgcgttttttctggctcagtaaaataaagctctttggtatttgctgcctagaagcctcatgaaattagtggacacaccttgaagttttacaacctaggtagattatagttgcccttgcagtgtttcctcctttgagatttacccaattaaataaaaagaagaagaaaagacacatactcgaagactattgcttttattccacccttcaagcccattaaagttattggtttactttagtcatctctaaacaagggaagaaactccgttgggaaatttggcaagcagagtgcaaaatgtaagaaactgacactctttccttttggtttaagttccccaggtcacatcaaaagcattccaacatgacacatggaattctgggctaacagttgcttccatctctacagggcaccttacttctggtaagaaaatacaacttaggctttttgagtagtcttttagtaattgcccattttaacccatcatactgaaaaaatcacatcaggtgttaagtttctggacaataagatatgccttatgtcttccataggaaaataatagacaaagtacaaagatctgcttaaaactgaatgtaagaagtggcttaggtggattttgccggcttttgcaatagattgtatacattttttaaaatttttatttattttattttattttttgagacgaagccttgttctgtcacccaggctggagtgcaatggtgcaatctcggctcactgcaacctccgcctcccaggttcaagcgattctgctgcctcagccttctgagtagctgggattacaggcatccgccatcacgcccagctaatttttgcatttttgtagagacgggtttcaccatgttagccagactggtcttgaactcctgacctcaggtgatccacccacctcagcctcccaaagtgctgggattacaggtgtgagccaccgcacctggccacatttttattttaatagctgctagggctcaagacccaaaaacttctttcaaaacaaattacttacctatcttgtgctaggacttgtctagacatcttcttcaatctttaaaacaacccatgagataagtgttacgcatctattttataatgaggaaactgaaacttagagtagttgaggaaacttttcaaggtcatagagctgctaagtgacagactaaaattcaaatccttttctttcaatgtcctggagtctattgtctttcttttatacaaaccagctcccatttcagttgttagcatgactattatcgtattgatgagcttgcctaaacgttttaggcgtaaaaaaattgagatctggttgtacaatgggtgaacataactctaaatagaaatttcgagttgaaaacttttaggcatgactatttcaccattgaccactgacagaccatttatctccttgtgtagaaatcaggcaggaatagtataagaacttttgcaggttgtattgctattctttgcaccttcatttatttaatattcattaagcaactcctgtgtacgaggcactgttctgggtactggggaatcagcagtgaaaacaatgagcaaagcccctgtcttcatggagcttctattctagccagacagggcagaaaaacagcaaacaaaacaagaagaaaagtcaggtggtggtgaagtgtcgtaaagaaacatgaagtgggtaggcatggtggctcacattttgtaatcccagcactttgggaggccaaggcaggcagattgcttgagtccaggagtttgagaccagcctgcacaacatggcaaaaccccatttttacaaaaaatacaaaaattagatgggtgtggtgtcatgtacctgtagtcccagctacttgggaggctgaggtgggaggatcaactgagcaccagagatagaggctgcagtgagctccactgcattcccgcctgggcaacagagcaagaccctgtctcaggaaaaaaaaaaaaaagaggaaaaagaagaaaaagaaaaagaaacatgaagaaaggtaagggcactctgaattatcaatcaattgcaagccaagtgcttaggttcagtacagttccctaattatagatgcctacacagacctacctacaccttgatatttctgtgggatcagtggaggttaggaactcatggcagctctgttagataagtattattatctctattttccaaaagagaaaacctgagactcagcaagttcataattatgccccaaggtcacagagctgataagaggcagagtttaattcaaacccaggtatatcaggccacgctcttggtcattctgctctactgcttagacccctttgccgagcactgtgttgacctgagggctgtctatcctcttccaggcactaattaatgcccaagggtgtgtggcagccagtacccccaattagttgcttcaaatagtcagaaatagctctttagccctgggaaaacattaatttcatatcccatcaagattttgcagtgagctagagttactcagactcctgcctgctgattggctttagaagagggctgggcagcctggagaccccggtatgtgaaataactagggtggggcaaacacagtgtctgccacatggtaagtacccaataaacatttgctgaaggaagggagggagtgaggaaggaggatggaagggaagccaatacccaacatggctctagaacaaattccaatgtgataagtaatgcctcaactatcttctatatttgaaaatagggctttttcatgtaccagggagaaagcatgatgagcctggtgggtaatatgtgttgaataaattatattaattatttaaatattttaggagattaactcaactttgacatgcaagaaaagcattggttttgtttgtttgtttgtttgtttttgagacacagtctggctctgtcgcccaggctggagtgcagcggcacgacctcagctcactgcaacctccacctcccaggctcaagccatcttcctaccttggcctcccaagtagcggggactacaggcacatgccaccacacccggctaatttttgtaatttttgtagagatggagttctcactttgttgcccaggttggtctcaaacttctgagctcaagtgatcctcccacctcggcctctcaaagtgctgggattataggcgtgagtcactgtgcccagccagcattgttttttaactaagtgtgagtgcagcataaaggcaattttgatttcttttttattttttatttttatcttaacttgccagtgtatgatttctttcttatcacgtatattgctctaagtaaagtggatgtcctttctcagcaggaaaaatttcatgagcaatagattttgaagaatgatgacaaattgcacacattctatctcacacaaaaagttaaaaaaaatcagtatgattgtaaccagctttagacattgttacagcaattgggaattctcacctgtgtcagacaagccaaatgaagctcaccactaagaatttatacgaaatttgcatgcacaagccgaccacatttgccagagatgcacttctaaaaacccactgacatcagatacatgtagcccaactttctcaaacaaaaagttgtttcctggggtagttgtgcactctggaaaaacagtcactctgtggcctaaagtaaaggttaattttgcttccccccaccctttctcctttgagacctttgctttgagcagagtaaagagaatagtaattctggtatcaaatgaagactaatgcttggttaaaattatttttctttcctttcattagacaacagaggagacattggacttttattgggaatgatcgtctttgctgttatgttgtcaattctttctttgattgggatatttaacagatcattccgaactgggtaggtttttgcagaatttctgttttctgatttagactacatgtatatgtatcaccaaaatttagtcatttcagttgtttactagaaaaatctgttaacatttttattcagataaaggaaaataaaaagaacaatgtttaataagtacttacccatgccaaactctctacaaatgtctttcctttaatcctcaaaatgaccctgccagaaaagcttcctggcctattttacaggtgacttaaatgaggcttaaagaggctaagtcctcagcccagaatcactgaacagtaagccctgagtccaaacacagctgatatcaaagcccatttctctcctttactacacggcgttttccattgtgcctcaaatttacctacagtgcctagattcttgagagtggtgaagtcacaaattgccttgtgttaaaagaaaaacttcagccaaattaaatttaaaggagtttaattgagcaatgaatgtgcgaattgggcagcccccagaattacagcagattcagaaagactccagggttgccttgtggtcagagcaaatgtatagacaaaaaaaaaagtgacacatagaaattggcagtgaggtacagaaacagctggataggttatggattggcatttgccttatttgaacacagtttgaacacttagcagtctatgagtggttggagtatggctgctgggattggccaagactcagctattgttacgggcacatactactaagttaggttttcaatcttgtctgcctattaggctaggttacagttcatccacaaggacttgaatatagaagtatggagtccttctcagatcatatttagtttgctttaataattccccccttttggtgattttctcaattttgagagattgaccaaaactttagtcattgatgtcactatcaccattgtaaatgtacttatatggtctttaaacccactgggaaacagtagaacagtgggttctgtaaggtggcaacaaggactgagtagaaggtacctccttatgctggaacatcctgtttataggagaaaaacaaaacctggtctgttctaggatctatgtgtttccttaaagtcttagtttgactttgtcacatttagcatgaacaactccattttggtttggtttggtctgttggagtctagtgcatgagctcagtccaaaacaatggcctcccataatttcgttttaaaaaaaaaatccccctttttggtcaggttctcacttaggtgacagtgtgaccaaaacctagggcctcagtgccactctcagttaccatcattttgggtttctgttctcagcatgtaattcataggttatggtgtcctgatggtcacacatttcttttagctcttgttattccagttgaagagagaccatttgacattctggagatggctgcttgtaaacatttaaaacctttgagagaatacagcataccagggagattactattaggactattgggagaataatactaagagtttgggttatgttccttacccagggtcctcataaaccaaaccaaaccatctaaaaatcaaatagatcaaagaatgagctacatgaagagtctactcacttaagtgatttttttcattaatcccctacaactgaatcactataatacccgatgttttctccatacatcataagtgccagcagctgtttagccaatacttttcggtttggccaattctattatttcgcataactttcacaagagaatttaaagttttttgtgtaaccataggctttacagtagaatctgctatagaacctatcatgagggatacatttctaatcagtgcctcttttacttcaaactatggaaaaaagacctaacaaatgatgccctgctagaagagtgaaggcctcctggcaatgttctctttagcccatgatgtggcttaagaggaatgaatcaatgttctgtttctgactgactatgaggcaatatatgaacccttaaaatttctcacctgcattgggccttcatcttttatccatcaaagtataacattatccatgtataaggctggctgcaaaatccttcacaaataaacatataccccataatacacacataacagacccccttttcacttctattgttcatagaggcataagcaagaaaaaaaatattcaaagttaagagtctcatgatagtagagaagtcttaatccatgattttaggataagctgttcacattaaggacgctgtcttcttgggagaagctgtcctggttagctttaccttaagggttccgatgggtgtggagggaccctcctcagttgtgagattatgaaagttgtgggatctctgaaaccaaaagttcaaggtcccaaagttttgctgtagtgtggatggcaaggacagtctttctctaatgttctcagaagattcattctttgggttctagattgtgaaggggttgattgtcctcagtgaaccataaacagctttttttaactatgtaaaaatgtactgcagcataataatctactattataacatcagtcttcttgcatgggaaagctttcatacaaccagaaaacatgcattgaaagtgacaattgaatgaaatcccttataaatatttaaatgtccaatcaggtagccaaatgtacctgaagctttgattgttttcccaggaatatgggtttgacaagccaaatattgtttataactattttagtagtttataagtcaccacacaaacatatttaatttggatcattttatcttttccattacaagtcgtaaaatgcagaacttttaataatgaaagccttaaggactctggaaggataaggtggctgtcttggttctccacgagtccatgcttaaaaatggacttatgtcctcttgaataccagttgtttctccaatttaggtgcatagcactgataactgatgggttatcataggtaatttggcttagatcatggagtttattcaaattgtatatctaaacaattttagtattggctgatttagcatgcggatttctctttttttttcttatactgtaagttctgggatacatgtgcagaacatgcaggttcgttacataggtatacatgtgccatggtggtttgctgcacccatcaacccgtcatctacattaggaatttctcctaatgctatccctcccccagcctcctaccccttgacaggccccaatgtgtgatgttcccctccttgtgtccgtgtgttctcattgttcagctcccacttatgagtgagaacatgtggtgtttggttttctgttcttgtgttagtttgctgagaatgacggtttccagattcatccatgtccctgcaaaggacatgaactcatccttttttatggctgcatagtattcaatgatgtatgtgtgccacattttctttatccaatctatctttgatgtgcatttgggttggttccaaatctttgctattgtgaacagtgctgcaataaacatacatgtgcatgtgtctttatagtagaatgatttataatcctttgggtatatacccagcaatgggattgctgggtcaaatggtatttctgatcctagatccttgaggaattgccacactgtcttccacaatggttgaactcatttacactcccaccaacagtgtaaaagcattcctatttctccacagccttgccagcgtctcttgtttcctgactttttaatgatctccattctaactggcatgagatggtatctcattgtggttttgatttgcatttctctaatgactagtgatgacgagctttttttcatatgtttgttggccgcataaatgtcttcttttgagaagtgtctgttcatatccttcacccactttttgatgggttggtttgtttgttttcttgtaaatttgtttaagttccttgtagattctggatattagccctttgtcagatgggtagattgcaaaaattttctcccatactctaggttgcctgttcactttgatgatagtttcttttgctgtgcagaagctctttagtttaattagatcccatttgtcaattttggcttttgttgccattgtttttagtgttttcatcatgaactctttgcccatgcctatgtcctgaatggtattatctaggtattcttctaggttttttatggttattaggtcttatgtttaagtattgaatccatcttgagttaatttttgtataaggtgcaaggaagggatccagtttcagccttatgcatatggctagccagtttcccaacacaatttattaaatagggaatcctttccctattgctcgtttttgtcaggtttgtcaaagatcagatggttgtagatgtgtggtgttatttctgaggtctctgttctgttccattggtctatatatctgttttggtaccagtatcctgctgtttttgttactgtaaccttgtagtatagtttgaagtcagatagcgtgatgcctccaggtttgttctttttgcttagaattgtcttggctatgtgggctcttttttgattccatatgaaatttaaagtagttttttccaattctgtgaagaaagtcaatggtagcttgatggggatagcattgaatctataaattactttgggcaatatggccattttcatgatattgattcttcctacccatgagcatggaatgtttttccatttgtttgtgtcctctcttatttccttgagcagtggtttgtagttctttttgaagaggtctttcacatcccttgtaagttgtattcctagttattttattctctttgtagcaattgtgaatgggagttcactcatgatttggctcactgtttgcctgttattggcgtataggaatgcttctgatttttgcacattaattttgtatcctgagagtttgctgaagttgcttatcagtgtgaggagtttttgggctgagatgatggggttttctaaatatacagtcatgtcatctgcagagacaatttgacatcctcttttcctaattgaataccctttatttctttctcttgcctgattgccctggccagaacttccaatactatgttgaataggagtgcggagagacagcatccttgtcttgtgctggttttcaaagggaatgcttccagtttttgtccattcagtatgatattgtctgtgggtttgtcataaatagttcttattattttgagatacattccatcaatacctagttcattgagagtttttagcatgaagggctgttgaattttgtcaaaggccttttctgcatctattcaggttatcatgtggttttcatcattagttctgtttatgtgatggattacgtttattcatttgcctatgttgaaccagccttgcatcccaggaataaagccagcttgattgtggtggataagctttttgatgtgctgctggattccgtttgccagtattttattgaggattttcacatcgatgtttatcagggatattggcctaaaattttcttttttttgttgtgtctctgccaggttttggtatcagaatgatgctggcctcataaaatgagttagggagtattccctctttttctactgtttggaacagtttcagaaggaatggtaccagctcctctttgtacctctggtagaatgtggctgtgaatccgtctggtcctggactttttttgattggtaggctattaattactgcctcaatttcagaactggtttttggtctattcagggatttcacttcttcctggtttagtgttgggaaggtgtatgtgtccaggaatttatccatttcttctagattttctagtttatttgcacagaggtatttatagtattctctgatgatagtttatatttctgtgggattggtggtgatatctcctttatcattttttattgcatctatttgattcttctctcttttcttctttattagtctggctagcagtctatctattttgttgatcttttcaaaaaaccagctcctggattcattgatttttttgaagagtttttcatgtctctgtctccttcagtgcttctctgatcttagttacttcttgtcttctgctagcttttgaatttgtttgctcttgcttctctaattcttttaattgtgatgttagggtgtcaattttagatctttccagctttgtgatgtgggcatttagtgctataaatttccttctacacactgctataaatgtgtcccagagattctggtacattgtgtgttttttctcattggtttcaaagaacatctttatttctgccttcattttgttatttacccagtagtcactcaggagcagattgttcagtttccatgtagttgtgcagttttgagtgagtttcttaatcctgagttctaatttgatttcactgtggtctgagagacagtttgttatgatttccattcttttgcatttgctgaggagtgttttacttacaattatgtgatcaattttagagtatgtgtgatgtagtcctgagaagaatgtatattctgttgatttggggtggacagttctgtagatgtctataggtcccacttggtccagagctgagttcaagtcctggatatccttgttaattttctgtctcgctgatctgtctaatattgacagtggggtgttaaagtctcccactattattgtgtgggagtctaagtctctttgtaggtctctaagaactgactttataaatctggatgcttctgtattgggtgcatatatatttaggatagttagctcttcttgttgcatttatccttttatcattatgtaatgcccttctttgtctcttttgatctttgttggtttaaagtctgttttatcagagactaggattgcaacccctgcttttttttttctttccattttcttggtaaatcttccttcatccctttattttgtgtgtgtttttgcacatgagatgggtctcctgaatacagcactactgatgggtcttattctttatccaatttgccagtctgtgtcttttaattggggcatttagcccatttactttttttttttttttgagatggagtttcacttttgttgcccagactggagtgcaatggcacgatcttggctccctgcaacctctgcctcccaggtgcaagggattctcctgcctcagcctccctagtagctgggattacaggcatgtgccaccatgcctggctaattttgtatttttagtagagatggagtttctccatgttggtcaggtaggtctcaaactcctgacctcaggtgatccgcccaccttggcctcccaaagtgctgggattacaggcatgagccaccgtgcccagcctagtccatttacatttaaggttaatattgttatctgtgagtttgatcttgtcattatgatgttagctggttattttgcccattagttgatgcggtttcttcatagtgttaatggtctttacaatttggtatgtttttgcagtggctggtactggttcctttccatgtttagtgcttccttcaggagctcttgtaaggcaggcctggtggtgacagaatctctcagcatttgcttgtctggaaaggattttatttctccttcgcttatgaagcttagtttggctggatatgaaattctaggttgaaaattctttcctttaagaatgttgaacattgacccccactgtcttctggcttgtggggtttctgctgagacatctgcttttagtctgatgggcttccctttgtaggtaaccagacctttttctctggctgcccttaacattttttccttcatttcaacgtcggtgtatctgatgagttgtgtcttagggttcctgttctccaggaatatctttgtggtgttctctatatttcctgaatttaaagtttggcctgttttgctaggttggggaagttctcctggataatatcctgaaatgtgttttacaacttggttccattcttcttgtcacttttaggtacaccgttcaaacatagatttggtcttttcacatattcccatatttcttggaggctttgtttgattcttttcattcttttttctctgatcttgtcttcttgctttatttcattgagttaatcttcaatctctggtattctttcttccacttgattgactcagctattgatacttgtgtatgcttcacgaagttcttgtgctgtgtttttcagcaccatcaggtcatttatgttcttctctaccctggttattctagttagcaattcgtctttttttcaaggttcttagcttccttgcattgggttagaacatgctgctttaactcagaggagtttgttattacccaccttctaaagtctacttctgtcaattcatcaaactcattctccatccagttttgttcccttgctggcgaggagttgtgatcctttggaggggaagaggcattctggtttttggaaatttcagcttttttgtgctggtttctcccaatcaagccagtggatcttagcttgctgggctctgtgggggtgggacccactgagccaggcaccggagggaatctcctgatctgctggttgtgaagaccgtggggaaagcacagtatctgggccagagtatactgttcctccccgtacagtctgtcatggcttcttttgactaggaaagggaaatcccccagccccttgtgcttcctgggtgagacgatgccccaccctgcttcagccccccctccatgagctgtacccactatccaaccagtcccaatgggatgaacttggtacctcagatggaaatgcagaaataacccgccttctgcgttgatctctctgggagctgcagaccagacctgttcctattcagccaccttgccagctctccagcatgctgatttttcacgctgatttagcatgaaaatctggcatagtattttcttggtatttaattaatttttgttctactttggttagcagtcttataaaccagttagtctttttattaaagttttagggattcttaccgagtctaaatgatacgaatttaaagttattagaaacctgtattcaagagtgctttttagcatcctttttatcctttcatgaactttctaaaagataccatattctaggattttccgtgtttgtgaagttttcagaaattgcattagcattaagtaattaacttaatgtaatgactttaaacagtgatatttaaaaacacaattcacaaggaaatgtggttatctctgtggtctgtgataaattaacataataaccttaattatgattgaaaggatatactcagacattagaattttagaaatcctatacaattttggaacatatattaatattatttaacctgaagaagattaaacattgtttttattttgacaatcccatgtaactaaacatgtcagataatcctatttacctctcctttggatgctccaagggccctctgtagcatccaaaatttgggggttagaaagacaattttgaagctgaaatttcattttgggaagcctataaaatatgttaaaggtttaaaatacttgatataatgcttaaccagtttgaccatgaggtgaaattcttgtaaacattttgtaaccctttacaaatttttgttaaaaagcagatcagtgctctaagaaaaacatgttgtgcttttatttcaattttaatttatagaaaaacttagcaatacccctttatctttagccaatgtccacacagaatttctttttataagattaacttttcataaaccttccacaatgtattcaagcctttagctttattttaatctaatttaaaacaatattttaaccctctaacctagacaaaaatttacattcctgtgccttcttataaccttttagtaaaaacgcattttagtttcctgacacaccttgcatgtaaatctattttcagtagtctggattacatgttgtaatggtaactgttagcaacttttaattttggtgcctaaatttcctttcatgtatcctttcatgacttatgcagaccatctatgacatgcttagactttctgacttgtcctaatcatccctctttttaagcaaccagttattttactttaggacaagaatttaccatacaactttctttcttatataaaatctattttctttataatcttttttgcatagctaggggagcatggctaattccacatgtccccaggccttattgagaatctaatgcctccaagataggtaaattgaacaattttcaaaagtcaaagcagtttatgacctgaaagcattagtaaacctaatatctgacctgtctaatttagacaaaatgtctttattttaccaataatcgttaaagccatttttatttcccaaagattactaaagttacttgaactaagacattacagtttttattttttcttccaaaagtgttttatctaagcacttatttttctttaagccaattacttagagttcttttatatgaacatcacacacacaacacatatataactacacagagaacaagatccagtagttgtaggatttttcatttgccagtttcttcattggattactgaccttggggtggagcccatcaagaaatggctaggaaaacatgcagtttctggggtctaataagcaggcacagctggaaggcaaaatggatttcgagagagatctatttgcttttaattcttgaggttccatgaggaaaacagagggttttttccaaaacaggatcagtggcaccttctttatttttcccaaggagtcctaggctatcagaagttatcttagggcctctcatgtgtgccttaagagtggcaagacaaaatggagaaaaataattcagtcgactgagaagaaaaaacctttttcagcaaaacaagatccacaaaaaggaaagacataaatgtggccaggcgcagtggctcatgcctgtaattccagcactttgggaggccgagctaggcagatcacgaggtcagcagatcgagaccatcctggctaacacggtgaaaccccgtctctactaaaaatacaacaaattagccaggtgtggtggcaggcacctgtagtcccagctacttgggaggctgaggcaggagaatggtgtgaacccaggaggtggagcttgcagtgagctgagatcatgccactgcactccagcctgggtgacagagcgagactctgtctcaaaaaaaaaaaaaaaggaaaagacataaaggccttttaaatatacctatagcttggatatccacttttaattaagctgactcttaactatagtgctctttaaaaaatccttttaaatctcttgttacccaactttagccatgccaagtggcctgtatttctggcttttgaactttacctcccaggtgctcagagaaaggaaaattcaagacaattcatggaagggaagagaatcagcaaatgataaagttcccacaggtatcaaaccagaaaggactcattccttaacccaggaattgaacccaggccaccactgtgaaagtaaaaaactttagctactgagctacagtactgggtagtctccattgtgcttcccagaagggctctaaagtacttaattttgagcttgcaaaagcttttaactactcaacttaatttttagagctaactgtgacatgaaccctaaaattcctgttcccttgaaggcagagaccaagaaaaagtaccaccacgtggttacaaagtcaagctcccaagaatgtaaaacaagatggagacctcatccagtttttttttaatgcacttcagtgtgttgttgttcatttggaatgttccattgtaagttatctttagtaaaattttgctatttctgtaagactttgctgcctcccaagcctaatgtataaactggaaggaactctgcttttcagaaattaaggatctcatttttacctaaaatattggctttgctttcaggttcccttgatgaacttagccaatgatttttcctacctaagtgtgcaagaaaaatgaaacaaaggtgtagaaaaccaaaaatccctgtgaatttccaaatgccaaattttacaacccctgcaatactaccatttactaccagtttctttctgacctagtcaaatctaagaggcctctaattggatcctagcctgttaattactgatcaaatccaatcctggacccagtccagtttctgttgtgacttctgaacccagtttggaacaggaatttgctcaaagaaacttggagagttcaaaacacaaatctgtggagctctgaaatccaaaagagaacttgccatgatccccagccactctgagagatcaaaggacacaagtaggtccaacaggttccttgcttgttcactcagtgctcctgggggtcgttagaaggtctaattcatatcccgcttctgacaccatctagtaaaagaaaaacttcagccaatttaaaggagtttaattgagcaatgaacaatttgggaatttggcagcagaatcacagcagattcagagaaactctagggatgccttgtggtcagaacaaatttatagacaaaaaaaaaaaaaaagagagaagtgacatacagaaattggcagtgatgtacagaaacagctggttggttacaggttggcatttgtcttatttgaacacagtttgaacatttagcagtctatgagtggttgaagtatggtccactgggattggccaagactcagttactgttacaggcacatactcctaagtcaggttttcactcttgtctgcctgttaagttaggttacagttcatccacagggattcaaatatagaggtatgaagtccttctcaggccatatttagtttgctttaacacttgaattccacccaaacaaatcagcttgaatcagtgtgaagggaagtgtgacaatttttaaatttgtcaattccttaccaattgttttagtgttttagcattcagccccaaagtcccttactcttggcccccttcagttttttccctcctgtgatggtattagtaaagatctatggttagttatttaaaatgagactttgagaaaagcaagacccttggatttctaaattttactgatgcattgagtatttctaagctgctcgatagattagagttgtttggtgtggcagttccccagtgtgtccagttgctcacaaattttgacttgaatgttctttgccgaattggcactgagtttctccttcttgccatcatttgcttcatgaaataatctttctttcgtttacatttataatcaagtgcagtagaaagattttaaatgagctattataaagtctactaatgatttcttatctacataggttttttgctcagaacttaatatttcaaaatttaaattacacattaataaacatattcctaatacccttgtaaggaaggcaaactaatacggaattttatttgaggctgttttaaaatatacttgattatgaagtcccttgaaatattttaatgttcaaattaataccccataaaaaaatcaatatttgtgttattatttaaacatgccctagtgtaaaagtttaagtaacactaagttcataactaagaatagcctaaaaactaattttcagttcataacaaggaattagggcaattctttgctcatttaattatttaaataagtactctttttaaaaagataaaacaagacataattccaatatatttattttacatttgaacagatacttttatttaatacaactattcattatctataaaagccttagttctgaaacagaagtgagcaacagaaagcaaaatattttatttatttatttatttattattattttttgagacagagtctcgctctatcatccatactggagtgcagtggtgcaatctcggctcactgcaacctccatctcctgggttcaagtgattctcatgcctcagcctcccaagtagctaggaatacaggcacacaccaccatttccaactaatttttatatttttggtggagacgggatttcaccatgttggccaggctgctcttgagctcttggcctcaagtgatctgcctgtctttgcctcccaaagtgttgggattacaggtgtgagccaacatgcccggcccaaaacattttaaatataataaaaccaaaagtgggtaaatatatatacatatatatatatatattctcactgtatcagtgtactgtataaaaactttactccaatagcctgaatttactaatttggtctaattttaaatacagaagagagtgtgaattagtaaacaaaacaacaacaaaaaaaatggaatcataaaacattgtataaagatgagtaaagaaataatatttaccctaaagtccaaggtcaaattgggctagcaaagttcactcagccattttggttgttatttagtacaggtcctttgagaaccaatttaaacaaataagtctgatccgttgctaacaggatccattgttaatatgtaaaaaggtgtctgagatcagtttagtctcttctaagttcttggtacaacctttctgcccaaaccaatttgtactgtcaatgcagagagtgtttttaaacacaatggcagtgctaatagatagcttgggggaaaaagtgggatgtttatgacttctaccttttctttccaaagccccaccccactgatccttaaaataagcacatggtctaggtagaaaccagttctcactatgccacacaactgcaagacatgttccccagcctccttctccatccgcatcaagaaagtttggagctgtcaccatcattacaaccctatttgaaataattacaaaatctagactcagaacagcctaaattttagggctttattatataacattctctttttaaatatgcggtagttacggtcaccttggaaagttctacaaaatatcccttaagttttttgaactttcccacatgggaatcttctggttatgagagtttgctctatttaatatgtgtacggtttcactgctagggtggttctcccacttatcttgaatctagtgtgagtgttttgaaatgtttctgctcattgaaaagaagcagagcaatagagatgagaggaaaatctgaaaagataatgacacaatttcccacttaattttcattaagtaagagatgaaaactttagcctcggcatcaggaagtttgatttctttaattaattttttttttgagtcagggtctcactctgttgcccagagtgagtgcagtggcatggtcacagctcactacagccttgacctcccaggctcaagcgatctttccacctcagcctcccaagtagctgggaccacaggcatgcaccaccacacccagctaattttttaatattctgtagagacagggtcttgctatgttgcccaggctggtctcgaactcctggactcaagcaatcctcccacctcagcctcccaaagtgctgggattacaggcatgagccactgtgcccgacctaggaaatttgatttttaatatacattttattctagttgacttcctaatctcctatatgattgcctgcttcttctaacgtgtcatttttgttttttaggattaaaagaaggatcttattgttaataccaaagtggctttatgaagatattcctaatatgaaaaacagcaatgttgtgaaaatgctacaggtaacctaacatcatccaacaaaaactgagtggcaattgagaccaagagtgcagtctaataattagaatagaatttccattcaaataaataaatgtgcacacatattaacataattatatagacatgtgtaaagacaaaactgtacctgtttcaaatcatacttaaaagagatgagacaatagagttgggggaaagaaaacagagaagaaataaagaaacaaacctgttcaaaagagaaaagtgaagtggaagattgtgggccttgtgcacgagtcacctggtgaccaaacaatggtagatgtcttcaggaacaaagggagtagggaagaaaagctcaatgaaatgagcctcaaagattcaaagggcttctgtttcacatgagatggagagccaggtggagcccaagaagggcatcattttactctttaaacccagtggaatttgagaaaagcaatgtgagagaactgtgaacaatagtgtcacaggggtgggctaggtttccattctggcaaagagaaggccacacaccaggaagcccctgagggtacagggacattactgattataaaggagggaaggaacaagctatgtgtgttcctgataacccctggccctcgggattggctgtcaaggggctcaaaacccagtccaagggacaaacacatcatccaagccttgcaatgcagtgatgtaagtgcaatgatagaaatgagcccaaacatgtgatgggagtggaaaggaaacactgaaaggagaggagctgctcaggagaagcagggagaattcaaagtcagggggacccctgggggttagaactgcatgttgggtaagaagatgaggtgtttgtaaaacaaatagaagtggctctgtttcaagctctggtaagcctattagctaactctttccccaacctcatgtcatctgaacaaagggtttctaggctaaaaataaaatactttttaaaagttcaaaaacaactggtcaacagaatagagtctgagttctgtaacacaagacttctgtgatctgatccactcaccattccagctttactccagccactcctcacgtcaagatttttgatgaagcaatacccaatgtgctgttcttacccaaacctggcaagctccctaaggaggctggggctgaagaaaattggaatgacacttcatgtctgtttattcaagatctccttctttgctaaactcctttcctattctgggctcctggaattagaggcaaaccttagggcttccctcagataaggttttgttttctgcccgagagactaaggaatgcttagtcggcttgggtggcttaaacaacaaacatttatttgtcacagtgctggaggctgggaagtccatgatcaaggagccagtcaattcacttgctagtgagggctgtcttgtggacagctgccttctcacatggcagaggaagagatcatctctttcctgtctcttcttcactaatcccattcatgaaggctccgccctcatgacctaattaccacccaaagtctccactttcaaataacatcacattggcttcaatgtgaggggggctggggagagggcacacaaacgtttagtccgtaacagggggctataatattataattgtgttgggtgacagtttgcccaggaaggccagccatctgttggaaggcctccagtccatttattgcccagaaaagtcatcaggctaaaaccttcacccaccctccttatcatcacaaacatgtctgaattgtgagtagctttatttatttatttgttgttttttgttttgagacagagtcttgctctgtcacccaggctagagtgcagtggcatgatctcagctcactgcaacctccgcctcccgggctcaagcaattctcctgtctcagcctcctgagtagctgggattacaggcttgcgctactacgcctggctgatttttgtatttttagtagagacagggtttcaccatgttggccaggctggtcttgaactcctgatctcaagtgatctgcctgccttggcctcaaagtgctgggattacaggccttatccacctttagaacctcggcatcaagcatttgtcacacactcattctcggtatgtgagcaaaataggattctccccaacctttttttatttaagcaacaaagtattttcttgtaacaagaaatattaatattaatattcttgcctcagaagaccaacttttaaaaggccctaaaattaagtaataagtaataagttaatatataagactcaacttttctctttaaaacttttaaaccaagaaatgtgcttaagaagtggattatttttactattccttttcttcagtcttctcatgcaaatttaataaagatttggaagcaagcaggataacaaaagacaaaaggaggaaatcatatgggaagataaacaatatgagaaaatggagggagaaaggaaagttgaaagaaagcaaaattcctatcctcattcaattatccagttggttcttttaaatgtcttttgcaaaataaaatgatgtctttttttcctaggaaaatagtgaacttatgaataataattccagtgagcaggtcctatatgttgatcccatgattacagagataaaagaaatcttcatcccagaacacaagcctacagactacaagaaggagaatacaggacccctggagacaagagactacccgcaaaactcgctattcgacaatactacagttgtatatattcctgatctcaacactggatataaaccccaaatttcaaattttctgcctgagggaagccatctcagcaataataatgaaattacttccttaacacttaaaccaccagttgattccttagactcaggaaataatcccaggttacaaaagcatcctaattttgctttttctgtttcaagtgtgaattcactaagcaacacaatatttcttggagaattaagcctcatattaaatcaaggagaatgcagttctcctgacatacaaaactcagtagaggaggaaaccaccatgcttttggaaaatgattcacccagtgaaactattccagaacagaccctgcttcctgatgaatttgtctcctgtttggggatcgtgaatgaggagttgccatctattaatacttattttccacaaaatattttggaaagccacttcaataggatttcactcttggaaaagtagagctgtgtggtcaaaatcaatatgagaaagctgccttgcaatctgaacttgggttttccctgcaatagaaattgaattctgcctctttttgaaaaaaatgtattcacatacaaatcttcacatggacacatgttttcatttcccttggataaatacctaggtaggggattgctgggccatatgataagcatatgtttcagttctaccaatcttgtttccagagtagtgacatttctgtgctcctaccatcaccatgtaagaattcccgggagctccatgcctttttaattttagccattcttctgcctcatttcttaaaattagagaattaaggtcccgaaggtggaacatgcttcatggtcacacatacaggcacaaaaacagcattatgtggacgcctcatgtattttttatagagtcaactatttcctctttattttccctcattgaaagatgcaaaacagctctctattgtgtacagaaagggtaaataatgcaaaatacctggtagtaaaataaatgctgaaaattttcctttaaaatagaatcattaggccaggcgtggtggctcatgcttgtaatcccagcactttggtaggctgaggtaggtggatcacctgaggtcaggagttcgagtccagcctggccaatatgctgaaaccctgtctctactaaaattacaaaaattagccggccatggtggcaggtgcttgtaatcccagctacttgggaggctgaggcaggagaatcacttgaaccaggaaggcagaggttgcactgagctgagattgtgccactgcactccagcctgggcaacaagagcaaaactctgtctggaaaaaaaaaaaaaa

SEQ ID NO: 3 - DNA, 20 bp, Mus musculus
atagaacaacagctcggatt

SEQ ID NO: 4 - DNA, 20 bp, Mus musculus
acaacaactacacgtccatc

SEQ ID NO: 5 - DNA, 20 bp, Mus musculus
cccttaagcactgccgacca

SEQ ID NO: 6 - DNA, 20 bp, Mus musculus
tgtgtcatttatgaatactc

SEQ ID NO: 7 - DNA, 20 bp, Mus musculus
accatctgaagagcacataa

SEQ ID NO: 8 - DNA, 20 bp, Mus musculus
atctccactgactcactgca

SEQ ID NO: 9 - RNA, 20 nt, Mus musculus
auagaacaacagcucggauu

SEQ ID NO: 10 - RNA, 20 nt, Mus musculus
acaacaacuacacguccauc

SEQ ID NO: 11 - RNA, 20 nt, Mus musculus
cccuuaagcacugccgacca

SEQ ID NO: 12 - RNA, 20 nt, Mus musculus
ugugucauuuaugaauacuc

SEQ ID NO: 13 - RNA, 20 nt, Mus musculus
accaucugaagagcacauaa

SEQ ID NO: 14 - RNA, 20 nt, Mus musculus
aucuccacugacucacugca

SEQ ID NO: 15 - DNA, 20 bp, Homo sapiens
gtgcagtacatagaagcatg

SEQ ID NO: 16 - DNA, 20 bp, Homo sapiens
acaacaactacacgtccatc

SEQ ID NO: 17 - DNA, 20 bp, Homo sapiens
ctacatagacacaaaatacg

SEQ ID NO: 18 - DNA, 20 bp, Homo sapiens
accagctgaagagtatgtaa

SEQ ID NO: 19 - DNA, 20 bp, Homo sapiens
ccctttacatactcttcagc

SEQ ID NO: 20 - DNA, 20 bp, Homo sapiens
acttcatcaggaatatctgg

SEQ ID NO: 21 - RNA, 20 nt, Homo sapiens
gugcaguacauagaagcaug

SEQ ID NO: 22 - RNA, 20 nt, Homo sapiens
acaacaacuacacguccauc

SEQ ID NO: 23 - RNA, 20 nt, Homo sapiens
cuacauagacacaaaauacg

SEQ ID NO: 24 - RNA, 20 nt, Homo sapiens
accagcugaagaguauguaa

SEQ ID NO: 25 - RNA, 20 nt, Homo sapiens
cccuuuacauacucuucagc

SEQ ID NO: 26 - RNA, 20 nt, Homo sapiens
acuucaucaggaauaucugg

SEQ ID NO: 27 - DNA, 20 bp, Homo sapiens
tgtcaattctttctttgatt

SEQ ID NO: 28 - DNA, 20 bp, Homo sapiens
tttaacagatcattccgaac

SEQ ID NO: 29 - DNA, 20 bp, Homo sapiens
ttaacagatcattccgaact

SEQ ID NO: 30 - DNA, 20 bp, Homo sapiens
cagatcattccgaactgggt

SEQ ID NO: 31 - DNA, 20 bp, Homo sapiens
ctgcaaaaacctacccagtt

SEQ ID NO: 32 - RNA, 20 nt, Homo sapiens
ugucaauucuuucuuugauu

SEQ ID NO: 33 - RNA, 20 nt, Homo sapiens
uuuaacagaucauuccgaac

SEQ ID NO: 34 - RNA, 20 nt, Homo sapiens
uuaacagaucauuccgaacu

SEQ ID NO: 35 - RNA, 20 nt, Homo sapiens
cagaucauuccgaacugggu

SEQ ID NO: 36 - RNA, 20 nt, Homo sapiens
cugcaaaaaccuacccaguu

SEQ ID NO: 37 - protein, 178 aa, Homo sapiens
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 38 - DNA, 20 bp, Homo sapiens
gaaccaagacccagacatca

SEQ ID NO: 39 - DNA, 20 bp, Homo sapiens
caaggcgcatgtgaactccc

SEQ ID NO: 40 - DNA, 20 bp, Homo sapiens
aaggcgcatgtgaactccct

SEQ ID NO: 41 - DNA, 20 bp, Homo sapiens
aggcgcatgtgaactccctg

SEQ ID NO: 42 - DNA, 20 bp, Homo sapiens
ggcgcatgtgaactccctgg

SEQ ID NO: 43 - DNA, 20 bp, Homo sapiens
gggagaacctgaagaccctc

SEQ ID NO: 44 - DNA, 20 bp, Homo sapiens
gcctcagcctgagggtcttc

SEQ ID NO: 45 - DNA, 20 bp, Homo sapiens
gggtcttcaggttctccccc

SEQ ID NO: 46 - DNA, 20 bp, Homo sapiens
ggtcttcaggttctccccca

SEQ ID NO: 47 - RNA, 20 nt, Homo sapiens
gaaccaagacccagacauca

SEQ ID NO: 48 - RNA, 20 nt, Homo sapiens
caaggcgcaugugaacuccc

SEQ ID NO: 49 - RNA, 20 nt, Homo sapiens
aaggcgcaugugaacucccu

SEQ ID NO: 50 - RNA, 20 nt, Homo sapiens
aggcgcaugugaacucccug

SEQ ID NO: 51 - RNA, 20 nt, Homo sapiens
ggcgcaugugaacucccugg

SEQ ID NO: 52 - RNA, 20 nt, Homo sapiens
gggagaaccugaagacccuc

SEQ ID NO: 53 - RNA, 20 nt, Homo sapiens
gccucagccugagggucuuc

SEQ ID NO: 54 - RNA, 20 nt, Homo sapiens
gggucuucagguucuccccc

SEQ ID NO: 55 - RNA, 20 nt, Homo sapiens
ggucuucagguucuccccca